(19) United States
(12) Patent Application Publication
(10) Pub. No.: US 2026/0223846 A1
(43) Pub. Date: Aug. 6, 2026

(54) PEPTIDE IN COMBINATION WITH FUNGICIDE COMPOSITIONS FOR FUNGAL CONTROL AND RELATED METHODS

(71) Applicant: FLAGSHIP PIONEERING INNOVATIONS VII, LLC, CAMBRIDGE, MA (US)

(72) Inventors: Hope Hsiao-Wang DANISON, Somerville, MA (US); David Barry KOLESKY, Arlington, VA (US); Sindhu Manubhai KRISHNANKUTTY, Stow, MA (US); Antonio Diego MARTINEZ, Beverly, MA (US); Yajie NIU, Lexington, MA (US); Sowmya Ranganayaki RAMACHANDRAN, Bedford, MA (US); Michka Gabrielle SHARPE, Cambridge, MA (US); Joseph Kernan BEDREE, Somerville, MA (US)

(21) Appl. No.: 19/150,471

(22) PCT Filed: Jan. 23, 2024
(86) PCT No.: PCT/US2024/012652

§ 371 Date: Jul. 23, 2025

Related U.S. Application Data
(60) Provisional application No. 63/481,318, filed on Jan. 24, 2023.
(60) Provisional application No. 63/481,323, filed on Jan. 24, 2023.
(60) Provisional application No. 63/481,326, filed on Jan. 24, 2023.
(60) Provisional application No. 63/481,331, filed on Jan. 24, 2023.
(60) Provisional application No. 63/481,335, filed on Jan. 24, 2023.
(60) Provisional application No. 63/481,338, filed on Jan. 24, 2023.
(60) Provisional application No. 63/481,345, filed on Jan. 24, 2023.
(60) Provisional application No. 63/481,353, filed on Jan. 24, 2023.

(51) Int. Cl. A01N 37/46; A01P 3/00; A61K 31/4196; A61K 31/7048; A61K 38/16; A61P 31/10

(52) U.S. Cl. CPC A01N 37/46; A01P 3/00; A61K 31/4196; A61K 31/7048; A61K 38/16; A61P 31/10

(57) ABSTRACT

The disclosure provides antifungal compositions comprising conidial germination-inhibiting (CGI) factors, CGI factor precursors, CGI factor fragments, and CGI factor motifs and fungicides. In addition, the disclosure provides methods of controlling or inhibiting fungal growth and infection, as well as methods of treating a fungal disease using the compositions comprising CGI factors, CGI factor precursors, CGI factor fragments, and CGI factor motifs and fungicides.

### CROSS-REFERENCE TO RELATED APPLICATIONS

This application claims the benefit of U.S. Provisional Application No. 63/481,318, filed Jan. 24, 2023, U.S. Provisional Application No. 63/481,323, filed Jan. 24, 2023, U.S. Provisional Application No. 63/481,326, filed Jan. 24, 2023, U.S. Provisional Application No. 63/481,331, filed Jan. 24, 2023, U.S. Provisional Application No. 63/481,335, filed Jan. 24, 2023, U.S. Provisional Application No. 63/481,338, filed Jan. 24, 2023, U.S. Provisional Application No. 63/481,345, filed Jan. 24, 2023, and U.S. Provisional Application No. 63/481,353, filed Jan. 24, 2023, each of which is hereby incorporated by reference in its entirety.

### REFERENCE TO AN ELECTRONIC SEQUENCE LISTING

The contents of the electronic sequence listing (237212000240SEQLIST.xml; Size: 6,007,980 bytes; and Date of Creation: Jan. 19, 2024) are herein incorporated by reference in their entirety.

### FIELD OF THE DISCLOSURE

The present disclosure relates to antifungal or fungicidal agents, compositions and organisms comprising the antifungal or fungicidal agents, and methods of inhibiting or controlling fungi, such as fungal pathogens, using antifungal or fungicidal agents.

### BACKGROUND OF THE DISCLOSURE

The fungal kingdom encompasses a diverse group of organisms, some of which can act as pathogens for a variety of hosts. Pathogenic fungi can have detrimental effects on both human and animal health, either by direct infection, or by indirect effects from a secreted toxin. Food rot and crop loss due to uncontrolled fungal pathogens of plants or plant products can also lead to significant agricultural and economic losses. Thus, a need exists for agents capable of controlling or inhibiting growth of fungal pathogens, as well as for compositions comprising the agents that can be used to control or inhibit fungal growth and treat fungal infections of biological systems.

### BRIEF SUMMARY OF THE DISCLOSURE

In one aspect of the disclosure, provided herein are methods of decreasing growth or reproduction of a fungus, comprising providing a fungus with an antifungal composition that comprises an effective amount of at least one conidial germination-inhibiting (CGI) factor, CGI factor precursor, CGI factor fragment, or CGI factor motif, wherein the CGI factor, CGI factor precursor, CGI factor fragment, or CGI factor motif comprises an amino acid sequence that has at least 80% sequence identity with a sequence selected from the group consisting of SEQ ID NO: 961-1920, SEQ ID NO: 1921-2189, SEQ ID NO: 2194-2210, SEQ ID NO: 2215-2243, SEQ ID NO: 2457-3361, SEQ ID NO: 5707-5731, and SEQ ID NO: 5755-5911, and wherein the nucleotide sequence encoding the at least one CGI factor, CGI factor precursor, CGI factor fragment, or CGI factor motif does not occur in the genome of the fungus; an effective amount of at least one fungicide that interferes with fungal cell wall or fungal cell membranes, fungal signal transduction-inhibiting fungicide, fungal respiration-inhibiting fungicide, or fungal amino acid and/or protein synthesis-inhibiting fungicide; and optionally, an agriculturally or pharmaceutically acceptable carrier; whereby the growth or reproduction of the fungus is decreased, relative to a control fungus not provided with the antifungal composition. In embodiments, the CGI factor, CGI factor precursor, or CGI factor fragment includes only standard or canonical amino acids. In embodiments, the CGI factor, CGI factor precursor, or CGI factor fragment includes one or more non-standard or non-canonical amino acids (e.g., N-formylmethionine, selenocysteine, pyrrolysine, 4-hydroxyproline, 5-hydroxylysine, gamma-carboxyglutamic acid, hypusine, 3-aminotyrosine, homoallylglycine, homopropargylglycine, azidohomoalanine, azidonorleucine, azidophenylalanine, acetylphenylalanine, propargyllysine, propargyloxyphenylalanine), D-amino acids, beta amino acids, and synthetically or enzymatically modified amino acids, including phosphorylated or glycosylated amino acids or amino acids labelled with moieties such as fluorophores and other photoreactive groups, cross-linkers, affinity reagents, and the like.

In another aspect of the disclosure, provided herein are methods of preventing or reducing disease caused by a fungal pathogen of a plant, comprising contacting one or more cells of the plant with an antifungal composition that comprises an effective amount of at least one conidial germination-inhibiting (CGI) factor, CGI factor precursor, CGI factor fragment, or CGI factor motif, wherein the CGI factor comprises an amino acid sequence that has at least 80% sequence identity with a sequence selected from the group consisting of an effective amount of at least one conidial germination-inhibiting (CGI) factor, CGI factor precursor, CGI factor fragment, or CGI factor motif, wherein the CGI factor, CGI factor precursor, CGI factor fragment, or CGI factor motif comprises an amino acid sequence that has at least 80% sequence identity with a sequence selected from the group consisting of SEQ ID NO: 961-1920, SEQ ID NO: 1921-2189, SEQ ID NO: 2194-2210, SEQ ID NO: 2215-2243, SEQ ID NO: 2457-3361, SEQ ID NO: 5707-5731, and SEQ ID NO: 5755-5911; an effective amount of at least one fungicide that interferes with fungal cell wall or fungal cell membranes, fungal signal transduction-inhibiting fungicide, fungal respiration-inhibiting fungicide, or fungal amino acid and/or protein synthesis-inhibiting fungicide; and an agriculturally acceptable carrier; whereby disease caused by the fungal pathogen is prevented or decreased in the plant, relative to a control plant not contacted with the antifungal composition.

In another aspect of the disclosure, provided herein are methods of treating a subject with a fungal disease, comprising: administering to the subject an antifungal composition that comprises an effective amount of at least one conidial germination-inhibiting (CGI) factor, CGI factor precursor, CGI factor fragment, or CGI factor motif, wherein the CGI factor, CGI factor precursor, CGI factor fragment, or CGI factor motif comprises an amino acid sequence that has at least 80% sequence identity with a sequence selected from the group consisting of SEQ ID NO: 961-1920, SEQ ID NO: 1921-2189, SEQ ID NO: 2194-2210, SEQ ID NO: 2215-2243, SEQ ID NO: 2457-3361, SEQ ID NO: 5707-5731, and SEQ ID NO: 5755-5911; an effective amount of at least one fungicide that interferes with fungal cell wall or fungal cell membranes, fungal signal transduction-inhibiting fungicide, fungal respiration-inhibiting fungicide, or fungal amino acid and/or protein synthesis-inhibiting fungicide; and optionally, a pharmaceutically acceptable carrier; whereby the fungal disease is prevented or decreased in the subject, relative to a control subject not provided with the antifungal composition.

In another aspect of the disclosure, provided herein are methods of providing an organism with resistance to a fungal pathogen of the organism, comprising: contacting the organism with an antifungal composition that comprises an effective amount of at least one conidial germination-inhibiting (CGI) factor, CGI factor precursor, CGI factor fragment, or CGI factor motif, wherein the CGI factor, CGI factor precursor, CGI factor fragment, or CGI factor motif comprises an amino acid sequence that has at least 80% sequence identity with a sequence selected from the group consisting of SEQ ID NO: 961-1920, SEQ ID NO: 1921-2189, SEQ ID NO: 2194-2210, SEQ ID NO: 2215-2243, SEQ ID NO: 2457-3361, SEQ ID NO: 5707-5731, and SEQ ID NO: 5755-5911; an effective amount of at least one fungicide that interferes with fungal cell wall or fungal cell membranes, fungal signal transduction-inhibiting fungicide, fungal respiration-inhibiting fungicide, or fungal amino acid and/or protein synthesis-inhibiting fungicide; whereby the organism becomes resistant to the fungal pathogen.

In another aspect of the disclosure, provided herein is an antifungal or fungicidal composition comprising: an effective amount of at least one conidial germination-inhibiting (CGI) factor, CGI factor precursor, CGI factor fragment, or CGI factor motif, wherein the CGI factor, CGI factor precursor, CGI factor fragment, or CGI factor motif comprises an amino acid sequence that has at least 80% sequence identity with a sequence selected from the group consisting of SEQ ID NO: 961-1920, SEQ ID NO: 1921-2189, SEQ ID NO: 2194-2210, SEQ ID NO: 2215-2243, SEQ ID NO: 2457-3361, SEQ ID NO: 5707-5731, and SEQ ID NO: 5755-5911; an effective amount of at least one fungicide that interferes with fungal cell wall or fungal cell membranes, fungal signal transduction-inhibiting fungicide, fungal respiration-inhibiting fungicide, or fungal amino acid and/or protein synthesis-inhibiting fungicide; and an agriculturally or pharmaceutically acceptable carrier.

Other aspects of the disclosure are related to methods of preventing or treating fungal diseases in organisms such as plants and animals, such as non-human animals; antifungal compositions formulated for use in agriculture or as therapeutics; methods of preventing or treating fungal infection or growth on a surface, including non-living surfaces; and compositions, such as a substrate or matrix, having antifungal properties, e.g., resistance to fungal contamination or growth.

### BRIEF DESCRIPTION OF THE DRAWINGS

FIG. 1 depicts a graph of percent inhibition for the combination of drosomycin with alpha factor fusion (PEP340; SEQ ID NO: 2240 or SEQ ID NO: 5752), showing that the combination has a greater inhibitory effect than each of the peptides alone.

### DETAILED DESCRIPTION

Unless defined otherwise, all technical and scientific terms used have the same meaning as commonly understood by one of ordinary skill in the art to which this invention belongs. Conventional methods are used for the procedures described herein, such as those provided in the art, and demonstrated in the Examples and various general references. Unless otherwise stated, nucleic acid sequences described herein are given, when read from left to right, in the 5′ to 3′ direction. Nucleic acid sequences can be provided as DNA or as RNA, as specified; disclosure of one necessarily defines the other, as is known to one of ordinary skill in the art; for example, disclosure of a coding DNA sequence defines the encoded messenger RNA sequence. Furthermore, because of known codon degeneracy, different nucleic acid sequences can encode the same polypeptide sequence, and such modified nucleic acid sequences (e.g., for the purposes of codon optimization for a given species) are within the scope of the present disclosure.

The term “comprise” is intended to mean “include”. Where a term is provided in the singular, it also contemplates aspects of the invention described by the plural of that term. The term “and/or” where used herein is to be taken as specific disclosure of each of the multiple specified features or components with or without another. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The following description sets forth exemplary methods, parameters, and the like. It should be recognized, however, that such description is not intended as a limitation on the scope of the present disclosure but is instead provided as a description of exemplary embodiments.

### Antifungal Compositions for Fungal Control

An aspect of the disclosure provides an antifungal composition including a conidial germination-inhibiting (CGI) factor, a CGI factor precursor, a CGI factor fragment, or a CGI factor motif and a fungicide interfering with fungal cell walls or fungal cell membranes, a fungicide inhibiting fungal lipid synthesis or transport or membrane integrity or function, a fungicide that interacts with and disrupts fungal cell membranes, or a fungicide that inhibits beta-glucan synthesis in the fungal cell wall for fungal control, wherein fungal control includes inhibition or reduction of conidial germination, fungal growth, or fungal reproduction, or is fungicidal (able to kill the fungus).

In some embodiments, the antifungal composition is applied to a plant, a plant part, a harvested part of a plant, a seed, or an area to be planted to control any variety of fungal plant pathogens. Plants and plant cells are of any species of interest, including dicots and monocots. Plants of interest include row crop plants, fruit-producing plants and trees, vegetables, trees, and ornamental plants including ornamental flowers, shrubs, trees, groundcovers, and turf grasses. Examples of commercially important cultivated crops, trees, and plants include: alfalfa (Medicago sativa), almonds (Prunus dulcis), apples (Malus x domestica), apricots (Prunus armeniaca, P. brigantine, P. mandshurica, P. mume, P. sibirica), artichoke (Cynara cardunculus var. scolymus), asparagus (Asparagus officinalis), avocado (Persea americana), bananas (Musa spp.), barley (Hordeum vulgare), beans (Phaseolus spp.), blueberries and cranberries (Vaccinium spp.), Brazil nut (Bertholletia excelsa), cacao (Theobroma cacao), calamansi (Citrus x microcarpa), canola and rapeseed or oilseed rape, (Brassica napus), Polish canola (Brassica rapa), and related cruciferous vegetables including broccoli, kale, cabbage, and turnips (Brassica carinata, B. juncea, B. oleracea, B. napus, B. nigra, and B. rapa, and hybrids of these), carnation (Dianthus caryophyllus), carrots (Daucus carota sativus), cashew (Anacardium occidentale), cassava (Manihot esculentum), celery (Apium graveolens), cherry (Prunus avium), chestnut (Castanea spp.), chickpea or garbanzo (Cicer arietinum), chicory (Cichorium intybus), chile peppers and other capsicum peppers (Capsicum annuum, C. frutescens, C. chinense, C. pubescens, C. baccatum), chrysanthemums (Chrysanthemum spp.), citron (Citrus medica), coconut (Cocos nucifera), coffee (wild and domesticated Coffea spp. including Coffea arabica, Coffea canephora, and Coffea liberica), cotton (Gossypium hirsutum L.), cowpea (Vigna unguiculata and other Vigna spp.), fava beans (Vicia faba), cucumber (Cucumis sativus), currants and gooseberries (Ribes spp.), date (Phoenix dactylifera), duckweeds (family Lemnoideae), eggplant or aubergine (Solanum melongena), elderberries (Sambucus spp.), eucalyptus (Eucalyptus spp.), flax (Linum usitatissumum L.), geraniums (Pelargonium spp.), ginger (Zingiber officinale), ginseng (Panax spp.), grapefruit (Citrus x paradisi), grapes (Vitis spp.) including wine grapes (Vitis vinifera and hybrids thereof), guava (Psidium guajava), hazelnut (Corylus avellana, Corylus spp.), hemp and cannabis (Cannabis sativa and Cannabis spp.), hops (Humulus lupulus), horseradish (Armoracia rusticana), irises (Iris spp.), jackfruit (Artocarpus heterophyllus), kiwifruits (Actinidia spp.), kumquat (Citrus japonica), lemon (Citrus limon), lentil (Lens culinaris), lettuce (Lactuca sativa), limes (Citrus spp.), lychee (Litchi chinensis), macadamias (Macadamia spp.), maize or corn (Zea mays L.), mandarin (Citrus reticulata), mango (Mangifera indica), mangosteen (Garcinia mangostana), melon (Cucumis melo), millets (Setaria spp., Echinochloa spp., Eleusine spp., Panicum spp., Pennisetum spp.), oats (Avena sativa), oil palm (Ellis quineensis), okra (Abelmoschus esculentus), olive (Olea europaea), onion (Allium cepa) and other alliums (Allium spp.), orange (Citrus sinensis), papaya (Carica papaya), parsnip (Pastinaca sativa), passionfruit (Passiflora edulis), pecan (Carya illinoinensis), peaches and nectarines (Prunus persica), pear (Pyrus spp.), pea (Pisum sativum), peanut (Arachis hypogaea), peonies (Paeonia spp.), persimmons (Diospyros kaki, Diospyros spp.), petunias (Petunia spp.), pineapple (Ananas comosus), pistachio (Pistacia vera), plantains (Musa spp.), plum (Prunus domestica), poinsettia (Euphorbia pulcherrima), pomelo (Citrus maxima), poplar (Populus spp.), potato (Solanum tuberosum), pumpkins and squashes (Cucurbita pepo, C. maxima, C. moschata), quince (Cydonia oblonga), raspberries (Rubus idaeus, Rubus occidentalis, Rubus spp.), rhubarbs (Rheum spp.), rice (Oryza sativa L.), roses (Rosa spp.), rubber (Hevea brasiliensis), rye (Secale cereale), safflower (Carthamus tinctorius L), satsuma (Citrus unshiu), sesame seed (Sesame indium), sorghum (Sorghum bicolor), sour orange (Citrus x aurantium), soursop (Annona muricata), soybean (Glycine max L.), strawberries (Fragaria spp., Fragaria x ananassa), sugar beet (Beta vulgaris), sugarcanes (Saccharum spp.), sunflower (Helianthus annuus), sweet potato (Ipomoea batatas), tamarind (Tamarindus indica), tangerine (Citrus tangerina), tea (Camellia sinensis), tobacco (Nicotiana tabacum L.), tomatillo (Physalis philadelphica), tomato (Solanum lycopersicum or Lycopersicon esculentum), tulips (Tulipa spp.), walnuts (Juglans spp. L.), watermelon (Citrulus lanatus), wheat (Triticum aestivum), and yams (Discorea spp.). Wild relatives of domesticated plants are also of interest.

Exemplary diseases which can be treated, with causative pathogen shown in parenthesis, include Alternaria Leaf and Fruit Spot (Alternaria alternata), Anthracnose (Colletotrichum acutatum), Leaf Blight (Seimatosporium lichenicola), Leaf Rust (Tranzschelia discolor), Scab (Cladosporium carpophilum), Shot Hole (Wilsonomyces carpophilus), Brown Rot Blossom Blight (Monilinia laxa, M. fructicola), Black Sigatoka (Mycosphaerella fijiensis), Yellow Sigatoka (Mycosphaerella musicola), Alternaria Fruit Rot (Alternaria spp.), Anthracnose Fruit Rot (Colletotrichum gloeosporoides), Botryosphaeria Canker (Botryosphaeria spp.), Leaf Spot and Blotch (Mycosphaerella spp., Septoria spp.), Mummyberry (Monilinia vaccinii-corymbosi), Phomopsis Leaf Spot, Twig Blight and Stem Canker (Phomopsis vaccinii), Powdery Mildew (Sphaerotheca spp.), Septoria Blight (Septoria spp.), Spur Blight (Didymella spp., Phoma spp.), Anthracnose (Spaceloma necator, Elsinos veneta), Botryosphaeria Canker (Botryosphaeria dothidea), Colletotrichum Rot (Colletotrichum gloeosporioides), Leaf Spot and Blotch (Mycosphaerella spp., Septoria rubi, Sphaerulina rubi), Powdery Mildew (Sphaerotheca macularis, Microphaera spp., Oidium spp.), Rosette or Double Blossom of Blackberries (Cercosporella rubi), Spur Blight (Didymella applanata), Blackberry Rust (Phragmidium spp.), Anthracnose (Colletotrichum fragariae), Leather Rot (Phytophthora cactorum), Powdery Mildew (Sphaerotheca macularis), Botrytis grey mould on Foliage (Botrytis cinerea), Seedling Root Rot, Basal Stem Rot (Rhizoctonia solani), Cottonball (Monilinia oxycocci), Fruit Rots (Physalospora vaccinia, Glomerella cingulata, Coleophoma empetri), Lophodermium Twig Blight (Lophodermium spp.), Fairy Ring Suppression (Psilocybe spp.), Albinism (Alternaria alternata pv citri), Alternaria Leaf and Fruit Spot (Alternaria citri), Anthracnose (Colletotrichum acutatum, C. gloeosporioides), Cercospora Leaf Spot (Cercospora spp.), Diplodia Stem-End Rot (Diplodia natalensis), Greasy Spot (Mycosphaerella citri), Melanose (Diaporthe citri), Penicillium Decays, Green Mold, Whisker Mold, Blue Mold (Penicillium spp.), Phomopsis Stem-End Rot (Phomopsis citrii), Post Bloom Fruit Drop (PFD) (Colletotrichum acutatum), Powdery Mildew (Erysiphe spp.), Scab (Elsinoe fawcettii), Sweet Orange Scab (Elsinoe australis), Black Spot (Guignardia citricarpa), Black Rot (Guignardia bidwellii), Downy Mildew (Plasmopara viticola), Phomopsis Cane and Leaf Spot (Phomopsis viticola), Powdery Mildew (Uncinula necator), Botrytis Bunch Rot (Botrytis cinerea), Aspergillus Crown Rot (Aspergillus niger), Pythium Damping Off (Pythium spp.), Stem Rot/White Mold (Sclerotium rolfsii), Rhizoctonia Peg and Pod Rot (Rhizoctonia solani), Stem Rot/White Mold (Sclerotium rolfsii), Cylindrocladium Black Rot (Cylindocladium crotalariae), Pythium Pod Rot (Pythium myriotylum), Alternaria Late Blight (Alternaria alternata), Botryosphaeria Panicle and Shoot Blight (Botryosphaeria dothidea), Septoria Leaf Spot (Septoria pistaciarum), Scab (Cladosporium carpophilum), Alternaria Spot and Fruit Rot (Alternaria alternata), Anthracnose (Colletotrichum prunicola, C. gloeosporioides), Leaf Rust (Tranzschelia discolor), Powdery Mildew (Sphaerotheca pannosa, Podosphaera clandestina), Shot Hole (Wilsonomyces carpophilus), Alternaria Leaf Spot (Alternaria spp., A. alternata), Ascochyta Leaf Spot (Ascochyta cynarae), Phyllostica Leaf Spot (Phyllostica spp.), Rust (Uromyces betae, Puccinia helianthi), White Rust (Albugo tragopogonis), Anthracnose (Colletotrichum acutatum, Glomerella cingulata), Eastern Filbert Blight (Anisogramma anomale), Late Blight (Alternaria alternata), Scab (Cladosporium carpophilum), Septoria Leaf Spot (Septoria pistaciarum), Shot Hole (Wilsonomyces carpophilus), Blossom Blight (Monilinia laxa, M. fructicola), Powdery Mildew (Erysiphe spp.), Rust (Puccinia spp.), Alternaria black spot (Alternaria brassicae), Black leg/Phoma (Leptosphaeria maculans), Cercospora leaf spot (C. brassicicola), Head rot (Rhizoctonia solani), Leaf spot and pod rot (Alternaria alternata), Powdery mildew (Erysiphe polygoni), Southern blight (Sclerotium rolfsii), Anthracnose leaf blight (Colletotrichum graminicola), Gray leaf spot (Cercospora sorghi), Northern corn leaf blight (Setosphaeria turcica), Northern corn leaf spot (Cochliobolus carbonum), Common Rust (Puccinia sorghi), Southern Rust (P. polysora), Southern corn leaf blight (Cochliobolus heterostrophus), Eye spot (Aureobasidium zeae), Physoderma brown spot (P. maydis), Yellow Leaf Blight (Phyllosticta maydis), Ascochyta blight (A. gossypii), Rust (Puccinia schedonnardi, P. cacabata), Rhizoctonia leaf and stem diseases (R. solani), Target spot (Corynespora cassiicola), Southern blight (Sclerotium rolfsii), Rhizoctonia limb rot (R. solani), Cylindrocladium black rot (C. crotalaria), White mold (Sclerotinia minor), Early leaf spot (Cercospora arachidicola), Late leaf spot (Cercosporidium personatum), Web blotch (Phoma arachidicola), Rust (Puccinia arachidis), Pepper Spot (Leptospherulina crassiasca), Southern stem rot (Sclerotium rolfsii), Rhizoctonia limb rot (R. solani), Cylindrocladium black rot (C. crotalaria), White mold (Sclerotinia minor), Anthracnose (Colletotrichum lindemuthianum), Ascochyta blight (A. phaseolorum), Cercospora leaf blotch (C. cruenta), Downy mildew (Phytophthora nicotianae), Rust (Uromyces appendiculatus), Anthracnose (ripe rot) (C. gloeosporoides), Mummy berry (M. vacciniicorymbosi), Rust (Pucciniastrum vaccinii), Septoria leaf spot (Septoria albopunctata), Downy mildew (Peronospora parasitica), Alternaria leaf blight (A. dauci), Cercospora leaf spot (C. carotae), Basal stalk rot (Rhizoctonia solani), Early blight (Cercospora apii), Late blight (Septoria apicola), Verticillium brown spot and dry bubble, Pink rot (Sclerotinia sclerotiorum), Lophodermium leaf/twig blight (L. hypophyllum), Upright dieback (Phomopsis vaccinii), Anthracnose (Colletotrichum spp.), Downy mildew (Pseudoperonospora cubensis), Target spot (Corynespora cassiicola), Alternaria leaf blight (A. cucumerina), Alternaria leaf spot (A. alternata), Cercospora leaf spot (C. citrullina), Gummy stem blight/vine decline (Didymella bryoniae), Powdery mildew (Sphaerotheca only), Scab (Cladosporium cucumerinum), Anthracnose (Colletotrichum spp.), Botrytis leaf mold (Botrytis cinerea), Cercospora leaf spot (Cercospora spp.), Powdery mildew (Leveillula taurica), Purple blotch (Alternaria porri), Botrytis neck rot, Downy mildew (Peronospora destructor), Early leaf spot (Cercospora arachidicola), Late leaf spot (Cercosporidium personatum), Pepper spot (Leptosphaerulina crassiasca), Black dot (Colletotrichum coccodes), Botrytis vine rot (B. cinerea), Early blight (Alternaria solani), Late blight (Phytophthora infestans), Anthracnose (Colletotrichum truncatum), Cercospora leaf blight (C. kikuchii), Diaporthe pod and stem rot (D. phaseolorum), Frogeye leaf spot (Cercospora sojina), Purple seed stain (C. kikuchii), Septoria brown spot (S. glycines), Rust (Phakopsora pachyrhizi), Stem canker (Diaporthe phaseolorum), Early blight (Alternaria solani), Gray leaf mold (Fluvia fluva; Cladosporium), Gray leaf spot (Stemphyllium botryosum), Late blight (Phytophthora infestans), Septoria leaf spot (S. lycopersici), Target spot (Corynespora cassiicola), Alternaria fruit rot (black mold) (A. alternata), Anthracnose (Colletotrichum spp.), Botrytis gray mold (B. cinerea), Late blight fruit rot (P. infestans), Rhizoctonia fruit rot (R. solani), Anthracnose (Colletotrichum gloeosporioides), Anthracnose (Colletotrichum acutatum), Blossom blight/brown rot (Monilinia spp.), Scab (Venturia carpophila), Shot hole (Wilsonomyces carpophilus), Leaf curl (Taphrina deformans), Black knot (cherry, plum) (Apiosporina morbosa), Cherry leaf spot (Blumeriella jaapii), Scab (Cladosporium carpophilum), Interior needle blight (Mycosphaerella spp. and Phaeocryptopus nudus), Swiss needlecast (Phaeocryptopus gaeumannii), Interior needle blight (Mycosphaerella spp. and Phaeocryptopus nudus), Scleroderris canker (Gremmeniella abietina), Leaf rust (Thekopsora minima), Powdery mildew (Erysiphe necator), Alternaria rot (A. alternata), Angular leaf spot (Mycosphearella angulata), Anthracnose (Elsinoe ampelina), Black Rot (Guignardia bidwellii), Leaf Blight (Pseudocercospora vitis), Phomopsis cane and leaf spot (P. viticola), Rotbrenner (Pseudopezicula tracheiphila), Septoria leaf spot (S. ampelina), Apple Scab (Venturia inaequalis), Pear Scab (V. piris), Alternaria blotch, Alternaria rot (Alternaria spp.), Cedar apple rust (Gymnosporangium juniper-virginianae), Powdery mildew (Podosphaera leucotricha), Quince rust (Gymnosporangium spp.), Flyspeck and Sooty blotch, Bitter rot (Glomerella cingulata), Black rot (Botryosphaeria obtusa), Brooks fruit spot (Mycosphaerella pomi), White rot (Botryosphaeria dothidea), Alternaria rot and surface mold, Bitter rot Blue mold, Bull's-eye rot, Gray mold, Phacidiopycnis rot, Rhizopus rot, Speck rot, Sphaeropsis rot, White rot, Damping off (Pythium spp.), Root Rot (Phytophthora spp.), Leather rot (P. cactorum), Red stele (P. fragariae), Vascular collapse (P. cactorum), Basal stem rot (Phytophthora spp.), Crown rot (Phytophthora capsici), Downy Mildew (Peronospora effusa; P. farinosa), White rust (Albugo occidentalis), Pink rot (Phytophthora erythroseptica), Pythium leak, Pythium seedling disease (Pythium spp.), Phytophthora root and stem rot (Phytophthora megasperma), Pythium damping off (Pythium spp.), Collar rot, Crown rot, Root rot (Phytophthora spp.), Crown rot, Spear rot (Phytophthora spp.), Root Rot (Phytophthora cinnamomi), Downy mildew (Peronospora parasitica), Brown rot, Citrus foot rot, Gummosis, Root rot, Trunk canker (Phytophthora spp.), or Downy mildew (Bremia lactucae). From an agricultural or horticultural perspective, and for the purposes of this application, some of the pathogens and diseases listed above are considered “fungal” although the causative pathogen is technically an oomycete (phylum Oomycota), including, but not limited to Pythion spp., Phytophthora spp., Peronospora spp., Plasmopara spp., Albugo spp., and Bremia spp.

When applying to a harvested part of a plant (also referred to herein as post-harvest), application can be by a variety of treatment methods, e.g., dip, drip, drench, spray, or fog. In certain embodiments, application is made to a plant prior to harvest, e.g., by spraying on or injecting into a plant, prior to harvesting the plant or a part of the plant (such as a fruit, seed, or leaf). In certain embodiments, the application is by injection into the vascular system of the plant. In alternative embodiments, the harvested plant part has applied to it a composition, such as a film or membrane, containing the CGI factor, CGI factor precursor, CGI factor fragment, or CGI factor motif and/or the fungicide, or is packaged in a container that includes the CGI factor, CGI factor precursor, CGI factor fragment, or CGI factor motif and/or the fungicide. Such treatments, compositions, and containers are further useful for protecting foodstuffs (e.g., processed food products such as bakery goods or processed fruit or vegetables) from fungal growth and spoilage. Any of the plants or plant parts described above can be treated post-harvest. In some embodiments, plants treated post-harvest will include alfalfa, almonds, apples, apricots, artichoke, asparagus, avocado, bananas, barley, beans, blueberries and cranberries, Brazil nut, cacao, calamansi, canola and rapeseed or oilseed rape, Polish canola, and related cruciferous vegetables including broccoli, kale, cabbage, and turnips, carnation, carrots, cashew, cassava, celery, cherry, chestnut, chickpea or garbanzo, chicory, chili peppers and other Capsicum peppers, chrysanthemums, citron, coconut, coffee, cotton, cowpea, fava beans, cucumber, currants and gooseberries, date, duckweeds, eggplant or aubergine, elderberries, Eucalyptus, flax, geraniums, ginger, Ginseng, grapefruit, grapes including wine grapes, guava, hazelnut, hemp and Cannabis, hops, horseradish, irises, jackfruit, kiwifruits, kumquat, lemon, lentil, lettuce, limes, lychee, macadamias, maize or corn, mandarin, mango, mangosteen, melon, millets, oats, oil palm, okra, olive, onion and other alliums, orange, Papaya, parsnip, passionfruit, pecan, peaches and nectarines, pear, pea, peanut, peonies, persimmons, petunias, pineapple, pistachio, plantains, plum, poinsettia, pomelo, poplar, potato, pumpkins and squashes, quince, raspberries, rhubarbs, rice, roses, rubber, rye, safflower, satsuma, sesame seed, sorghum, sour orange, soursop, soybean, strawberries, sugar beet, sugarcanes, sunflower, sweet potato, tamarind, tangerine, tea, tobacco, tomatillo, tomato, tulips, walnuts, watermelon, wheat, and yams.

In some embodiments, the antifungal composition is applied to an animal or a microbe. In some embodiments, the animal is a domestic animal, a livestock animal, a mammal, or a non-mammal. In some embodiments, the non-mammal is a reptile, an insect, an amphibian, a bird, or a fish. In embodiments, the subject is a vertebrate animal (e.g., mammal, bird, cartilaginous or bony fish, reptile, or amphibian). In embodiments, the subject is a human; including adults and non-adults (infants and children). In embodiments, the subject is a non-human mammal, such as a non-human primate (e.g., monkeys, apes), ungulate (e.g., cattle, buffalo, bison, sheep, goat, pig, camel, llama, alpaca, deer, horses, donkeys), carnivore (e.g., dog, cat), rodent (e.g., rat, mouse), or lagomorph (e.g., rabbit). In embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots). In embodiments, the subject is an invertebrate such as an arthropod (e.g., insects, arachnids, crustaceans), a nematode, an annelid, a helminth, or a mollusc. In embodiments, the subject is an organism that is part of a symbiosis, such as part of the microbiome of an animal or a plant. In certain optional embodiments, the methods disclosed herein are not methods for treatment of the human or animal body by surgery or therapy. In certain optional embodiments, the subject to which the antifungal composition is applied or provided is not a human, human tissue, human embryo, or human cell in any stage of formation and development.

In some embodiments, the CGI factor, CGI factor precursor, CGI factor fragment, or CGI factor motif and/or the fungicide is active and/or toxic to a structural element fungal pathogen (e.g., a fungal pathogen that infests or damages human-built structures such as buildings or other human-created artifacts, or components thereof). In some embodiments, the CGI factor, CGI factor precursor, CGI factor fragment, or CGI factor motif and/or the fungicide inhibits growth or reproduction of or is toxic to a fungus that damages wood or other materials useful in human-built structures or artifacts; examples include wood-decaying fungi that cause brown rot, white rot, or soft rot, or fungi that cause dry rot in human-built structures or buildings. In some embodiments, the structural element fungal pathogen is dry rot fungus (Serpula lacrymans), cellar rot fungus (Coniphora puteana), a wet rot fungus (Antrodia vaillantii, A. xantha, Asterostroma spp., Donkioporia expansa, Paxillus panuoides, Phellinus contignuus, Tyromyces placentus), or a fungus that colonizes water-damaged structural materials, e.g., Penicillium chrysogenum, Aspergillus versicolor, Chaetomium spp., Acremonium spp., Ulocladium spp., Stachybotrys spp., Arthrinium phaeospermum, Aureobasidium pullulans, Cladosporium herbarum, Trichoderma spp., Aspergillus fumigatus, Aspergillus melleus, Aspergillus niger, Aspergillus ochraceus, Mucor racemosus, or Mucor spinosus.

### CGI Factors

In various embodiments, a CGI factor of the present disclosure is an alpha pheromone, a homodimer of an alpha pheromone (i.e., of an alpha factor monomer), a homodimer of an alpha pheromone with a linker, or an alpha pheromone fused to a cell-penetrating peptide (CPP). Embodiments of alpha pheromones are peptides natively expressed by a fungus, e.g., a fungal pathogen. In embodiments, an alpha pheromone encoded in the genome of one fungal genus, or a derivative of the alpha pheromone, is effective in reducing viability of cells of a different fungal genus. A CGI factor effectively inhibits growth of a fungal pathogen on or within an organism, wherein the amino acid sequence of the CGI factor is not that of an alpha pheromone natively expressed by the fungal pathogen, or wherein the nucleotide sequence encoding the CGI factor does not occur in the genome of the fungal pathogen. Examples of an alpha pheromone include SEQ ID NO: 2182, SEQ ID NO: 2183, SEQ ID NO: 2184, SEQ ID NO: 2185, SEQ ID NO: 2186, and SEQ ID NO: 2187. Homodimers of these alpha pheromone monomers, e.g., homodimers of a single fungal alpha pheromone peptide, either with or without a linker, are also disclosed.

Homodimers or alpha pheromones fused to a CPP are synthetic peptides. Homodimers with linkers have linking amino acids adjacent to or in between the alpha pheromone monomers. These peptides are longer than the naturally occurring alpha pheromone monomers, and in embodiments, antifungal activity is improved by selecting a longer CGI peptide or by increasing the length of a CGI peptide sequence. In embodiments, linkers include one or more neutral amino acids, e.g., one or more glycine residues (such as GGGG (SEQ ID NO: 5834)), as well as SEQ ID NO: 5701, SEQ ID NO: 5702, SEQ ID NO: 5703, SEQ ID NO: 5704, SEQ ID NO: 5705, or SEQ ID NO: 5706. In some embodiments, homodimers include SEQ ID NO: 2188, SEQ ID NO: 5835, SEQ ID NO: 5836, SEQ ID NO: 5837, SEQ ID NO: 5838, SEQ ID NO: 5839, or SEQ ID NO: 5840. In some embodiments, homodimers with a GGGG (SEQ ID NO: 5834) linker include SEQ ID NO: 5837, SEQ ID NO: 5841, SEQ ID NO: 5842, SEQ ID NO: 5843, SEQ ID NO: 5844, or SEQ ID NO: 5845. In some embodiments, homodimers with linkers include SEQ ID NO: 5846, SEQ ID NO: 5847, SEQ ID NO: 5848, SEQ ID NO: 5849, SEQ ID NO: 5850, SEQ ID NO: 5851, SEQ ID NO: 5852, SEQ ID NO: 5853, SEQ ID NO: 5854, SEQ ID NO: 5855, SEQ ID NO: 5856, SEQ ID NO: 5857, SEQ ID NO: 5858, SEQ ID NO: 5859, SEQ ID NO: 5860, SEQ ID NO: 5861, SEQ ID NO: 5862, SEQ ID NO: 5863, SEQ ID NO: 5864, SEQ ID NO: 5865, SEQ ID NO: 5866, SEQ ID NO: 5867, SEQ ID NO: 5868, SEQ ID NO: 5869, SEQ ID NO: 5870, SEQ ID NO: 5871, SEQ ID NO: 5872, SEQ ID NO: 5873, SEQ ID NO: 5874, SEQ ID NO: 5875, SEQ ID NO: 5876, SEQ ID NO: 5877, SEQ ID NO: 5878, SEQ ID NO: 5879, SEQ ID NO: 5880, SEQ ID NO: 5881, SEQ ID NO: 5882, SEQ ID NO: 5883, SEQ ID NO: 5884, SEQ ID NO: 5885, SEQ ID NO: 5886, SEQ ID NO: 5887, SEQ ID NO: 5888, SEQ ID NO: 5889, SEQ ID NO: 5890, SEQ ID NO: 5891, SEQ ID NO: 5892, SEQ ID NO: 5893, SEQ ID NO: 5894, SEQ ID NO: 5895, SEQ ID NO: 5896, SEQ ID NO: 5897, SEQ ID NO: 5898, SEQ ID NO: 5899, SEQ ID NO: 5900, SEQ ID NO: 5901, SEQ ID NO: 5902, SEQ ID NO: 5903, SEQ ID NO: 5904, SEQ ID NO: 5905, SEQ ID NO: 5906, SEQ ID NO: 5907, SEQ ID NO: 5908, SEQ ID NO: 5909, SEQ ID NO: 5910, or SEQ ID NO: 5911.

Another embodiment of these synthetic peptides is a synthetic peptide that includes sequence of, or derived from, at least one CGI peptide, fused to a cell-penetrating peptide (CPP). Hundreds of CPP sequences have been described; see, e.g., the database of cell-penetrating peptides, CPPsite, publicly available at crdd[dot]osdd[dot]net/raghava/cppsite/. An example of a commonly used CPP sequence is a poly-arginine sequence, e.g., octaarginine or nonaarginine, which can be fused to the C-terminus of the CGI peptide. Other embodiments of synthetic antifungal peptides include peptides that have the amino acid sequence of at least one CGI peptide of the present disclosure fused at its C-terminus to a polyarginine CPP sequence (e.g., Octaarginine, SEQ ID NO: 5747; Nonaarginine, SEQ ID NO: 5748), a polylysine CPP sequence (e.g., Octalysine, SEQ ID NO: 5749), a Tat basic domain sequence (RKKRRQRRR, SEQ ID NO: 5736), a BP100 CPP sequence (KKLFKKILKYL, SEQ ID NO:5737), a D-R9 CPP sequence (rrrrrrrrr (D form), SEQ ID NO:5738), a KLA10 CPP sequence (KALKKLLAKWLAAAKALL, SEQ ID NO:5739), a dhvar5 CPP sequence (LLLFLLKKRKKRKY, SEQ ID NO:5740), an HPV33L2-445/467 CPP sequence (SYFILRRRRKRFPYFFTDVRVAA, SEQ ID NO:5741), a Crot(27-29) derivative(2) CPP sequence (KMDCRWRWKCCKK, SEQ ID NO:5742), a CyLoP-1 CPP sequence (CRWRWKCCKK, SEQ ID NO:5743), an M511 CPP sequence (FLGKKFKKYFLQLLK, SEQ ID NO:5744), a E162 CPP sequence (KTVLLRKLLKLLVRKI, SEQ ID NO:5745), an MG2d CPP sequence (GIGKFLHSAKKWGKAFVGQIMNC, SEQ ID NO:5746), or another CPP sequence (e.g., cell-penetrating peptide sequences publicly disclosed at CPPsite, crdd[dot]osdd[dot]net/raghava/cppsite/or disclosed in Numata et al. (2018) Scientific Reports, 8:10966, DOI:10.1038/s41598-018-29298-6). Examples of fusion peptide sequences (SEQ ID NO: 5755-5828) are provided in Table 1, below, with the alpha pheromone sequences provided at left, and the CPP sequences provided at top.

TABLE 1
Fusion peptide sequences

| Octaarginine | Nonaarginine | Octalysine | SEQ | SEQ | SEQ | SEQ | SEQ | SEQ | SEQ | SEQ | SEQ | SEQ | SEQ |
|---|---|---|---|---|---|---|---|---|---|---|---|---|---|
| (SEQ | (SEQ | (SEQ | ID | ID | ID | ID | ID | ID | ID | ID | ID | ID | ID |
| ID | ID | ID | NO: | NO: | NO: | NO: | NO: | NO: | NO: | NO: | NO: | NO: | NO: |
| NO: | NO: | NO: | 5736 | 5737 | 5738 | 5739 | 5740 | 5741 | 5742 | 5743 | 5744 | 5745 | 5746 |
| 5747) | 5748) | 5749) | | | | | | | | | | | |

| SEQ | SEQ | SEQ |
|---|---|---|
| ID NO.: 2184 | ID NO.: 2183 | ID NO.: 2182 |
| WHWLQLKPGQPMYRRRRRRRR (SEQ ID | WCGRPGQPCRRRRRRRR (SEQ ID NO: 5751) | WCTWKGQPCWRRRRRRRR (SEQ ID |
| NO: 5752) | | NO: 5750) |
| WHWLQLKPGQPMYRRRRRRRRR (SEQ ID | WCGRPGQPCRRRRRRRRR (SEQ ID | WCTWKGQPCWRRRRRRRRR (SEQ ID |
| NO: 5758) | NO: 5757) | NO: 5756) |
| WHWLQLKPGQPMYKKKKKKKK (SEQ ID | WCGRPGQPCKKKKKKKK (SEQ ID NO: 5763) | WCTWKGQPCWKKKKKKKK (SEQ ID |
| NO: 5764) | | NO: 5762) |
| WHWLQLKPGQPMYRKKRRQRRR (SEQ ID | WCGRPGQPCRKKRRQRRR (SEQ ID | WCTWKGQPCWRKKRRQRRR (SEQ ID |
| NO: 5770) | NO: 5769) | NO: 5768) |
| WHWLQLKPGQPMYKKLFKKILKYL (SEQ ID | WCGRPGQPCKKLFKKILKYL (SEQ ID | WCTWKGQPCWKKLFKKILKYL (SEQ ID |
| NO: 5776) | NO: 5775) | NO: 5774) |
| WHWLQLKPGQPMYRRRRRRRRR (SEQ ID | WCGRPGQPCRRRRRRRRR (SEQ ID | WCTWKGQPCWRRRRRRRRR (SEQ ID |
| NO: 5782) | NO: 5781) | NO: 5780) |
| WHWLQLKPGQPMYKALKKLLAKWLAAAKA | WCGRPGQPCKALKKLLAKWLAAAKALL (SEQ | WCTWKGQPCWKALKKLLAKWLAAAKALL |
| LL (SEQ ID NO: 5788) | ID NO: 5787) | (SEQ ID NO: 5786) |
| WHWLQLKPGQPMYLLLFLLKKRKKRKY | WCGRPGQPCLLLFLLKKRKKRKY (SEQ ID | WCTWKGQPCWLLLFLLKKRKKRKY (SEQ |
| (SEQ ID NO: 5794) | NO: 5793) | ID NO: 5792) |
| WHWLQLKPGQPMYSYFILRRRRKRFPYFF | WCGRPGQPCSYFILRRRRKRFPYFFTDVRVA | WCTWKGQPCWSYFILRRRRKRFPYFFTD |
| TDVRVAA (SEQ ID NO: 5800) | A (SEQ ID NO: 5799) | VRVAA (SEQ ID NO: 5798) |
| WHWLQLKPGQPMYKMDCRWRWKCCKK | WCGRPGQPCKMDCRWRWKCCKK (SEQ ID | WCTWKGQPCWKMDCRWRWKCCKK |
| (SEQ ID NO: 5806) | NO: 5805) | (SEQ ID NO: 5804) |
| WHWLQLKPGQPMYCRWRWKCCKK (SEQ | WCGRPGQPCCRWRWKCCKK (SEQ ID | WCTWKGQPCWCRWRWKCCKK (SEQ ID |
| ID NO: 5812) | NO: 5811) | NO: 5810) |
| WHWLQLKPGQPMYFLGKKFKKYFLQLLK | WCGRPGQPCFLGKKFKKYFLQLLK (SEQ ID | WCTWKGQPCWFLGKKFKKYFLQLLK |
| (SEQ ID NO: 5818) | NO: 5817) | (SEQ ID NO: 5816) |
| WHWLQLKPGQPMYKTVLLRKLLKLLVRKI | WCGRPGQPCKTVLLRKLLKLLVRKI (SEQ ID | WCTWKGQPCWKTVLLRKLLKLLVRKI |
| (SEQ ID NO: 5824) | NO: 5823) | (SEQ ID NO: 5822) |
| WHWLQLKPGQPMYGIGKFLHSAKKWGKA | WCGRPGQPCGIGKFLHSAKKWGKAFVGQIM | WCTWKGQPCWGIGKFLHSAKKWGKAFV |
| FVGQIMNC (SEQ ID NO: 5830) | NC (SEQ ID NO: 5829) | GQIMNC (SEQ ID NO: 5828) |
| SEQ | SEQ | SEQ |
| ID NO.: 2187 | ID NO.: 2186 | ID NO.: 2185 |
| GWHWLQLKPGQPMYRRRRRRRR (SEQ ID | WHWLQLKPGQPMYGRRRRRRRR (SEQ ID NO: 5754) | WKMGQYHQLPPLWRRRRRRRR (SEQ ID |
| NO: 5755) | | NO: 5753) |
| GWHWLQLKPGQPMYRRRRRRRRR (SEQ ID | WHWLQLKPGQPMYGRRRRRRRRR (SEQ ID NO: 5760) | WKMGQYHQLPPLWRRRRRRRRR (SEQ ID |
| NO: 5761) | | NO: 5759) |
| GWHWLQLKPGQPMYKKKKKKKK (SEQ ID | WHWLQLKPGQPMYGKKKKKKKK (SEQ ID NO: 5766) | WKMGQYHQLPPLWKKKKKKKK (SEQ ID |
| NO: 5767) | | NO: 5765) |
| GWHWLQLKPGQPMYRKKRRQRRR (SEQ ID | WHWLQLKPGQPMYGRKKRRQRRR (SEQ ID NO: 5772) | WKMGQYHQLPPLWRKKRRQRRR (SEQ ID |
| NO: 5773) | | NO: 5771) |
| GWHWLQLKPGQPMYKKLFKKILKYL (SEQ ID | WHWLQLKPGQPMYGKKLFKKILKYL (SEQ ID NO: 5778) | WKMGQYHQLPPLWKKLFKKILKYL (SEQ ID |
| NO: 5779) | | NO: 5777) |
| GWHWLQLKPGQPMYRRRRRRRRR (SEQ ID | WHWLQLKPGQPMYGRRRRRRRRR (SEQ ID NO: 5784) | WKMGQYHQLPPLWRRRRRRRRR (SEQ ID |
| NO: 5785) | | NO: 5783) |
| GWHWLQLKPGQPMYKALKKLLAKWLAAAKALL | WHWLQLKPGQPMYGKALKKLLAKWLAAAKALL (SEQ | WKMGQYHQLPPLWKALKKLLAKWLAAAKA |
| (SEQ ID NO: 5791) | ID NO: 5790) | LL (SEQ ID NO: 5789) |
| GWHWLQLKPGQPMYLLLFLLKKRKKRKY (SEQ ID | WHWLQLKPGQPMYGLLLFLLKKRKKRKY (SEQ ID | WKMGQYHQLPPLWLLLFLLKKRKKRKY |
| NO: 5797) | NO: 5796) | (SEQ ID NO: 5795) |
| GWHWLQLKPGQPMYSYFILRRRRKRFPYFFTDVR | WHWLQLKPGQPMYGSYFILRRRRKRFPYFFTDVRVAA | WKMGQYHQLPPLWSYFILRRRRKRFPYFFT |
| VAA (SEQ ID NO: 5803) | (SEQ ID NO: 5802) | DVRVAA (SEQ ID NO: 5801) |
| GWHWLQLKPGQPMYKMDCRWRWKCCKK (SEQ | WHWLQLKPGQPMYGKMDCRWRWKCCKK (SEQ ID | WKMGQYHQLPPLWKMDCRWRWKCCKK |
| ID NO: 5809) | NO: 5808) | (SEQ ID NO: 5807) |
| GWHWLQLKPGQPMYCRWRWKCCKK (SEQ ID | WHWLQLKPGQPMYGCRWRWKCCKK (SEQ ID | WKMGQYHQLPPLWCRWRWKCCKK (SEQ |
| NO: 5815) | NO: 5814) | ID NO: 5813) |
| GWHWLQLKPGQPMYFLGKKFKKYFLQLLK (SEQ | WHWLQLKPGQPMYGFLGKKFKKYFLQLLK (SEQ ID | WKMGQYHQLPPLWFLGKKFKKYFLQLLK |
| ID NO: 5821) | NO: 5820) | (SEQ ID NO: 5819) |
| GWHWLQLKPGQPMYKTVLLRKLLKLLVRKI (SEQ | WHWLQLKPGQPMYGKTVLLRKLLKLLVRKI (SEQ ID | WKMGQYHQLPPLWKTVLLRKLLKLLVRKI |
| ID NO: 5827) | NO: 5826) | (SEQ ID NO: 5825) |
| GWHWLQLKPGQPMYGIGKFLHSAKKWGKAFVGQI | WHWLQLKPGQPMYGGIGKFLHSAKKWGKAFVGQIMN | WKMGQYHQLPPLWGIGKFLHSAKKWGKAF |
| MNC (SEQ ID NO: 5833) | C (SEQ ID NO: 5832) | VGQIMNC (SEQ ID NO: 5831) |

A CGI factor is a peptide that in embodiments includes an amino acid sequence that has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with at least one of SEQ ID NO: 961, SEQ ID NO: 962, SEQ ID NO: 963, SEQ ID NO: 964, SEQ ID NO: 965, SEQ ID NO: 966, SEQ ID NO: 967, SEQ ID NO: 968, SEQ ID NO: 969, SEQ ID NO: 970, SEQ ID NO: 971, SEQ ID NO: 972, SEQ ID NO: 973, SEQ ID NO: 974, SEQ ID NO: 975, SEQ ID NO: 976, SEQ ID NO: 977, SEQ ID NO: 978, SEQ ID NO: 979, SEQ ID NO: 980, SEQ ID NO: 981, SEQ ID NO: 982, SEQ ID NO: 983, SEQ ID NO: 984, SEQ ID NO: 985, SEQ ID NO: 986, SEQ ID NO: 987, SEQ ID NO: 988, SEQ ID NO: 989, SEQ ID NO: 990, SEQ ID NO: 991, SEQ ID NO: 992, SEQ ID NO: 993, SEQ ID NO: 994, SEQ ID NO: 995, SEQ ID NO: 996, SEQ ID NO: 997, SEQ ID NO: 998, SEQ ID NO: 999, SEQ ID NO: 1000, SEQ ID NO: 1001, SEQ ID NO: 1002, SEQ ID NO: 1003, SEQ ID NO: 1004, SEQ ID NO: 1005, SEQ ID NO: 1006, SEQ ID NO: 1007, SEQ ID NO: 1008, SEQ ID NO: 1009, SEQ ID NO: 1010, SEQ ID NO: 1011, SEQ ID NO: 1012, SEQ ID NO: 1013, SEQ ID NO: 1014, SEQ ID NO: 1015, SEQ ID NO: 1016, SEQ ID NO: 1017, SEQ ID NO: 1018, SEQ ID NO: 1019, SEQ ID NO: 1020, SEQ ID NO: 1021, SEQ ID NO: 1022, SEQ ID NO: 1023, SEQ ID NO: 1024, SEQ ID NO: 1025, SEQ ID NO: 1026, SEQ ID NO: 1027, SEQ ID NO: 1028, SEQ ID NO: 1029, SEQ ID NO: 1030, SEQ ID NO: 1031, SEQ ID NO: 1032, SEQ ID NO: 1033, SEQ ID NO: 1034, SEQ ID NO: 1035, SEQ ID NO: 1036, SEQ ID NO: 1037, SEQ ID NO: 1038, SEQ ID NO: 1039, SEQ ID NO: 1040, SEQ ID NO: 1041, SEQ ID NO: 1042, SEQ ID NO: 1043, SEQ ID NO: 1044, SEQ ID NO: 1045, SEQ ID NO: 1046, SEQ ID NO: 1047, SEQ ID NO: 1048, SEQ ID NO: 1049, SEQ ID NO: 1050, SEQ ID NO: 1051, SEQ ID NO: 1052, SEQ ID NO: 1053, SEQ ID NO: 1054, SEQ ID NO: 1055, SEQ ID NO: 1056, SEQ ID NO: 1057, SEQ ID NO: 1058, SEQ ID NO: 1059, SEQ ID NO: 1060, SEQ ID NO: 1061, SEQ ID NO: 1062, SEQ ID NO: 1063, SEQ ID NO: 1064, SEQ ID NO: 1065, SEQ ID NO: 1066, SEQ ID NO: 1067, SEQ ID NO: 1068, SEQ ID NO: 1069, SEQ ID NO: 1070, SEQ ID NO: 1071, SEQ ID NO: 1072, SEQ ID NO: 1073, SEQ ID NO: 1074, SEQ ID NO: 1075, SEQ ID NO: 1076, SEQ ID NO: 1077, SEQ ID NO: 1078, SEQ ID NO: 1079, SEQ ID NO: 1080, SEQ ID NO: 1081, SEQ ID NO: 1082, SEQ ID NO: 1083, SEQ ID NO: 1084, SEQ ID NO: 1085, SEQ ID NO: 1086, SEQ ID NO: 1087, SEQ ID NO: 1088, SEQ ID NO: 1089, SEQ ID NO: 1090, SEQ ID NO: 1091, SEQ ID NO: 1092, SEQ ID NO: 1093, SEQ ID NO: 1094, SEQ ID NO: 1095, SEQ ID NO: 1096, SEQ ID NO: 1097, SEQ ID NO: 1098, SEQ ID NO: 1099, SEQ ID NO: 1100, SEQ ID NO: 1101, SEQ ID NO: 1102, SEQ ID NO: 1103, SEQ ID NO: 1104, SEQ ID NO: 1105, SEQ ID NO: 1106, SEQ ID NO: 1107, SEQ ID NO: 1108, SEQ ID NO: 1109, SEQ ID NO: 1110, SEQ ID NO: 1111, SEQ ID NO: 1112, SEQ ID NO: 1113, SEQ ID NO: 1114, SEQ ID NO: 1115, SEQ ID NO: 1116, SEQ ID NO: 1117, SEQ ID NO: 1118, SEQ ID NO: 1119, SEQ ID NO: 1120, SEQ ID NO: 1121, SEQ ID NO: 1122, SEQ ID NO: 1123, SEQ ID NO: 1124, SEQ ID NO: 1125, SEQ ID NO: 1126, SEQ ID NO: 1127, SEQ ID NO: 1128, SEQ ID NO: 1129, SEQ ID NO: 1130, SEQ ID NO: 1131, SEQ ID NO: 1132, SEQ ID NO: 1133, SEQ ID NO: 1134, SEQ ID NO: 1135, SEQ ID NO: 1136, SEQ ID NO: 1137, SEQ ID NO: 1138, SEQ ID NO: 1139, SEQ ID NO: 1140, SEQ ID NO: 1141, SEQ ID NO: 1142, SEQ ID NO: 1143, SEQ ID NO: 1144, SEQ ID NO: 1145, SEQ ID NO: 1146, SEQ ID NO: 1147, SEQ ID NO: 1148, SEQ ID NO: 1149, SEQ ID NO: 1150, SEQ ID NO: 1151, SEQ ID NO: 1152, SEQ ID NO: 1153, SEQ ID NO: 1154, SEQ ID NO: 1155, SEQ ID NO: 1156, SEQ ID NO: 1157, SEQ ID NO: 1158, SEQ ID NO: 1159, SEQ ID NO: 1160, SEQ ID NO: 1161, SEQ ID NO: 1162, SEQ ID NO: 1163, SEQ ID NO: 1164, SEQ ID NO: 1165, SEQ ID NO: 1166, SEQ ID NO: 1167, SEQ ID NO: 1168, SEQ ID NO: 1169, SEQ ID NO: 1170, SEQ ID NO: 1171, SEQ ID NO: 1172, SEQ ID NO: 1173, SEQ ID NO: 1174, SEQ ID NO: 1175, SEQ ID NO: 1176, SEQ ID NO: 1177, SEQ ID NO: 1178, SEQ ID NO: 1179, SEQ ID NO: 1180, SEQ ID NO: 1181, SEQ ID NO: 1182, SEQ ID NO: 1183, SEQ ID NO: 1184, SEQ ID NO: 1185, SEQ ID NO: 1186, SEQ ID NO: 1187, SEQ ID NO: 1188, SEQ ID NO: 1189, SEQ ID NO: 1190, SEQ ID NO: 1191, SEQ ID NO: 1192, SEQ ID NO: 1193, SEQ ID NO: 1194, SEQ ID NO: 1195, SEQ ID NO: 1196, SEQ ID NO: 1197, SEQ ID NO: 1198, SEQ ID NO: 1199, SEQ ID NO: 1200, SEQ ID NO: 1201, SEQ ID NO: 1202, SEQ ID NO: 1203, SEQ ID NO: 1204, SEQ ID NO: 1205, SEQ ID NO: 1206, SEQ ID NO: 1207, SEQ ID NO: 1208, SEQ ID NO: 1209, SEQ ID NO: 1210, SEQ ID NO: 1211, SEQ ID NO: 1212, SEQ ID NO: 1213, SEQ ID NO: 1214, SEQ ID NO: 1215, SEQ ID NO: 1216, SEQ ID NO: 1217, SEQ ID NO: 1218, SEQ ID NO: 1219, SEQ ID NO: 1220, SEQ ID NO: 1221, SEQ ID NO: 1222, SEQ ID NO: 1223, SEQ ID NO: 1224, SEQ ID NO: 1225, SEQ ID NO: 1226, SEQ ID NO: 1227, SEQ ID NO: 1228, SEQ ID NO: 1229, SEQ ID NO: 1230, SEQ ID NO: 1231, SEQ ID NO: 1232, SEQ ID NO: 1233, SEQ ID NO: 1234, SEQ ID NO: 1235, SEQ ID NO: 1236, SEQ ID NO: 1237, SEQ ID NO: 1238, SEQ ID NO: 1239, SEQ ID NO: 1240, SEQ ID NO: 1241, SEQ ID NO: 1242, SEQ ID NO: 1243, SEQ ID NO: 1244, SEQ ID NO: 1245, SEQ ID NO: 1246, SEQ ID NO: 1247, SEQ ID NO: 1248, SEQ ID NO: 1249, SEQ ID NO: 1250, SEQ ID NO: 1251, SEQ ID NO: 1252, SEQ ID NO: 1253, SEQ ID NO: 1254, SEQ ID NO: 1255, SEQ ID NO: 1256, SEQ ID NO: 1257, SEQ ID NO: 1258, SEQ ID NO: 1259, SEQ ID NO: 1260, SEQ ID NO: 1261, SEQ ID NO: 1262, SEQ ID NO: 1263, SEQ ID NO: 1264, SEQ ID NO: 1265, SEQ ID NO: 1266, SEQ ID NO: 1267, SEQ ID NO: 1268, SEQ ID NO: 1269, SEQ ID NO: 1270, SEQ ID NO: 1271, SEQ ID NO: 1272, SEQ ID NO: 1273, SEQ ID NO: 1274, SEQ ID NO: 1275, SEQ ID NO: 1276, SEQ ID NO: 1277, SEQ ID NO: 1278, SEQ ID NO: 1279, SEQ ID NO: 1280, SEQ ID NO: 1281, SEQ ID NO: 1282, SEQ ID NO: 1283, SEQ ID NO: 1284, SEQ ID NO: 1285, SEQ ID NO: 1286, SEQ ID NO: 1287, SEQ ID NO: 1288, SEQ ID NO: 1289, SEQ ID NO: 1290, SEQ ID NO: 1291, SEQ ID NO: 1292, SEQ ID NO: 1293, SEQ ID NO: 1294, SEQ ID NO: 1295, SEQ ID NO: 1296, SEQ ID NO: 1297, SEQ ID NO: 1298, SEQ ID NO: 1299, SEQ ID NO: 1300, SEQ ID NO: 1301, SEQ ID NO: 1302, SEQ ID NO: 1303, SEQ ID NO: 1304, SEQ ID NO: 1305, SEQ ID NO: 1306, SEQ ID NO: 1307, SEQ ID NO: 1308, SEQ ID NO: 1309, SEQ ID NO: 1310, SEQ ID NO: 1311, SEQ ID NO: 1312, SEQ ID NO: 1313, SEQ ID NO: 1314, SEQ ID NO: 1315, SEQ ID NO: 1316, SEQ ID NO: 1317, SEQ ID NO: 1318, SEQ ID NO: 1319, SEQ ID NO: 1320, SEQ ID NO: 1321, SEQ ID NO: 1322, SEQ ID NO: 1323, SEQ ID NO: 1324, SEQ ID NO: 1325, SEQ ID NO: 1326, SEQ ID NO: 1327, SEQ ID NO: 1328, SEQ ID NO: 1329, SEQ ID NO: 1330, SEQ ID NO: 1331, SEQ ID NO: 1332, SEQ ID NO: 1333, SEQ ID NO: 1334, SEQ ID NO: 1335, SEQ ID NO: 1336, SEQ ID NO: 1337, SEQ ID NO: 1338, SEQ ID NO: 1339, SEQ ID NO: 1340, SEQ ID NO: 1341, SEQ ID NO: 1342, SEQ ID NO: 1343, SEQ ID NO: 1344, SEQ ID NO: 1345, SEQ ID NO: 1346, SEQ ID NO: 1347, SEQ ID NO: 1348, SEQ ID NO: 1349, SEQ ID NO: 1350, SEQ ID NO: 1351, SEQ ID NO: 1352, SEQ ID NO: 1353, SEQ ID NO: 1354, SEQ ID NO: 1355, SEQ ID NO: 1356, SEQ ID NO: 1357, SEQ ID NO: 1358, SEQ ID NO: 1359, SEQ ID NO: 1360, SEQ ID NO: 1361, SEQ ID NO: 1362, SEQ ID NO: 1363, SEQ ID NO: 1364, SEQ ID NO: 1365, SEQ ID NO: 1366, SEQ ID NO: 1367, SEQ ID NO: 1368, SEQ ID NO: 1369, SEQ ID NO: 1370, SEQ ID NO: 1371, SEQ ID NO: 1372, SEQ ID NO: 1373, SEQ ID NO: 1374, SEQ ID NO: 1375, SEQ ID NO: 1376, SEQ ID NO: 1377, SEQ ID NO: 1378, SEQ ID NO: 1379, SEQ ID NO: 1380, SEQ ID NO: 1381, SEQ ID NO: 1382, SEQ ID NO: 1383, SEQ ID NO: 1384, SEQ ID NO: 1385, SEQ ID NO: 1386, SEQ ID NO: 1387, SEQ ID NO: 1388, SEQ ID NO: 1389, SEQ ID NO: 1390, SEQ ID NO: 1391, SEQ ID NO: 1392, SEQ ID NO: 1393, SEQ ID NO: 1394, SEQ ID NO: 1395, SEQ ID NO: 1396, SEQ ID NO: 1397, SEQ ID NO: 1398, SEQ ID NO: 1399, SEQ ID NO: 1400, SEQ ID NO: 1401, SEQ ID NO: 1402, SEQ ID NO: 1403, SEQ ID NO: 1404, SEQ ID NO: 1405, SEQ ID NO: 1406, SEQ ID NO: 1407, SEQ ID NO: 1408, SEQ ID NO: 1409, SEQ ID NO: 1410, SEQ ID NO: 1411, SEQ ID NO: 1412, SEQ ID NO: 1413, SEQ ID NO: 1414, SEQ ID NO: 1415, SEQ ID NO: 1416, SEQ ID NO: 1417, SEQ ID NO: 1418, SEQ ID NO: 1419, SEQ ID NO: 1420, SEQ ID NO: 1421, SEQ ID NO: 1422, SEQ ID NO: 1423, SEQ ID NO: 1424, SEQ ID NO: 1425, SEQ ID NO: 1426, SEQ ID NO: 1427, SEQ ID NO: 1428, SEQ ID NO: 1429, SEQ ID NO: 1430, SEQ ID NO: 1431, SEQ ID NO: 1432, SEQ ID NO: 1433, SEQ ID NO: 1434, SEQ ID NO: 1435, SEQ ID NO: 1436, SEQ ID NO: 1437, SEQ ID NO: 1438, SEQ ID NO: 1439, SEQ ID NO: 1440, SEQ ID NO: 1441, SEQ ID NO: 1442, SEQ ID NO: 1443, SEQ ID NO: 1444, SEQ ID NO: 1445, SEQ ID NO: 1446, SEQ ID NO: 1447, SEQ ID NO: 1448, SEQ ID NO: 1449, SEQ ID NO: 1450, SEQ ID NO: 1451, SEQ ID NO: 1452, SEQ ID NO: 1453, SEQ ID NO: 1454, SEQ ID NO: 1455, SEQ ID NO: 1456, SEQ ID NO: 1457, SEQ ID NO: 1458, SEQ ID NO: 1459, SEQ ID NO: 1460, SEQ ID NO: 1461, SEQ ID NO: 1462, SEQ ID NO: 1463, SEQ ID NO: 1464, SEQ ID NO: 1465, SEQ ID NO: 1466, SEQ ID NO: 1467, SEQ ID NO: 1468, SEQ ID NO: 1469, SEQ ID NO: 1470, SEQ ID NO: 1471, SEQ ID NO: 1472, SEQ ID NO: 1473, SEQ ID NO: 1474, SEQ ID NO: 1475, SEQ ID NO: 1476, SEQ ID NO: 1477, SEQ ID NO: 1478, SEQ ID NO: 1479, SEQ ID NO: 1480, SEQ ID NO: 1481, SEQ ID NO: 1482, SEQ ID NO: 1483, SEQ ID NO: 1484, SEQ ID NO: 1485, SEQ ID NO: 1486, SEQ ID NO: 1487, SEQ ID NO: 1488, SEQ ID NO: 1489, SEQ ID NO: 1490, SEQ ID NO: 1491, SEQ ID NO: 1492, SEQ ID NO: 1493, SEQ ID NO: 1494, SEQ ID NO: 1495, SEQ ID NO: 1496, SEQ ID NO: 1497, SEQ ID NO: 1498, SEQ ID NO: 1499, SEQ ID NO: 1500, SEQ ID NO: 1501, SEQ ID NO: 1502, SEQ ID NO: 1503, SEQ ID NO: 1504, SEQ ID NO: 1505, SEQ ID NO: 1506, SEQ ID NO: 1507, SEQ ID NO: 1508, SEQ ID NO: 1509, SEQ ID NO: 1510, SEQ ID NO: 1511, SEQ ID NO: 1512, SEQ ID NO: 1513, SEQ ID NO: 1514, SEQ ID NO: 1515, SEQ ID NO: 1516, SEQ ID NO: 1517, SEQ ID NO: 1518, SEQ ID NO: 1519, SEQ ID NO: 1520, SEQ ID NO: 1521, SEQ ID NO: 1522, SEQ ID NO: 1523, SEQ ID NO: 1524, SEQ ID NO: 1525, SEQ ID NO: 1526, SEQ ID NO: 1527, SEQ ID NO: 1528, SEQ ID NO: 1529, SEQ ID NO: 1530, SEQ ID NO: 1531, SEQ ID NO: 1532, SEQ ID NO: 1533, SEQ ID NO: 1534, SEQ ID NO: 1535, SEQ ID NO: 1536, SEQ ID NO: 1537, SEQ ID NO: 1538, SEQ ID NO: 1539, SEQ ID NO: 1540, SEQ ID NO: 1541, SEQ ID NO: 1542, SEQ ID NO: 1543, SEQ ID NO: 1544, SEQ ID NO: 1545, SEQ ID NO: 1546, SEQ ID NO: 1547, SEQ ID NO: 1548, SEQ ID NO: 1549, SEQ ID NO: 1550, SEQ ID NO: 1551, SEQ ID NO: 1552, SEQ ID NO: 1553, SEQ ID NO: 1554, SEQ ID NO: 1555, SEQ ID NO: 1556, SEQ ID NO: 1557, SEQ ID NO: 1558, SEQ ID NO: 1559, SEQ ID NO: 1560, SEQ ID NO: 1561, SEQ ID NO: 1562, SEQ ID NO: 1563, SEQ ID NO: 1564, SEQ ID NO: 1565, SEQ ID NO: 1566, SEQ ID NO: 1567, SEQ ID NO: 1568, SEQ ID NO: 1569, SEQ ID NO: 1570, SEQ ID NO: 1571, SEQ ID NO: 1572, SEQ ID NO: 1573, SEQ ID NO: 1574, SEQ ID NO: 1575, SEQ ID NO: 1576, SEQ ID NO: 1577, SEQ ID NO: 1578, SEQ ID NO: 1579, SEQ ID NO: 1580, SEQ ID NO: 1581, SEQ ID NO: 1582, SEQ ID NO: 1583, SEQ ID NO: 1584, SEQ ID NO: 1585, SEQ ID NO: 1586, SEQ ID NO: 1587, SEQ ID NO: 1588, SEQ ID NO: 1589, SEQ ID NO: 1590, SEQ ID NO: 1591, SEQ ID NO: 1592, SEQ ID NO: 1593, SEQ ID NO: 1594, SEQ ID NO: 1595, SEQ ID NO: 1596, SEQ ID NO: 1597, SEQ ID NO: 1598, SEQ ID NO: 1599, SEQ ID NO: 1600, SEQ ID NO: 1601, SEQ ID NO: 1602, SEQ ID NO: 1603, SEQ ID NO: 1604, SEQ ID NO: 1605, SEQ ID NO: 1606, SEQ ID NO: 1607, SEQ ID NO: 1608, SEQ ID NO: 1609, SEQ ID NO: 1610, SEQ ID NO: 1611, SEQ ID NO: 1612, SEQ ID NO: 1613, SEQ ID NO: 1614, SEQ ID NO: 1615, SEQ ID NO: 1616, SEQ ID NO: 1617, SEQ ID NO: 1618, SEQ ID NO: 1619, SEQ ID NO: 1620, SEQ ID NO: 1621, SEQ ID NO: 1622, SEQ ID NO: 1623, SEQ ID NO: 1624, SEQ ID NO: 1625, SEQ ID NO: 1626, SEQ ID NO: 1627, SEQ ID NO: 1628, SEQ ID NO: 1629, SEQ ID NO: 1630, SEQ ID NO: 1631, SEQ ID NO: 1632, SEQ ID NO: 1633, SEQ ID NO: 1634, SEQ ID NO: 1635, SEQ ID NO: 1636, SEQ ID NO: 1637, SEQ ID NO: 1638, SEQ ID NO: 1639, SEQ ID NO: 1640, SEQ ID NO: 1641, SEQ ID NO: 1642, SEQ ID NO: 1643, SEQ ID NO: 1644, SEQ ID NO: 1645, SEQ ID NO: 1646, SEQ ID NO: 1647, SEQ ID NO: 1648, SEQ ID NO: 1649, SEQ ID NO: 1650, SEQ ID NO: 1651, SEQ ID NO: 1652, SEQ ID NO: 1653, SEQ ID NO: 1654, SEQ ID NO: 1655, SEQ ID NO: 1656, SEQ ID NO: 1657, SEQ ID NO: 1658, SEQ ID NO: 1659, SEQ ID NO: 1660, SEQ ID NO: 1661, SEQ ID NO: 1662, SEQ ID NO: 1663, SEQ ID NO: 1664, SEQ ID NO: 1665, SEQ ID NO: 1666, SEQ ID NO: 1667, SEQ ID NO: 1668, SEQ ID NO: 1669, SEQ ID NO: 1670, SEQ ID NO: 1671, SEQ ID NO: 1672, SEQ ID NO: 1673, SEQ ID NO: 1674, SEQ ID NO: 1675, SEQ ID NO: 1676, SEQ ID NO: 1677, SEQ ID NO: 1678, SEQ ID NO: 1679, SEQ ID NO: 1680, SEQ ID NO: 1681, SEQ ID NO: 1682, SEQ ID NO: 1683, SEQ ID NO: 1684, SEQ ID NO: 1685, SEQ ID NO: 1686, SEQ ID NO: 1687, SEQ ID NO: 1688, SEQ ID NO: 1689, SEQ ID NO: 1690, SEQ ID NO: 1691, SEQ ID NO: 1692, SEQ ID NO: 1693, SEQ ID NO: 1694, SEQ ID NO: 1695, SEQ ID NO: 1696, SEQ ID NO: 1697, SEQ ID NO: 1698, SEQ ID NO: 1699, SEQ ID NO: 1700, SEQ ID NO: 1701, SEQ ID NO: 1702, SEQ ID NO: 1703, SEQ ID NO: 1704, SEQ ID NO: 1705, SEQ ID NO: 1706, SEQ ID NO: 1707, SEQ ID NO: 1708, SEQ ID NO: 1709, SEQ ID NO: 1710, SEQ ID NO: 1711, SEQ ID NO: 1712, SEQ ID NO: 1713, SEQ ID NO: 1714, SEQ ID NO: 1715, SEQ ID NO: 1716, SEQ ID NO: 1717, SEQ ID NO: 1718, SEQ ID NO: 1719, SEQ ID NO: 1720, SEQ ID NO: 1721, SEQ ID NO: 1722, SEQ ID NO: 1723, SEQ ID NO: 1724, SEQ ID NO: 1725, SEQ ID NO: 1726, SEQ ID NO: 1727, SEQ ID NO: 1728, SEQ ID NO: 1729, SEQ ID NO: 1730, SEQ ID NO: 1731, SEQ ID NO: 1732, SEQ ID NO: 1733, SEQ ID NO: 1734, SEQ ID NO: 1735, SEQ ID NO: 1736, SEQ ID NO: 1737, SEQ ID NO: 1738, SEQ ID NO: 1739, SEQ ID NO: 1740, SEQ ID NO: 1741, SEQ ID NO: 1742, SEQ ID NO: 1743, SEQ ID NO: 1744, SEQ ID NO: 1745, SEQ ID NO: 1746, SEQ ID NO: 1747, SEQ ID NO: 1748, SEQ ID NO: 1749, SEQ ID NO: 1750, SEQ ID NO: 1751, SEQ ID NO: 1752, SEQ ID NO: 1753, SEQ ID NO: 1754, SEQ ID NO: 1755, SEQ ID NO: 1756, SEQ ID NO: 1757, SEQ ID NO: 1758, SEQ ID NO: 1759, SEQ ID NO: 1760, SEQ ID NO: 1761, SEQ ID NO: 1762, SEQ ID NO: 1763, SEQ ID NO: 1764, SEQ ID NO: 1765, SEQ ID NO: 1766, SEQ ID NO: 1767, SEQ ID NO: 1768, SEQ ID NO: 1769, SEQ ID NO: 1770, SEQ ID NO: 1771, SEQ ID NO: 1772, SEQ ID NO: 1773, SEQ ID NO: 1774, SEQ ID NO: 1775, SEQ ID NO: 1776, SEQ ID NO: 1777, SEQ ID NO: 1778, SEQ ID NO: 1779, SEQ ID NO: 1780, SEQ ID NO: 1781, SEQ ID NO: 1782, SEQ ID NO: 1783, SEQ ID NO: 1784, SEQ ID NO: 1785, SEQ ID NO: 1786, SEQ ID NO: 1787, SEQ ID NO: 1788, SEQ ID NO: 1789, SEQ ID NO: 1790, SEQ ID NO: 1791, SEQ ID NO: 1792, SEQ ID NO: 1793, SEQ ID NO: 1794, SEQ ID NO: 1795, SEQ ID NO: 1796, SEQ ID NO: 1797, SEQ ID NO: 1798, SEQ ID NO: 1799, SEQ ID NO: 1800, SEQ ID NO: 1801, SEQ ID NO: 1802, SEQ ID NO: 1803, SEQ ID NO: 1804, SEQ ID NO: 1805, SEQ ID NO: 1806, SEQ ID NO: 1807, SEQ ID NO: 1808, SEQ ID NO: 1809, SEQ ID NO: 1810, SEQ ID NO: 1811, SEQ ID NO: 1812, SEQ ID NO: 1813, SEQ ID NO: 1814, SEQ ID NO: 1815, SEQ ID NO: 1816, SEQ ID NO: 1817, SEQ ID NO: 1818, SEQ ID NO: 1819, SEQ ID NO: 1820, SEQ ID NO: 1821, SEQ ID NO: 1822, SEQ ID NO: 1823, SEQ ID NO: 1824, SEQ ID NO: 1825, SEQ ID NO: 1826, SEQ ID NO: 1827, SEQ ID NO: 1828, SEQ ID NO: 1829, SEQ ID NO: 1830, SEQ ID NO: 1831, SEQ ID NO: 1832, SEQ ID NO: 1833, SEQ ID NO: 1834, SEQ ID NO: 1835, SEQ ID NO: 1836, SEQ ID NO: 1837, SEQ ID NO: 1838, SEQ ID NO: 1839, SEQ ID NO: 1840, SEQ ID NO: 1841, SEQ ID NO: 1842, SEQ ID NO: 1843, SEQ ID NO: 1844, SEQ ID NO: 1845, SEQ ID NO: 1846, SEQ ID NO: 1847, SEQ ID NO: 1848, SEQ ID NO: 1849, SEQ ID NO: 1850, SEQ ID NO: 1851, SEQ ID NO: 1852, SEQ ID NO: 1853, SEQ ID NO: 1854, SEQ ID NO: 1855, SEQ ID NO: 1856, SEQ ID NO: 1857, SEQ ID NO: 1858, SEQ ID NO: 1859, SEQ ID NO: 1860, SEQ ID NO: 1861, SEQ ID NO: 1862, SEQ ID NO: 1863, SEQ ID NO: 1864, SEQ ID NO: 1865, SEQ ID NO: 1866, SEQ ID NO: 1867, SEQ ID NO: 1868, SEQ ID NO: 1869, SEQ ID NO: 1870, SEQ ID NO: 1871, SEQ ID NO: 1872, SEQ ID NO: 1873, SEQ ID NO: 1874, SEQ ID NO: 1875, SEQ ID NO: 1876, SEQ ID NO: 1877, SEQ ID NO: 1878, SEQ ID NO: 1879, SEQ ID NO: 1880, SEQ ID NO: 1881, SEQ ID NO: 1882, SEQ ID NO: 1883, SEQ ID NO: 1884, SEQ ID NO: 1885, SEQ ID NO: 1886, SEQ ID NO: 1887, SEQ ID NO: 1888, SEQ ID NO: 1889, SEQ ID NO: 1890, SEQ ID NO: 1891, SEQ ID NO: 1892, SEQ ID NO: 1893, SEQ ID NO: 1894, SEQ ID NO: 1895, SEQ ID NO: 1896, SEQ ID NO: 1897, SEQ ID NO: 1898, SEQ ID NO: 1899, SEQ ID NO: 1900, SEQ ID NO: 1901, SEQ ID NO: 1902, SEQ ID NO: 1903, SEQ ID NO: 1904, SEQ ID NO: 1905, SEQ ID NO: 1906, SEQ ID NO: 1907, SEQ ID NO: 1908, SEQ ID NO: 1909, SEQ ID NO: 1910, SEQ ID NO: 1911, SEQ ID NO: 1912, SEQ ID NO: 1913, SEQ ID NO: 1914, SEQ ID NO: 1915, SEQ ID NO: 1916, SEQ ID NO: 1917, SEQ ID NO: 1918, SEQ ID NO: 1919, SEQ ID NO: 1920, SEQ ID NO: 1957, SEQ ID NO: 1958, SEQ ID NO: 1959, SEQ ID NO: 1960, SEQ ID NO: 1961, SEQ ID NO: 1962, SEQ ID NO: 1963, SEQ ID NO: 1964, SEQ ID NO: 1965, SEQ ID NO: 1966, SEQ ID NO: 1967, SEQ ID NO: 1968, SEQ ID NO: 1969, SEQ ID NO: 1970, SEQ ID NO: 1971, SEQ ID NO: 1972, SEQ ID NO: 1973, SEQ ID NO: 1974, SEQ ID NO: 1975, SEQ ID NO: 1976, SEQ ID NO: 1977, SEQ ID NO: 1978, SEQ ID NO: 1979, SEQ ID NO: 1980, SEQ ID NO: 1981, SEQ ID NO: 1982, SEQ ID NO: 1983, SEQ ID NO: 1984, SEQ ID NO: 1985, SEQ ID NO: 1986, SEQ ID NO: 1987, SEQ ID NO: 1988, SEQ ID NO: 1989, SEQ ID NO: 1990, SEQ ID NO: 1991, SEQ ID NO: 1992, SEQ ID NO: 1993, SEQ ID NO: 1994, SEQ ID NO: 1995, SEQ ID NO: 1996, SEQ ID NO: 1997, SEQ ID NO: 1998, SEQ ID NO: 1999, SEQ ID NO: 2000, SEQ ID NO: 2001, SEQ ID NO: 2002, SEQ ID NO: 2003, SEQ ID NO: 2004, SEQ ID NO: 2005, SEQ ID NO: 2006, SEQ ID NO: 2007, SEQ ID NO: 2008, SEQ ID NO: 2009, SEQ ID NO: 2010, SEQ ID NO: 2011, SEQ ID NO: 2012, SEQ ID NO: 2013, SEQ ID NO: 2014, SEQ ID NO: 2015, SEQ ID NO: 2016, SEQ ID NO: 2017, SEQ ID NO: 2018, SEQ ID NO: 2019, SEQ ID NO: 2020, SEQ ID NO: 2021, SEQ ID NO: 2022, SEQ ID NO: 2023, SEQ ID NO: 2024, SEQ ID NO: 2025, SEQ ID NO: 2026, SEQ ID NO: 2027, SEQ ID NO: 2028, SEQ ID NO: 2029, SEQ ID NO: 2030, SEQ ID NO: 2031, SEQ ID NO: 2032, SEQ ID NO: 2033, SEQ ID NO: 2034, SEQ ID NO: 2035, SEQ ID NO: 2036, SEQ ID NO: 2037, SEQ ID NO: 2038, SEQ ID NO: 2039, SEQ ID NO: 2040, SEQ ID NO: 2041, SEQ ID NO: 2042, SEQ ID NO: 2043, SEQ ID NO: 2044, SEQ ID NO: 2045, SEQ ID NO: 2046, SEQ ID NO: 2047, SEQ ID NO: 2048, SEQ ID NO: 2049, SEQ ID NO: 2050, SEQ ID NO: 2051, SEQ ID NO: 2052, SEQ ID NO: 2053, SEQ ID NO: 2054, SEQ ID NO: 2055, SEQ ID NO: 2056, SEQ ID NO: 2057, SEQ ID NO: 2058, SEQ ID NO: 2059, SEQ ID NO: 2060, SEQ ID NO: 2061, SEQ ID NO: 2062, SEQ ID NO: 2063, SEQ ID NO: 2064, SEQ ID NO: 2065, SEQ ID NO: 2066, SEQ ID NO: 2067, SEQ ID NO: 2068, SEQ ID NO: 2069, SEQ ID NO: 2070, SEQ ID NO: 2071, SEQ ID NO: 2072, SEQ ID NO: 2073, SEQ ID NO: 2074, SEQ ID NO: 2075, SEQ ID NO: 2076, SEQ ID NO: 2077, SEQ ID NO: 2078, SEQ ID NO: 2079, SEQ ID NO: 2080, SEQ ID NO: 2081, SEQ ID NO: 2082, SEQ ID NO: 2083, SEQ ID NO: 2084, SEQ ID NO: 2085, SEQ ID NO: 2086, SEQ ID NO: 2087, SEQ ID NO: 2088, SEQ ID NO: 2089, SEQ ID NO: 2090, SEQ ID NO: 2091, SEQ ID NO: 2092, SEQ ID NO: 2093, SEQ ID NO: 2094, SEQ ID NO: 2095, SEQ ID NO: 2096, SEQ ID NO: 2097, SEQ ID NO: 2098, SEQ ID NO: 2099, SEQ ID NO: 2100, SEQ ID NO: 2101, SEQ ID NO: 2102, SEQ ID NO: 2103, SEQ ID NO: 2104, SEQ ID NO: 2105, SEQ ID NO: 2106, SEQ ID NO: 2107, SEQ ID NO: 2108, SEQ ID NO: 2109, SEQ ID NO: 2110, SEQ ID NO: 2111, SEQ ID NO: 2112, SEQ ID NO: 2113, SEQ ID NO: 2114, SEQ ID NO: 2115, SEQ ID NO: 2116, SEQ ID NO: 2117, SEQ ID NO: 2118, SEQ ID NO: 2119, SEQ ID NO: 2120, SEQ ID NO: 2121, SEQ ID NO: 2122, SEQ ID NO: 2123, SEQ ID NO: 2124, SEQ ID NO: 2125, SEQ ID NO: 2126, SEQ ID NO: 2127, SEQ ID NO: 2128, SEQ ID NO: 2129, SEQ ID NO: 2130, SEQ ID NO: 2131, SEQ ID NO: 2132, SEQ ID NO: 2133, SEQ ID NO: 2134, SEQ ID NO: 2135, SEQ ID NO: 2136, SEQ ID NO: 2137, SEQ ID NO: 2138, SEQ ID NO: 2139, SEQ ID NO: 2140, SEQ ID NO: 2141, SEQ ID NO: 2142, SEQ ID NO: 2143, SEQ ID NO: 2144, SEQ ID NO: 2145, SEQ ID NO: 2146, SEQ ID NO: 2147, SEQ ID NO: 2148, SEQ ID NO: 2149, SEQ ID NO: 2150, SEQ ID NO: 2151, SEQ ID NO: 2152, SEQ ID NO: 2153, SEQ ID NO: 2154, SEQ ID NO: 2155, SEQ ID NO: 2156, SEQ ID NO: 2157, SEQ ID NO: 2158, SEQ ID NO: 2159, SEQ ID NO: 2160, SEQ ID NO: 2161, SEQ ID NO: 2162, SEQ ID NO: 2163, SEQ ID NO: 2164, SEQ ID NO: 2165, SEQ ID NO: 2166, SEQ ID NO: 2167, SEQ ID NO: 2168, SEQ ID NO: 2169, SEQ ID NO: 2170, SEQ ID NO: 2171, SEQ ID NO: 2172, SEQ ID NO: 2173, SEQ ID NO: 2174, SEQ ID NO: 2175, SEQ ID NO: 2176, SEQ ID NO: 2177, SEQ ID NO: 2178, SEQ ID NO: 2179, SEQ ID NO: 2180, or SEQ ID NO: 2181. In additional embodiments, the CGI factor can include SEQ ID NO: 2182, SEQ ID NO: 2183, SEQ ID NO: 2184, SEQ ID NO: 2185, SEQ ID NO: 2186, SEQ ID NO: 2187, SEQ ID NO: 2188, SEQ ID NO: 2189, SEQ ID NO: 2194, SEQ ID NO: 2195, SEQ ID NO: 2196, SEQ ID NO: 2197, SEQ ID NO: 2198, SEQ ID NO: 2199, SEQ ID NO: 2200, SEQ ID NO: 2201, SEQ ID NO: 2202, SEQ ID NO: 2203, SEQ ID NO: 2204, SEQ ID NO: 2205, SEQ ID NO: 2206, SEQ ID NO: 2207, SEQ ID NO: 2208, SEQ ID NO: 2209, SEQ ID NO: 2210, SEQ ID NO: 2215, SEQ ID NO: 2216, SEQ ID NO: 2217, SEQ ID NO: 2218, SEQ ID NO: 2219, SEQ ID NO: 2220, SEQ ID NO: 2221, SEQ ID NO: 2222, SEQ ID NO: 2223, SEQ ID NO: 2224, SEQ ID NO: 2225, SEQ ID NO: 2226, SEQ ID NO: 2227, SEQ ID NO: 2228, SEQ ID NO: 2229, SEQ ID NO: 2230, SEQ ID NO: 2231, SEQ ID NO: 2232, SEQ ID NO: 2233, SEQ ID NO: 2234, SEQ ID NO: 2235, SEQ ID NO: 2236, SEQ ID NO: 2237, SEQ ID NO: 2238, SEQ ID NO: 2239, SEQ ID NO: 2240, SEQ ID NO: 2241, SEQ ID NO: 2242, or SEQ ID NO: 2243. In some embodiments, the CGI factor motif includes at least one of SEQ ID NO: 1921, SEQ ID NO: 1922, SEQ ID NO: 1923, SEQ ID NO: 1924, SEQ ID NO: 1925, SEQ ID NO: 1926, SEQ ID NO: 1927, SEQ ID NO: 1928, SEQ ID NO: 1929, SEQ ID NO: 1930, SEQ ID NO: 1931, SEQ ID NO: 1932, SEQ ID NO: 1933, SEQ ID NO: 1934, SEQ ID NO: 1935, SEQ ID NO: 1936, SEQ ID NO: 1937, SEQ ID NO: 1938, SEQ ID NO: 1939, SEQ ID NO: 1940, SEQ ID NO: 1941, SEQ ID NO: 1942, SEQ ID NO: 1943, SEQ ID NO: 1944, SEQ ID NO: 1945, SEQ ID NO: 1946, SEQ ID NO: 1947, SEQ ID NO: 1948, SEQ ID NO: 1949, SEQ ID NO: 1950, SEQ ID NO: 1951, SEQ ID NO: 1952, SEQ ID NO: 1953, SEQ ID NO: 1954, SEQ ID NO: 1955, or SEQ ID NO: 1956. In still further embodiments, the CGI factor can include SEQ ID NO: 2457, SEQ ID NO: 2458, SEQ ID NO: 2459, SEQ ID NO: 2460, SEQ ID NO: 2461, SEQ ID NO: 2462, SEQ ID NO: 2463, SEQ ID NO: 2464, SEQ ID NO: 2465, SEQ ID NO: 2466, SEQ ID NO: 2467, SEQ ID NO: 2468, SEQ ID NO: 2469, SEQ ID NO: 2470, SEQ ID NO: 2471, SEQ ID NO: 2472, SEQ ID NO: 2473, SEQ ID NO: 2474, SEQ ID NO: 2475, SEQ ID NO: 2476, SEQ ID NO: 2477, SEQ ID NO: 2478, SEQ ID NO: 2479, SEQ ID NO: 2480, SEQ ID NO: 2481, SEQ ID NO: 2482, SEQ ID NO: 2483, SEQ ID NO: 2484, SEQ ID NO: 2485, SEQ ID NO: 2486, SEQ ID NO: 2487, SEQ ID NO: 2488, SEQ ID NO: 2489, SEQ ID NO: 2490, SEQ ID NO: 2491, SEQ ID NO: 2492, SEQ ID NO: 2493, SEQ ID NO: 2494, SEQ ID NO: 2495, SEQ ID NO: 2496, SEQ ID NO: 2497, SEQ ID NO: 2498, SEQ ID NO: 2499, SEQ ID NO: 2500, SEQ ID NO: 2501, SEQ ID NO: 2502, SEQ ID NO: 2503, SEQ ID NO: 2504, SEQ ID NO: 2505, SEQ ID NO: 2506, SEQ ID NO: 2507, SEQ ID NO: 2508, SEQ ID NO: 2509, SEQ ID NO: 2510, SEQ ID NO: 2511, SEQ ID NO: 2512, SEQ ID NO: 2513, SEQ ID NO: 2514, SEQ ID NO: 2515, SEQ ID NO: 2516, SEQ ID NO: 2517, SEQ ID NO: 2518, SEQ ID NO: 2519, SEQ ID NO: 2520, SEQ ID NO: 2521, SEQ ID NO: 2522, SEQ ID NO: 2523, SEQ ID NO: 2524, SEQ ID NO: 2525, SEQ ID NO: 2526, SEQ ID NO: 2527, SEQ ID NO: 2528, SEQ ID NO: 2529, SEQ ID NO: 2530, SEQ ID NO: 2531, SEQ ID NO: 2532, SEQ ID NO: 2533, SEQ ID NO: 2534, SEQ ID NO: 2535, SEQ ID NO: 2536, SEQ ID NO: 2537, SEQ ID NO: 2538, SEQ ID NO: 2539, SEQ ID NO: 2540, SEQ ID NO: 2541, SEQ ID NO: 2542, SEQ ID NO: 2543, SEQ ID NO: 2544, SEQ ID NO: 2545, SEQ ID NO: 2546, SEQ ID NO: 2547, SEQ ID NO: 2548, SEQ ID NO: 2549, SEQ ID NO: 2550, SEQ ID NO: 2551, SEQ ID NO: 2552, SEQ ID NO: 2553, SEQ ID NO: 2554, SEQ ID NO: 2555, SEQ ID NO: 2556, SEQ ID NO: 2557, SEQ ID NO: 2558, SEQ ID NO: 2559, SEQ ID NO: 2560, SEQ ID NO: 2561, SEQ ID NO: 2562, SEQ ID NO: 2563, SEQ ID NO: 2564, SEQ ID NO: 2565, SEQ ID NO: 2566, SEQ ID NO: 2567, SEQ ID NO: 2568, SEQ ID NO: 2569, SEQ ID NO: 2570, SEQ ID NO: 2571, SEQ ID NO: 2572, SEQ ID NO: 2573, SEQ ID NO: 2574, SEQ ID NO: 2575, SEQ ID NO: 2576, SEQ ID NO: 2577, SEQ ID NO: 2578, SEQ ID NO: 2579, SEQ ID NO: 2580, SEQ ID NO: 2581, SEQ ID NO: 2582, SEQ ID NO: 2583, SEQ ID NO: 2584, SEQ ID NO: 2585, SEQ ID NO: 2586, SEQ ID NO: 2587, SEQ ID NO: 2588, SEQ ID NO: 2589, SEQ ID NO: 2590, SEQ ID NO: 2591, SEQ ID NO: 2592, SEQ ID NO: 2593, SEQ ID NO: 2594, SEQ ID NO: 2595, SEQ ID NO: 2596, SEQ ID NO: 2597, SEQ ID NO: 2598, SEQ ID NO: 2599, SEQ ID NO: 2600, SEQ ID NO: 2601, SEQ ID NO: 2602, SEQ ID NO: 2603, SEQ ID NO: 2604, SEQ ID NO: 2605, SEQ ID NO: 2606, SEQ ID NO: 2607, SEQ ID NO: 2608, SEQ ID NO: 2609, SEQ ID NO: 2610, SEQ ID NO: 2611, SEQ ID NO: 2612, SEQ ID NO: 2613, SEQ ID NO: 2614, SEQ ID NO: 2615, SEQ ID NO: 2616, SEQ ID NO: 2617, SEQ ID NO: 2618, SEQ ID NO: 2619, SEQ ID NO: 2620, SEQ ID NO: 2621, SEQ ID NO: 2622, SEQ ID NO: 2623, SEQ ID NO: 2624, SEQ ID NO: 2625, SEQ ID NO: 2626, SEQ ID NO: 2627, SEQ ID NO: 2628, SEQ ID NO: 2629, SEQ ID NO: 2630, SEQ ID NO: 2631, SEQ ID NO: 2632, SEQ ID NO: 2633, SEQ ID NO: 2634, SEQ ID NO: 2635, SEQ ID NO: 2636, SEQ ID NO: 2637, SEQ ID NO: 2638, SEQ ID NO: 2639, SEQ ID NO: 2640, SEQ ID NO: 2641, SEQ ID NO: 2642, SEQ ID NO: 2643, SEQ ID NO: 2644, SEQ ID NO: 2645, SEQ ID NO: 2646, SEQ ID NO: 2647, SEQ ID NO: 2648, SEQ ID NO: 2649, SEQ ID NO: 2650, SEQ ID NO: 2651, SEQ ID NO: 2652, SEQ ID NO: 2653, SEQ ID NO: 2654, SEQ ID NO: 2655, SEQ ID NO: 2656, SEQ ID NO: 2657, SEQ ID NO: 2658, SEQ ID NO: 2659, SEQ ID NO: 2660, SEQ ID NO: 2661, SEQ ID NO: 2662, SEQ ID NO: 2663, SEQ ID NO: 2664, SEQ ID NO: 2665, SEQ ID NO: 2666, SEQ ID NO: 2667, SEQ ID NO: 2668, SEQ ID NO: 2669, SEQ ID NO: 2670, SEQ ID NO: 2671, SEQ ID NO: 2672, SEQ ID NO: 2673, SEQ ID NO: 2674, SEQ ID NO: 2675, SEQ ID NO: 2676, SEQ ID NO: 2677, SEQ ID NO: 2678, SEQ ID NO: 2679, SEQ ID NO: 2680, SEQ ID NO: 2681, SEQ ID NO: 2682, SEQ ID NO: 2683, SEQ ID NO: 2684, SEQ ID NO: 2685, SEQ ID NO: 2686, SEQ ID NO: 2687, SEQ ID NO: 2688, SEQ ID NO: 2689, SEQ ID NO: 2690, SEQ ID NO: 2691, SEQ ID NO: 2692, SEQ ID NO: 2693, SEQ ID NO: 2694, SEQ ID NO: 2695, SEQ ID NO: 2696, SEQ ID NO: 2697, SEQ ID NO: 2698, SEQ ID NO: 2699, SEQ ID NO: 2700, SEQ ID NO: 2701, SEQ ID NO: 2702, SEQ ID NO: 2703, SEQ ID NO: 2704, SEQ ID NO: 2705, SEQ ID NO: 2706, SEQ ID NO: 2707, SEQ ID NO: 2708, SEQ ID NO: 2709, SEQ ID NO: 2710, SEQ ID NO: 2711, SEQ ID NO: 2712, SEQ ID NO: 2713, SEQ ID NO: 2714, SEQ ID NO: 2715, SEQ ID NO: 2716, SEQ ID NO: 2717, SEQ ID NO: 2718, SEQ ID NO: 2719, SEQ ID NO: 2720, SEQ ID NO: 2721, SEQ ID NO: 2722, SEQ ID NO: 2723, SEQ ID NO: 2724, SEQ ID NO: 2725, SEQ ID NO: 2726, SEQ ID NO: 2727, SEQ ID NO: 2728, SEQ ID NO: 2729, SEQ ID NO: 2730, SEQ ID NO: 2731, SEQ ID NO: 2732, SEQ ID NO: 2733, SEQ ID NO: 2734, SEQ ID NO: 2735, SEQ ID NO: 2736, SEQ ID NO: 2737, SEQ ID NO: 2738, SEQ ID NO: 2739, SEQ ID NO: 2740, SEQ ID NO: 2741, SEQ ID NO: 2742, SEQ ID NO: 2743, SEQ ID NO: 2744, SEQ ID NO: 2745, SEQ ID NO: 2746, SEQ ID NO: 2747, SEQ ID NO: 2748, SEQ ID NO: 2749, SEQ ID NO: 2750, SEQ ID NO: 2751, SEQ ID NO: 2752, SEQ ID NO: 2753, SEQ ID NO: 2754, SEQ ID NO: 2755, SEQ ID NO: 2756, SEQ ID NO: 2757, SEQ ID NO: 2758, SEQ ID NO: 2759, SEQ ID NO: 2760, SEQ ID NO: 2761, SEQ ID NO: 2762, SEQ ID NO: 2763, SEQ ID NO: 2764, SEQ ID NO: 2765, SEQ ID NO: 2766, SEQ ID NO: 2767, SEQ ID NO: 2768, SEQ ID NO: 2769, SEQ ID NO: 2770, SEQ ID NO: 2771, SEQ ID NO: 2772, SEQ ID NO: 2773, SEQ ID NO: 2774, SEQ ID NO: 2775, SEQ ID NO: 2776, SEQ ID NO: 2777, SEQ ID NO: 2778, SEQ ID NO: 2779, SEQ ID NO: 2780, SEQ ID NO: 2781, SEQ ID NO: 2782, SEQ ID NO: 2783, SEQ ID NO: 2784, SEQ ID NO: 2785, SEQ ID NO: 2786, SEQ ID NO: 2787, SEQ ID NO: 2788, SEQ ID NO: 2789, SEQ ID NO: 2790, SEQ ID NO: 2791, SEQ ID NO: 2792, SEQ ID NO: 2793, SEQ ID NO: 2794, SEQ ID NO: 2795, SEQ ID NO: 2796, SEQ ID NO: 2797, SEQ ID NO: 2798, SEQ ID NO: 2799, SEQ ID NO: 2800, SEQ ID NO: 2801, SEQ ID NO: 2802, SEQ ID NO: 2803, SEQ ID NO: 2804, SEQ ID NO: 2805, SEQ ID NO: 2806, SEQ ID NO: 2807, SEQ ID NO: 2808, SEQ ID NO: 2809, SEQ ID NO: 2810, SEQ ID NO: 2811, SEQ ID NO: 2812, SEQ ID NO: 2813, SEQ ID NO: 2814, SEQ ID NO: 2815, SEQ ID NO: 2816, SEQ ID NO: 2817, SEQ ID NO: 2818, SEQ ID NO: 2819, SEQ ID NO: 2820, SEQ ID NO: 2821, SEQ ID NO: 2822, SEQ ID NO: 2823, SEQ ID NO: 2824, SEQ ID NO: 2825, SEQ ID NO: 2826, SEQ ID NO: 2827, SEQ ID NO: 2828, SEQ ID NO: 2829, SEQ ID NO: 2830, SEQ ID NO: 2831, SEQ ID NO: 2832, SEQ ID NO: 2833, SEQ ID NO: 2834, SEQ ID NO: 2835, SEQ ID NO: 2836, SEQ ID NO: 2837, SEQ ID NO: 2838, SEQ ID NO: 2839, SEQ ID NO: 2840, SEQ ID NO: 2841, SEQ ID NO: 2842, SEQ ID NO: 2843, SEQ ID NO: 2844, SEQ ID NO: 2845, SEQ ID NO: 2846, SEQ ID NO: 2847, SEQ ID NO: 2848, SEQ ID NO: 2849, SEQ ID NO: 2850, SEQ ID NO: 2851, SEQ ID NO: 2852, SEQ ID NO: 2853, SEQ ID NO: 2854, SEQ ID NO: 2855, SEQ ID NO: 2856, SEQ ID NO: 2857, SEQ ID NO: 2858, SEQ ID NO: 2859, SEQ ID NO: 2860, SEQ ID NO: 2861, SEQ ID NO: 2862, SEQ ID NO: 2863, SEQ ID NO: 2864, SEQ ID NO: 2865, SEQ ID NO: 2866, SEQ ID NO: 2867, SEQ ID NO: 2868, SEQ ID NO: 2869, SEQ ID NO: 2870, SEQ ID NO: 2871, SEQ ID NO: 2872, SEQ ID NO: 2873, SEQ ID NO: 2874, SEQ ID NO: 2875, SEQ ID NO: 2876, SEQ ID NO: 2877, SEQ ID NO: 2878, SEQ ID NO: 2879, SEQ ID NO: 2880, SEQ ID NO: 2881, SEQ ID NO: 2882, SEQ ID NO: 2883, SEQ ID NO: 2884, SEQ ID NO: 2885, SEQ ID NO: 2886, SEQ ID NO: 2887, SEQ ID NO: 2888, SEQ ID NO: 2889, SEQ ID NO: 2890, SEQ ID NO: 2891, SEQ ID NO: 2892, SEQ ID NO: 2893, SEQ ID NO: 2894, SEQ ID NO: 2895, SEQ ID NO: 2896, SEQ ID NO: 2897, SEQ ID NO: 2898, SEQ ID NO: 2899, SEQ ID NO: 2900, SEQ ID NO: 2901, SEQ ID NO: 2902, SEQ ID NO: 2903, SEQ ID NO: 2904, SEQ ID NO: 2905, SEQ ID NO: 2906, SEQ ID NO: 2907, SEQ ID NO: 2908, SEQ ID NO: 2909, SEQ ID NO: 2910, SEQ ID NO: 2911, SEQ ID NO: 2912, SEQ ID NO: 2913, SEQ ID NO: 2914, SEQ ID NO: 2915, SEQ ID NO: 2916, SEQ ID NO: 2917, SEQ ID NO: 2918, SEQ ID NO: 2919, SEQ ID NO: 2920, SEQ ID NO: 2921, SEQ ID NO: 2922, SEQ ID NO: 2923, SEQ ID NO: 2924, SEQ ID NO: 2925, SEQ ID NO: 2926, SEQ ID NO: 2927, SEQ ID NO: 2928, SEQ ID NO: 2929, SEQ ID NO: 2930, SEQ ID NO: 2931, SEQ ID NO: 2932, SEQ ID NO: 2933, SEQ ID NO: 2934, SEQ ID NO: 2935, SEQ ID NO: 2936, SEQ ID NO: 2937, SEQ ID NO: 2938, SEQ ID NO: 2939, SEQ ID NO: 2940, SEQ ID NO: 2941, SEQ ID NO: 2942, SEQ ID NO: 2943, SEQ ID NO: 2944, SEQ ID NO: 2945, SEQ ID NO: 2946, SEQ ID NO: 2947, SEQ ID NO: 2948, SEQ ID NO: 2949, SEQ ID NO: 2950, SEQ ID NO: 2951, SEQ ID NO: 2952, SEQ ID NO: 2953, SEQ ID NO: 2954, SEQ ID NO: 2955, SEQ ID NO: 2956, SEQ ID NO: 2957, SEQ ID NO: 2958, SEQ ID NO: 2959, SEQ ID NO: 2960, SEQ ID NO: 2961, SEQ ID NO: 2962, SEQ ID NO: 2963, SEQ ID NO: 2964, SEQ ID NO: 2965, SEQ ID NO: 2966, SEQ ID NO: 2967, SEQ ID NO: 2968, SEQ ID NO: 2969, SEQ ID NO: 2970, SEQ ID NO: 2971, SEQ ID NO: 2972, SEQ ID NO: 2973, SEQ ID NO: 2974, SEQ ID NO: 2975, SEQ ID NO: 2976, SEQ ID NO: 2977, SEQ ID NO: 2978, SEQ ID NO: 2979, SEQ ID NO: 2980, SEQ ID NO: 2981, SEQ ID NO: 2982, SEQ ID NO: 2983, SEQ ID NO: 2984, SEQ ID NO: 2985, SEQ ID NO: 2986, SEQ ID NO: 2987, SEQ ID NO: 2988, SEQ ID NO: 2989, SEQ ID NO: 2990, SEQ ID NO: 2991, SEQ ID NO: 2992, SEQ ID NO: 2993, SEQ ID NO: 2994, SEQ ID NO: 2995, SEQ ID NO: 2996, SEQ ID NO: 2997, SEQ ID NO: 2998, SEQ ID NO: 2999, SEQ ID NO: 3000, SEQ ID NO: 3001, SEQ ID NO: 3002, SEQ ID NO: 3003, SEQ ID NO: 3004, SEQ ID NO: 3005, SEQ ID NO: 3006, SEQ ID NO: 3007, SEQ ID NO: 3008, SEQ ID NO: 3009, SEQ ID NO: 3010, SEQ ID NO: 3011, SEQ ID NO: 3012, SEQ ID NO: 3013, SEQ ID NO: 3014, SEQ ID NO: 3015, SEQ ID NO: 3016, SEQ ID NO: 3017, SEQ ID NO: 3018, SEQ ID NO: 3019, SEQ ID NO: 3020, SEQ ID NO: 3021, SEQ ID NO: 3022, SEQ ID NO: 3023, SEQ ID NO: 3024, SEQ ID NO: 3025, SEQ ID NO: 3026, SEQ ID NO: 3027, SEQ ID NO: 3028, SEQ ID NO: 3029, SEQ ID NO: 3030, SEQ ID NO: 3031, SEQ ID NO: 3032, SEQ ID NO: 3033, SEQ ID NO: 3034, SEQ ID NO: 3035, SEQ ID NO: 3036, SEQ ID NO: 3037, SEQ ID NO: 3038, SEQ ID NO: 3039, SEQ ID NO: 3040, SEQ ID NO: 3041, SEQ ID NO: 3042, SEQ ID NO: 3043, SEQ ID NO: 3044, SEQ ID NO: 3045, SEQ ID NO: 3046, SEQ ID NO: 3047, SEQ ID NO: 3048, SEQ ID NO: 3049, SEQ ID NO: 3050, SEQ ID NO: 3051, SEQ ID NO: 3052, SEQ ID NO: 3053, SEQ ID NO: 3054, SEQ ID NO: 3055, SEQ ID NO: 3056, SEQ ID NO: 3057, SEQ ID NO: 3058, SEQ ID NO: 3059, SEQ ID NO: 3060, SEQ ID NO: 3061, SEQ ID NO: 3062, SEQ ID NO: 3063, SEQ ID NO: 3064, SEQ ID NO: 3065, SEQ ID NO: 3066, SEQ ID NO: 3067, SEQ ID NO: 3068, SEQ ID NO: 3069, SEQ ID NO: 3070, SEQ ID NO: 3071, SEQ ID NO: 3072, SEQ ID NO: 3073, SEQ ID NO: 3074, SEQ ID NO: 3075, SEQ ID NO: 3076, SEQ ID NO: 3077, SEQ ID NO: 3078, SEQ ID NO: 3079, SEQ ID NO: 3080, SEQ ID NO: 3081, SEQ ID NO: 3082, SEQ ID NO: 3083, SEQ ID NO: 3084, SEQ ID NO: 3085, SEQ ID NO: 3086, SEQ ID NO: 3087, SEQ ID NO: 3088, SEQ ID NO: 3089, SEQ ID NO: 3090, SEQ ID NO: 3091, SEQ ID NO: 3092, SEQ ID NO: 3093, SEQ ID NO: 3094, SEQ ID NO: 3095, SEQ ID NO: 3096, SEQ ID NO: 3097, SEQ ID NO: 3098, SEQ ID NO: 3099, SEQ ID NO: 3100, SEQ ID NO: 3101, SEQ ID NO: 3102, SEQ ID NO: 3103, SEQ ID NO: 3104, SEQ ID NO: 3105, SEQ ID NO: 3106, SEQ ID NO: 3107, SEQ ID NO: 3108, SEQ ID NO: 3109, SEQ ID NO: 3110, SEQ ID NO: 3111, SEQ ID NO: 3112, SEQ ID NO: 3113, SEQ ID NO: 3114, SEQ ID NO: 3115, SEQ ID NO: 3116, SEQ ID NO: 3117, SEQ ID NO: 3118, SEQ ID NO: 3119, SEQ ID NO: 3120, SEQ ID NO: 3121, SEQ ID NO: 3122, SEQ ID NO: 3123, SEQ ID NO: 3124, SEQ ID NO: 3125, SEQ ID NO: 3126, SEQ ID NO: 3127, SEQ ID NO: 3128, SEQ ID NO: 3129, SEQ ID NO: 3130, SEQ ID NO: 3131, SEQ ID NO: 3132, SEQ ID NO: 3133, SEQ ID NO: 3134, SEQ ID NO: 3135, SEQ ID NO: 3136, SEQ ID NO: 3137, SEQ ID NO: 3138, SEQ ID NO: 3139, SEQ ID NO: 3140, SEQ ID NO: 3141, SEQ ID NO: 3142, SEQ ID NO: 3143, SEQ ID NO: 3144, SEQ ID NO: 3145, SEQ ID NO: 3146, SEQ ID NO: 3147, SEQ ID NO: 3148, SEQ ID NO: 3149, SEQ ID NO: 3150, SEQ ID NO: 3151, SEQ ID NO: 3152, SEQ ID NO: 3153, SEQ ID NO: 3154, SEQ ID NO: 3155, SEQ ID NO: 3156, SEQ ID NO: 3157, SEQ ID NO: 3158, SEQ ID NO: 3159, SEQ ID NO: 3160, SEQ ID NO: 3161, SEQ ID NO: 3162, SEQ ID NO: 3163, SEQ ID NO: 3164, SEQ ID NO: 3165, SEQ ID NO: 3166, SEQ ID NO: 3167, SEQ ID NO: 3168, SEQ ID NO: 3169, SEQ ID NO: 3170, SEQ ID NO: 3171, SEQ ID NO: 3172, SEQ ID NO: 3173, SEQ ID NO: 3174, SEQ ID NO: 3175, SEQ ID NO: 3176, SEQ ID NO: 3177, SEQ ID NO: 3178, SEQ ID NO: 3179, SEQ ID NO: 3180, SEQ ID NO: 3181, SEQ ID NO: 3182, SEQ ID NO: 3183, SEQ ID NO: 3184, SEQ ID NO: 3185, SEQ ID NO: 3186, SEQ ID NO: 3187, SEQ ID NO: 3188, SEQ ID NO: 3189, SEQ ID NO: 3190, SEQ ID NO: 3191, SEQ ID NO: 3192, SEQ ID NO: 3193, SEQ ID NO: 3194, SEQ ID NO: 3195, SEQ ID NO: 3196, SEQ ID NO: 3197, SEQ ID NO: 3198, SEQ ID NO: 3199, SEQ ID NO: 3200, SEQ ID NO: 3201, SEQ ID NO: 3202, SEQ ID NO: 3203, SEQ ID NO: 3204, SEQ ID NO: 3205, SEQ ID NO: 3206, SEQ ID NO: 3207, SEQ ID NO: 3208, SEQ ID NO: 3209, SEQ ID NO: 3210, SEQ ID NO: 3211, SEQ ID NO: 3212, SEQ ID NO: 3213, SEQ ID NO: 3214, SEQ ID NO: 3215, SEQ ID NO: 3216, SEQ ID NO: 3217, SEQ ID NO: 3218, SEQ ID NO: 3219, SEQ ID NO: 3220, SEQ ID NO: 3221, SEQ ID NO: 3222, SEQ ID NO: 3223, SEQ ID NO: 3224, SEQ ID NO: 3225, SEQ ID NO: 3226, SEQ ID NO: 3227, SEQ ID NO: 3228, SEQ ID NO: 3229, SEQ ID NO: 3230, SEQ ID NO: 3231, SEQ ID NO: 3232, SEQ ID NO: 3233, SEQ ID NO: 3234, SEQ ID NO: 3235, SEQ ID NO: 3236, SEQ ID NO: 3237, SEQ ID NO: 3238, SEQ ID NO: 3239, SEQ ID NO: 3240, SEQ ID NO: 3241, SEQ ID NO: 3242, SEQ ID NO: 3243, SEQ ID NO: 3244, SEQ ID NO: 3245, SEQ ID NO: 3246, SEQ ID NO: 3247, SEQ ID NO: 3248, SEQ ID NO: 3249, SEQ ID NO: 3250, SEQ ID NO: 3251, SEQ ID NO: 3252, SEQ ID NO: 3253, SEQ ID NO: 3254, SEQ ID NO: 3255, SEQ ID NO: 3256, SEQ ID NO: 3257, SEQ ID NO: 3258, SEQ ID NO: 3259, SEQ ID NO: 3260, SEQ ID NO: 3261, SEQ ID NO: 3262, SEQ ID NO: 3263, SEQ ID NO: 3264, SEQ ID NO: 3265, SEQ ID NO: 3266, SEQ ID NO: 3267, SEQ ID NO: 3268, SEQ ID NO: 3269, SEQ ID NO: 3270, SEQ ID NO: 3271, SEQ ID NO: 3272, SEQ ID NO: 3273, SEQ ID NO: 3274, SEQ ID NO: 3275, SEQ ID NO: 3276, SEQ ID NO: 3277, SEQ ID NO: 3278, SEQ ID NO: 3279, SEQ ID NO: 3280, SEQ ID NO: 3281, SEQ ID NO: 3282, SEQ ID NO: 3283, SEQ ID NO: 3284, SEQ ID NO: 3285, SEQ ID NO: 3286, SEQ ID NO: 3287, SEQ ID NO: 3288, SEQ ID NO: 3289, SEQ ID NO: 3290, SEQ ID NO: 3291, SEQ ID NO: 3292, SEQ ID NO: 3293, SEQ ID NO: 3294, SEQ ID NO: 3295, SEQ ID NO: 3296, SEQ ID NO: 3297, SEQ ID NO: 3298, SEQ ID NO: 3299, SEQ ID NO: 3300, SEQ ID NO: 3301, SEQ ID NO: 3302, SEQ ID NO: 3303, SEQ ID NO: 3304, SEQ ID NO: 3305, SEQ ID NO: 3306, SEQ ID NO: 3307, SEQ ID NO: 3308, SEQ ID NO: 3309, SEQ ID NO: 3310, SEQ ID NO: 3311, SEQ ID NO: 3312, SEQ ID NO: 3313, SEQ ID NO: 3314, SEQ ID NO: 3315, SEQ ID NO: 3316, SEQ ID NO: 3317, SEQ ID NO: 3318, SEQ ID NO: 3319, SEQ ID NO: 3320, SEQ ID NO: 3321, SEQ ID NO: 3322, SEQ ID NO: 3323, SEQ ID NO: 3324, SEQ ID NO: 3325, SEQ ID NO: 3326, SEQ ID NO: 3327, SEQ ID NO: 3328, SEQ ID NO: 3329, SEQ ID NO: 3330, SEQ ID NO: 3331, SEQ ID NO: 3332, SEQ ID NO: 3333, SEQ ID NO: 3334, SEQ ID NO: 3335, SEQ ID NO: 3336, SEQ ID NO: 3337, SEQ ID NO: 3338, SEQ ID NO: 3339, SEQ ID NO: 3340, SEQ ID NO: 3341, SEQ ID NO: 3342, SEQ ID NO: 3343, SEQ ID NO: 3344, SEQ ID NO: 3345, SEQ ID NO: 3346, SEQ ID NO: 3347, SEQ ID NO: 3348, SEQ ID NO: 3349, SEQ ID NO: 3350, SEQ ID NO: 3351, SEQ ID NO: 3352, SEQ ID NO: 3353, SEQ ID NO: 3354, SEQ ID NO: 3355, SEQ ID NO: 3356, SEQ ID NO: 3357, SEQ ID NO: 3358, SEQ ID NO: 3359, SEQ ID NO: 3360, or SEQ ID NO: 3361. In yet additional embodiments, the CGI factor can include SEQ ID NO: 5707, SEQ ID NO: 5708, SEQ ID NO: 5709, SEQ ID NO: 5710, SEQ ID NO: 5711, SEQ ID NO: 5712, SEQ ID NO: 5713, SEQ ID NO: 5714, SEQ ID NO: 5715, SEQ ID NO: 5716, SEQ ID NO: 5717, SEQ ID NO: 5718, SEQ ID NO: 5719, SEQ ID NO: 5720, SEQ ID NO: 5721, SEQ ID NO: 5722, SEQ ID NO: 5723, SEQ ID NO: 5724, SEQ ID NO: 5725, SEQ ID NO: 5726, SEQ ID NO: 5727, SEQ ID NO: 5728, SEQ ID NO: 5729, SEQ ID NO: 5730, SEQ ID NO: 5731, SEQ ID NO: 5755, SEQ ID NO: 5756, SEQ ID NO: 5757, SEQ ID NO: 5758, SEQ ID NO: 5759, SEQ ID NO: 5760, SEQ ID NO: 5761, SEQ ID NO: 5762, SEQ ID NO: 5763, SEQ ID NO: 5764, SEQ ID NO: 5765, SEQ ID NO: 5766, SEQ ID NO: 5767, SEQ ID NO: 5768, SEQ ID NO: 5769, SEQ ID NO: 5770, SEQ ID NO: 5771, SEQ ID NO: 5772, SEQ ID NO: 5773, SEQ ID NO: 5774, SEQ ID NO: 5775, SEQ ID NO: 5776, SEQ ID NO: 5777, SEQ ID NO: 5778, SEQ ID NO: 5779, SEQ ID NO: 5780, SEQ ID NO: 5781, SEQ ID NO: 5782, SEQ ID NO: 5783, SEQ ID NO: 5784, SEQ ID NO: 5785, SEQ ID NO: 5786, SEQ ID NO: 5787, SEQ ID NO: 5788, SEQ ID NO: 5789, SEQ ID NO: 5790, SEQ ID NO: 5791, SEQ ID NO: 5792, SEQ ID NO: 5793, SEQ ID NO: 5794, SEQ ID NO: 5795, SEQ ID NO: 5796, SEQ ID NO: 5797, SEQ ID NO: 5798, SEQ ID NO: 5799, SEQ ID NO: 5800, SEQ ID NO: 5801, SEQ ID NO: 5802, SEQ ID NO: 5803, SEQ ID NO: 5804, SEQ ID NO: 5805, SEQ ID NO: 5806, SEQ ID NO: 5807, SEQ ID NO: 5808, SEQ ID NO: 5809, SEQ ID NO: 5810, SEQ ID NO: 5811, SEQ ID NO: 5812, SEQ ID NO: 5813, SEQ ID NO: 5814, SEQ ID NO: 5815, SEQ ID NO: 5816, SEQ ID NO: 5817, SEQ ID NO: 5818, SEQ ID NO: 5819, SEQ ID NO: 5820, SEQ ID NO: 5821, SEQ ID NO: 5822, SEQ ID NO: 5823, SEQ ID NO: 5824, SEQ ID NO: 5825, SEQ ID NO: 5826, SEQ ID NO: 5827, SEQ ID NO: 5828, SEQ ID NO: 5829, SEQ ID NO: 5830, SEQ ID NO: 5831, SEQ ID NO: 5832, SEQ ID NO: 5833, SEQ ID NO: 5834, SEQ ID NO: 5835, SEQ ID NO: 5836, SEQ ID NO: 5837, SEQ ID NO: 5838, SEQ ID NO: 5839, SEQ ID NO: 5840, SEQ ID NO: 5841, SEQ ID NO: 5842, SEQ ID NO: 5843, SEQ ID NO: 5844, SEQ ID NO: 5845, SEQ ID NO: 5846, SEQ ID NO: 5847, SEQ ID NO: 5848, SEQ ID NO: 5849, SEQ ID NO: 5850, SEQ ID NO: 5851, SEQ ID NO: 5852, SEQ ID NO: 5853, SEQ ID NO: 5854, SEQ ID NO: 5855, SEQ ID NO: 5856, SEQ ID NO: 5857, SEQ ID NO: 5858, SEQ ID NO: 5859, SEQ ID NO: 5860, SEQ ID NO: 5861, SEQ ID NO: 5862, SEQ ID NO: 5863, SEQ ID NO: 5864, SEQ ID NO: 5865, SEQ ID NO: 5866, SEQ ID NO: 5867, SEQ ID NO: 5868, SEQ ID NO: 5869, SEQ ID NO: 5870, SEQ ID NO: 5871, SEQ ID NO: 5872, SEQ ID NO: 5873, SEQ ID NO: 5874, SEQ ID NO: 5875, SEQ ID NO: 5876, SEQ ID NO: 5877, SEQ ID NO: 5878, SEQ ID NO: 5879, SEQ ID NO: 5880, SEQ ID NO: 5881, SEQ ID NO: 5882, SEQ ID NO: 5883, SEQ ID NO: 5884, SEQ ID NO: 5885, SEQ ID NO: 5886, SEQ ID NO: 5887, SEQ ID NO: 5888, SEQ ID NO: 5889, SEQ ID NO: 5890, SEQ ID NO: 5891, SEQ ID NO: 5892, SEQ ID NO: 5893, SEQ ID NO: 5894, SEQ ID NO: 5895, SEQ ID NO: 5896, SEQ ID NO: 5897, SEQ ID NO: 5898, SEQ ID NO: 5899, SEQ ID NO: 5900, SEQ ID NO: 5901, SEQ ID NO: 5902, SEQ ID NO: 5903, SEQ ID NO: 5904, SEQ ID NO: 5905, SEQ ID NO: 5906, SEQ ID NO: 5907, SEQ ID NO: 5908, SEQ ID NO: 5909, SEQ ID NO: 5910, or SEQ ID NO: 5911.

A “CGI factor fragment” refers to an amino acid sequence that is reduced by one amino acid or two amino acids relative to a CGI factor, while retaining the function of the CGI factor. A “CGI factor precursor” is a polypeptide that includes at least one copy of a CGI factor (in embodiments, two or more copies of a CGI factor or multiple different CGI factors) and that is processed to the mature CGI factor(s), e.g., through proteolytic cleavage. CGI factor precursors include precursors with sequences encoded natively in one or more fungal genomes, as well as precursors having synthetic sequences. An “active” CGI factor, CGI factor precursor, CGI factor fragment, or CGI factor motif refers to a CGI factor, CGI factor precursor, CGI factor fragment, or CGI factor motif that is able to inhibit fungal conidial germination activity (conidial germination inhibitory), fungal growth, or fungal reproduction. A “toxic” CGI factor, CGI factor precursor, CGI factor fragment, or CGI factor motif refers to a CGI factor, CGI factor precursor, CGI factor fragment, or CGI factor motif that is able to kill a fungus or that decreases the number of viable cells in a population of fungal cells (i.e., is fungicidal). In embodiments, a CGI factor, CGI factor precursor, or CGI factor fragment is active without being toxic, or is toxic without being active, or is both active and toxic. Both active and/or toxic CGI factors, CGI factor precursors, or CGI factor fragments are CGI factors, CGI factor precursors, or CGI factor fragments of the present disclosure.

The CGI factor motif includes the sequence WX₁WX₂X₃X₄X₅X₆GX₇PXSY (SEQ ID NO: 1921). In this sequence, X₁ is selected from the group of T, E, K, Q, S, R, G and H; X₂ is selected from the group of G, I, V and L; X₃ is selected from the group of A, T, K, E, N, S, R and Q; X₄ is selected from the group of I, F and L; X₅ is selected from the group of E, A, Y, Q, M, F, S, G, D, R and K; X₆ is selected from the group of N, T, L, A, Y, I, W, V, M, K, R and P; X₇ is selected from the group of A, E, and Q; and X₈ is selected from the group of F, L, I, and M. The CGI factor motif can be SEQ ID NO: 1922, SEQ ID NO: 1923, SEQ ID NO: 1924, SEQ ID NO: 1925, SEQ ID NO: 1926, SEQ ID NO: 1927, SEQ ID NO: 1928, SEQ ID NO: 1929, SEQ ID NO: 1930, SEQ ID NO: 1931, SEQ ID NO: 1932, SEQ ID NO: 1933, SEQ ID NO: 1934, SEQ ID NO: 1935, SEQ ID NO: 1936, SEQ ID NO: 1937, SEQ ID NO: 1938, SEQ ID NO: 1939, SEQ ID NO: 1940, SEQ ID NO: 1941, SEQ ID NO: 1942, SEQ ID NO: 1943, SEQ ID NO: 1944, SEQ ID NO: 1945, SEQ ID NO: 1946, SEQ ID NO: 1947, SEQ ID NO: 1948, SEQ ID NO: 1949, SEQ ID NO: 1950, SEQ ID NO: 1951, SEQ ID NO: 1952, SEQ ID NO: 1953, SEQ ID NO: 1954, SEQ ID NO: 1955, or SEQ ID NO: 1956, each of which represents a motif subset of the sequence WX₁WX₂X₃X₄X₅X₆GX₇PXSY (SEQ ID NO: 1921).

### Recombinant DNA Constructs and Vectors

Recombinant DNA constructs and vectors can be used for production of CGI factors, CGI factor precursors, CGI factor motifs, or CGI factor fragments of the disclosure and the generation of transgenic cells and organisms. A recombinant DNA construct for use with the disclosure includes a heterologous promoter operably linked to a nucleic acid molecule including a nucleotide sequence that encodes (a) the amino acid sequence of at least one CGI factor, CGI factor precursor, or CGI factor fragment described herein; (b) a synthetic sequence of (a) that has codons optimized for heterologous expression; or (c) at least one CGI factor motif. The nucleotide sequence encoding the at least one CGI factor of the preceding embodiment can include SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330, SEQ ID NO: 331, SEQ ID NO: 332, SEQ ID NO: 333, SEQ ID NO: 334, SEQ ID NO: 335, SEQ ID NO: 336, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, SEQ ID NO: 376, SEQ ID NO: 377, SEQ ID NO: 378, SEQ ID NO: 379, SEQ ID NO: 380, SEQ ID NO: 381, SEQ ID NO: 382, SEQ ID NO: 383, SEQ ID NO: 384, SEQ ID NO: 385, SEQ ID NO: 386, SEQ ID NO: 387, SEQ ID NO: 388, SEQ ID NO: 389, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 402, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, SEQ ID NO: 446, SEQ ID NO: 447, SEQ ID NO: 448, SEQ ID NO: 449, SEQ ID NO: 450, SEQ ID NO: 451, SEQ ID NO: 452, SEQ ID NO: 453, SEQ ID NO: 454, SEQ ID NO: 455, SEQ ID NO: 456, SEQ ID NO: 457, SEQ ID NO: 458, SEQ ID NO: 459, SEQ ID NO: 460, SEQ ID NO: 461, SEQ ID NO: 462, SEQ ID NO: 463, SEQ ID NO: 464, SEQ ID NO: 465, SEQ ID NO: 466, SEQ ID NO: 467, SEQ ID NO: 468, SEQ ID NO: 469, SEQ ID NO: 470, SEQ ID NO: 471, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, SEQ ID NO: 512, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 523, SEQ ID NO: 524, SEQ ID NO: 525, SEQ ID NO: 526, SEQ ID NO: 527, SEQ ID NO: 528, SEQ ID NO: 529, SEQ ID NO: 530, SEQ ID NO: 531, SEQ ID NO: 532, SEQ ID NO: 533, SEQ ID NO: 534, SEQ ID NO: 535, SEQ ID NO: 536, SEQ ID NO: 537, SEQ ID NO: 538, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, SEQ ID NO: 575, SEQ ID NO: 576, SEQ ID NO: 577, SEQ ID NO: 578, SEQ ID NO: 579, SEQ ID NO: 580, SEQ ID NO: 581, SEQ ID NO: 582, SEQ ID NO: 583, SEQ ID NO: 584, SEQ ID NO: 585, SEQ ID NO: 586, SEQ ID NO: 587, SEQ ID NO: 588, SEQ ID NO: 589, SEQ ID NO: 590, SEQ ID NO: 591, SEQ ID NO: 592, SEQ ID NO: 593, SEQ ID NO: 594, SEQ ID NO: 595, SEQ ID NO: 596, SEQ ID NO: 597, SEQ ID NO: 598, SEQ ID NO: 599, SEQ ID NO: 600, SEQ ID NO: 601, SEQ ID NO: 602, SEQ ID NO: 603, SEQ ID NO: 604, SEQ ID NO: 605, SEQ ID NO: 606, SEQ ID NO: 607, SEQ ID NO: 608, SEQ ID NO: 609, SEQ ID NO: 610, SEQ ID NO: 611, SEQ ID NO: 612, SEQ ID NO: 613, SEQ ID NO: 614, SEQ ID NO: 615, SEQ ID NO: 616, SEQ ID NO: 617, SEQ ID NO: 618, SEQ ID NO: 619, SEQ ID NO: 620, SEQ ID NO: 621, SEQ ID NO: 622, SEQ ID NO: 623, SEQ ID NO: 624, SEQ ID NO: 625, SEQ ID NO: 626, SEQ ID NO: 627, SEQ ID NO: 628, SEQ ID NO: 629, SEQ ID NO: 630, SEQ ID NO: 631, SEQ ID NO: 632, SEQ ID NO: 633, SEQ ID NO: 634, SEQ ID NO: 635, SEQ ID NO: 636, SEQ ID NO: 637, SEQ ID NO: 638, SEQ ID NO: 639, SEQ ID NO: 640, SEQ ID NO: 641, SEQ ID NO: 642, SEQ ID NO: 643, SEQ ID NO: 644, SEQ ID NO: 645, SEQ ID NO: 646, SEQ ID NO: 647, SEQ ID NO: 648, SEQ ID NO: 649, SEQ ID NO: 650, SEQ ID NO: 651, SEQ ID NO: 652, SEQ ID NO: 653, SEQ ID NO: 654, SEQ ID NO: 655, SEQ ID NO: 656, SEQ ID NO: 657, SEQ ID NO: 658, SEQ ID NO: 659, SEQ ID NO: 660, SEQ ID NO: 661, SEQ ID NO: 662, SEQ ID NO: 663, SEQ ID NO: 664, SEQ ID NO: 665, SEQ ID NO: 666, SEQ ID NO: 667, SEQ ID NO: 668, SEQ ID NO: 669, SEQ ID NO: 670, SEQ ID NO: 671, SEQ ID NO: 672, SEQ ID NO: 673, SEQ ID NO: 674, SEQ ID NO: 675, SEQ ID NO: 676, SEQ ID NO: 677, SEQ ID NO: 678, SEQ ID NO: 679, SEQ ID NO: 680, SEQ ID NO: 681, SEQ ID NO: 682, SEQ ID NO: 683, SEQ ID NO: 684, SEQ ID NO: 685, SEQ ID NO: 686, SEQ ID NO: 687, SEQ ID NO: 688, SEQ ID NO: 689, SEQ ID NO: 690, SEQ ID NO: 691, SEQ ID NO: 692, SEQ ID NO: 693, SEQ ID NO: 694, SEQ ID NO: 695, SEQ ID NO: 696, SEQ ID NO: 697, SEQ ID NO: 698, SEQ ID NO: 699, SEQ ID NO: 700, SEQ ID NO: 701, SEQ ID NO: 702, SEQ ID NO: 703, SEQ ID NO: 704, SEQ ID NO: 705, SEQ ID NO: 706, SEQ ID NO: 707, SEQ ID NO: 708, SEQ ID NO: 709, SEQ ID NO: 710, SEQ ID NO: 711, SEQ ID NO: 712, SEQ ID NO: 713, SEQ ID NO: 714, SEQ ID NO: 715, SEQ ID NO: 716, SEQ ID NO: 717, SEQ ID NO: 718, SEQ ID NO: 719, SEQ ID NO: 720, SEQ ID NO: 721, SEQ ID NO: 722, SEQ ID NO: 723, SEQ ID NO: 724, SEQ ID NO: 725, SEQ ID NO: 726, SEQ ID NO: 727, SEQ ID NO: 728, SEQ ID NO: 729, SEQ ID NO: 730, SEQ ID NO: 731, SEQ ID NO: 732, SEQ ID NO: 733, SEQ ID NO: 734, SEQ ID NO: 735, SEQ ID NO: 736, SEQ ID NO: 737, SEQ ID NO: 738, SEQ ID NO: 739, SEQ ID NO: 740, SEQ ID NO: 741, SEQ ID NO: 742, SEQ ID NO: 743, SEQ ID NO: 744, SEQ ID NO: 745, SEQ ID NO: 746, SEQ ID NO: 747, SEQ ID NO: 748, SEQ ID NO: 749, SEQ ID NO: 750, SEQ ID NO: 751, SEQ ID NO: 752, SEQ ID NO: 753, SEQ ID NO: 754, SEQ ID NO: 755, SEQ ID NO: 756, SEQ ID NO: 757, SEQ ID NO: 758, SEQ ID NO: 759, SEQ ID NO: 760, SEQ ID NO: 761, SEQ ID NO: 762, SEQ ID NO: 763, SEQ ID NO: 764, SEQ ID NO: 765, SEQ ID NO: 766, SEQ ID NO: 767, SEQ ID NO: 768, SEQ ID NO: 769, SEQ ID NO: 770, SEQ ID NO: 771, SEQ ID NO: 772, SEQ ID NO: 773, SEQ ID NO: 774, SEQ ID NO: 775, SEQ ID NO: 776, SEQ ID NO: 777, SEQ ID NO: 778, SEQ ID NO: 779, SEQ ID NO: 780, SEQ ID NO: 781, SEQ ID NO: 782, SEQ ID NO: 783, SEQ ID NO: 784, SEQ ID NO: 785, SEQ ID NO: 786, SEQ ID NO: 787, SEQ ID NO: 788, SEQ ID NO: 789, SEQ ID NO: 790, SEQ ID NO: 791, SEQ ID NO: 792, SEQ ID NO: 793, SEQ ID NO: 794, SEQ ID NO: 795, SEQ ID NO: 796, SEQ ID NO: 797, SEQ ID NO: 798, SEQ ID NO: 799, SEQ ID NO: 800, SEQ ID NO: 801, SEQ ID NO: 802, SEQ ID NO: 803, SEQ ID NO: 804, SEQ ID NO: 805, SEQ ID NO: 806, SEQ ID NO: 807, SEQ ID NO: 808, SEQ ID NO: 809, SEQ ID NO: 810, SEQ ID NO: 811, SEQ ID NO: 812, SEQ ID NO: 813, SEQ ID NO: 814, SEQ ID NO: 815, SEQ ID NO: 816, SEQ ID NO: 817, SEQ ID NO: 818, SEQ ID NO: 819, SEQ ID NO: 820, SEQ ID NO: 821, SEQ ID NO: 822, SEQ ID NO: 823, SEQ ID NO: 824, SEQ ID NO: 825, SEQ ID NO: 826, SEQ ID NO: 827, SEQ ID NO: 828, SEQ ID NO: 829, SEQ ID NO: 830, SEQ ID NO: 831, SEQ ID NO: 832, SEQ ID NO: 833, SEQ ID NO: 834, SEQ ID NO: 835, SEQ ID NO: 836, SEQ ID NO: 837, SEQ ID NO: 838, SEQ ID NO: 839, SEQ ID NO: 840, SEQ ID NO: 841, SEQ ID NO: 842, SEQ ID NO: 843, SEQ ID NO: 844, SEQ ID NO: 845, SEQ ID NO: 846, SEQ ID NO: 847, SEQ ID NO: 848, SEQ ID NO: 849, SEQ ID NO: 850, SEQ ID NO: 851, SEQ ID NO: 852, SEQ ID NO: 853, SEQ ID NO: 854, SEQ ID NO: 855, SEQ ID NO: 856, SEQ ID NO: 857, SEQ ID NO: 858, SEQ ID NO: 859, SEQ ID NO: 860, SEQ ID NO: 861, SEQ ID NO: 862, SEQ ID NO: 863, SEQ ID NO: 864, SEQ ID NO: 865, SEQ ID NO: 866, SEQ ID NO: 867, SEQ ID NO: 868, SEQ ID NO: 869, SEQ ID NO: 870, SEQ ID NO: 871, SEQ ID NO: 872, SEQ ID NO: 873, SEQ ID NO: 874, SEQ ID NO: 875, SEQ ID NO: 876, SEQ ID NO: 877, SEQ ID NO: 878, SEQ ID NO: 879, SEQ ID NO: 880, SEQ ID NO: 881, SEQ ID NO: 882, SEQ ID NO: 883, SEQ ID NO: 884, SEQ ID NO: 885, SEQ ID NO: 886, SEQ ID NO: 887, SEQ ID NO: 888, SEQ ID NO: 889, SEQ ID NO: 890, SEQ ID NO: 891, SEQ ID NO: 892, SEQ ID NO: 893, SEQ ID NO: 894, SEQ ID NO: 895, SEQ ID NO: 896, SEQ ID NO: 897, SEQ ID NO: 898, SEQ ID NO: 899, SEQ ID NO: 900, SEQ ID NO: 901, SEQ ID NO: 902, SEQ ID NO: 903, SEQ ID NO: 904, SEQ ID NO: 905, SEQ ID NO: 906, SEQ ID NO: 907, SEQ ID NO: 908, SEQ ID NO: 909, SEQ ID NO: 910, SEQ ID NO: 911, SEQ ID NO: 912, SEQ ID NO: 913, SEQ ID NO: 914, SEQ ID NO: 915, SEQ ID NO: 916, SEQ ID NO: 917, SEQ ID NO: 918, SEQ ID NO: 919, SEQ ID NO: 920, SEQ ID NO: 921, SEQ ID NO: 922, SEQ ID NO: 923, SEQ ID NO: 924, SEQ ID NO: 925, SEQ ID NO: 926, SEQ ID NO: 927, SEQ ID NO: 928, SEQ ID NO: 929, SEQ ID NO: 930, SEQ ID NO: 931, SEQ ID NO: 932, SEQ ID NO: 933, SEQ ID NO: 934, SEQ ID NO: 935, SEQ ID NO: 936, SEQ ID NO: 937, SEQ ID NO: 938, SEQ ID NO: 939, SEQ ID NO: 940, SEQ ID NO: 941, SEQ ID NO: 942, SEQ ID NO: 943, SEQ ID NO: 944, SEQ ID NO: 945, SEQ ID NO: 946, SEQ ID NO: 947, SEQ ID NO: 948, SEQ ID NO: 949, SEQ ID NO: 950, SEQ ID NO: 951, SEQ ID NO: 952, SEQ ID NO: 953, SEQ ID NO: 954, SEQ ID NO: 955, SEQ ID NO: 956, SEQ ID NO: 957, SEQ ID NO: 958, SEQ ID NO: 959, SEQ ID NO: 960, or SEQ ID NOs: 3362-5698.

The recombinant DNA construct can include a nucleotide sequence that encodes (a) at least one copy of a CGI factor, (b) at least one copy each of two or more CGI factors, (c) at least one CGI factor precursor, (d) at least one CGI factor fragment, (e) at least one CGI factor motif, or (f) any combination of (a) to (e); any of these coding DNA sequences defines the corresponding encoded messenger RNA sequence. The heterologous promoter can be a bacterial promoter, a fungal promoter, an algal promoter, an animal promoter, or a plant promoter. The heterologous promoter can be a plant expressible promoter, i.e., a promoter that is functional for driving expression in a plant cell. In embodiments, the plant expressible promoter is selected from the group of promoters of a ubiquitin promoter, a cestrum yellow virus promoter, a corn TrpA promoter, a OsMADS 6 promoter, a maize H3 histone promoter, a corn sucrose synthetase 1 promoter, a corn alcohol dehydrogenase 1 promoter, a corn heat shock protein promoter, a maize mtl promoter, a pea small subunit RuBP carboxylase promoter, a rice actin promoter, a rice cyclophilin promoter, a Ti plasmid mannopine synthase promoter, a Ti plasmid nopaline synthase promoter, a Petunia chalcone isomerase promoter, a bean glycine rich protein 1 promoter, a potato patatin promoter, a lectin promoter, a CaMV 35S promoter, or a S-E9 small subunit RuBP carboxylase promoter. In some embodiments, the heterologous promoter is an inducible promoter, a tissue-specific promoter, a temporally specific promoter, or a developmentally specific promoter. Tissue-specific promoters are useful for limiting expression of the recombinant DNA construct and encoded CGI factor, CGI factor precursor, CGI factor fragment, or CGI factor motif to specific tissues (e.g., root, leaf, tuber, fruit, or seed) of a plant. In some embodiments, the heterologous promoter is a plant miRNA promoter, which can be inducible, tissue-specific, temporally specific, or developmentally specific; see, e.g., the tissue-specific promoters disclosed in U.S. Pat. No. 8,334,430 and the temporally specific promoters disclosed in U.S. Pat. No. 8,314,290. In some embodiments, the recombinant DNA construct includes further elements that are useful for expression control, such as expression-enhancing elements, transcript-stabilizing sequences, riboswitches, or recognition sites for miRNAs or siRNAs. For example, including a recognition site for a miRNA that is natively expressed in a specific tissue of a plant is expected to reduce or eliminate expression of the CGI factor, CGI factor precursor, CGI factor fragment, or CGI factor motif in that specific tissue. In additional embodiments, the recombinant DNA construct further includes a nucleotide sequence encoding at least one secretion signal peptide functional in a cell. In some embodiments, the vector includes a left T-DNA border and a right T-DNA border flanking the recombinant DNA construct. In some embodiments, the vector further comprises additional sequences flanking the recombinant DNA construct. The additional sequences can correspond to selectable markers, transposon ends, homologous arms, restriction sites, or other sequences suitable for downstream uses of the vector. In additional embodiments, the vector is a bacterial, viral, or viroid vector; when the vector is an RNA vector (e.g., an RNA virus or viroid vector), the vector can include RNA that encodes (a) at least one copy of a CGI factor, (b) at least one copy each of two or more CGI factors, (c) at least one CGI factor precursor, (d) at least one CGI factor fragment, (e) at least one CGI factor motif, or (f) any combination of (a) to (e).

### Transgenic Cells and Organisms

Transgenic cells of the present disclosure include the recombinant vectors or constructs described herein. In embodiments, the cell is selected from a bacterial cell, a fungal cell, an algal cell, an animal cell, or a plant cell. In some embodiments, the transgenic cell is a plant cell. In some embodiments, the plant cell is a dicot plant cell. In further embodiments, the dicot plant cell is selected from the group of a soybean cell, a sunflower cell, a tomato cell, a potato cell, a Brassica spp. cell, a cotton cell, a sugar beet cell, or a tobacco cell. In additional embodiments, the plant cell is a monocot plant cell. In further embodiments, the monocot plant cell is selected from the group of a barley cell, a maize cell, an oat cell, a rice cell, a sorghum cell, a sugar cane cell, or a wheat cell. In yet another embodiment, which can be combined with any of the preceding embodiments that has a transgenic cell, the CGI factor, the CGI factor precursor, the CGI factor fragment, or the CGI factor motif is (a) transiently expressed, or (b) stably expressed.

An additional aspect of the disclosure relates to a transgenic plant including the transgenic plant cell of any of the preceding embodiments. In some embodiments, the transgenic plant is chimeric, having some cells that are transgenic (e.g., expressing a recombinant DNA construct as disclosed herein) and some cells that are not transgenic. Related embodiments include a grafted plant, wherein the rootstock is transgenic (e.g., expressing a recombinant DNA construct as disclosed herein) and the grafted scion is not transgenic; or wherein the rootstock is not transgenic and the scion is transgenic. In embodiments, the modified genome is the nuclear genome of the plant; in other embodiments, the modified genome is the genome of the plant's chloroplasts or mitochondria. In some embodiments of this aspect, the transgenic plant is a dicot plant. In further embodiments of this aspect, the dicot plant is selected from the group of a soybean plant, a sunflower plant, a tomato plant, a Brassica spp. plant, a cotton plant, a sugar beet plant, or a tobacco plant. In additional embodiments of this aspect, the transgenic plant is a monocot plant. In further embodiments of this aspect, the monocot plant is selected from the group of a barley plant, a maize plant, an oat plant, a rice plant, a sorghum plant, a sugar cane plant, or a wheat plant. In yet another embodiment of this aspect, the plant has improved resistance to the fungal pathogen, in comparison to a control plant that does not include the transgenic plant cell. In still another embodiment of this aspect, the fungal pathogen is one of an Aspergillus species; Magnaporthe oryzae; Botrytis cinerea; a Puccinia species.; Fusarium graminearum; Fusarium oxysporum; Blumeria graminis; Mycosphaerella graminicola; a Colletotrichum species; Ustilago maydis; Melampsora lini, Phakopsora pachyrhizi, or Rhizoctonia solani. In another embodiment of this aspect, which can be combined with any of the preceding embodiments, the nucleotide sequence encoding the CGI factor, the CGI factor precursor, the CGI factor fragment, or the CGI factor motif does not occur in the genome of the fungal pathogen.

Plants and plant cells are of any species of interest, including dicots and monocots. Plants of interest include row crop plants, fruit-producing plants and trees, vegetables, trees, and ornamental plants including ornamental flowers, shrubs, trees, groundcovers, and turf grasses. Examples of commercially important cultivated crops, trees, and plants include: alfalfa (Medicago sativa), almonds (Prunus dulcis), apples (Malus x domestica), apricots (Prunus armeniaca, P. brigantine, P. mandshurica, P. mume, P. sibirica), artichoke (Cynara cardunculus var. scolymus), asparagus (Asparagus officinalis), avocado (Persea americana), bananas (Musa spp.), barley (Hordeum vulgare), beans (Phaseolus spp.), blueberries and cranberries (Vaccinium spp.), Brazil nut (Bertholletia excelsa), cacao (Theobroma cacao), calamansi (Citrus x microcarpa), canola and rapeseed or oilseed rape, (Brassica napus), Polish canola (Brassica rapa), and related cruciferous vegetables including broccoli, kale, cabbage, and turnips (Brassica carinata, B. juncea, B. oleracea, B. napus, B. nigra, and B. rapa, and hybrids of these), carnation (Dianthus caryophyllus), carrots (Daucus carota sativus), cashew (Anacardium occidentale), cassava (Manihot esculentum), celery (Apium graveolens), cherry (Prunus avium), chestnut (Castanea spp.), chickpea or garbanzo (Cicer arietinum), chicory (Cichorium intybus), chili peppers and other capsicum peppers (Capsicum annuum, C. frutescens, C. chinense, C. pubescens, C. baccatum), chrysanthemums (Chrysanthemum spp.), citron (Citrus medica), coconut (Cocos nucifera), coffee (wild and domesticated Coffea spp. including Coffea arabica, Coffea canephora, and Coffea liberica), cotton (Gossypium hirsutum L.), cowpea (Vigna unguiculata and other Vigna spp.), fava beans (Vicia faba), cucumber (Cucumis sativus), currants and gooseberries (Ribes spp.), date (Phoenix dactylifera), duckweeds (family Lemnoideae), eggplant or aubergine (Solanum melongena), elderberries (Sambucus spp.), eucalyptus (Eucalyptus spp.), flax (Linum usitatissumum L.), geraniums (Pelargonium spp.), ginger (Zingiber officinale), ginseng (Panax spp.), grapefruit (Citrus x paradisi), grapes (Vitis spp.) including wine grapes (Vitis vinifera and hybrids thereof), guava (Psidium guajava), hazelnut (Corylus avellana, Corylus spp.), hemp and cannabis (Cannabis sativa and Cannabis spp.), hops (Humulus lupulus), horseradish (Armoracia rusticana), irises (Iris spp.), jackfruit (Artocarpus heterophyllus), kiwifruits (Actinidia spp.), kumquat (Citrus japonica), lemon (Citrus limon), lentil (Lens culinaris), lettuce (Lactuca sativa), limes (Citrus spp.), lychee (Litchi chinensis), macadamias (Macadamia spp.), maize or corn (Zea mays L.), mandarin (Citrus reticulata), mango (Mangifera indica), mangosteen (Garcinia mangostana), melon (Cucumis melo), millets (Setaria spp., Echinochloa spp., Eleusine spp., Panicum spp., Pennisetum spp.), oats (Avena sativa), oil palm (Ellis quineensis), okra (Abelmoschus esculentus), olive (Olea europaea), onion (Allium cepa) and other alliums (Allium spp.), orange (Citrus sinensis), papaya (Carica papaya), parsnip (Pastinaca sativa), passionfruit (Passiflora edulis), pecan (Carya illinoinensis), peaches and nectarines (Prunus persica), pear (Pyrus spp.), pea (Pisum sativum), peanut (Arachis hypogaea), peonies (Paeonia spp.), persimmons (Diospyros kaki, Diospyros spp.), petunias (Petunia spp.), pineapple (Ananas comosus), pistachio (Pistacia vera), plantains (Musa spp.), plum (Prunus domestica), poinsettia (Euphorbia pulcherrima), pomelo (Citrus maxima), poplar (Populus spp.), potato (Solanum tuberosum), pumpkins and squashes (Cucurbita pepo, C. maxima, C. moschata), quince (Cydonia oblonga), raspberries (Rubus idaeus, Rubus occidentalis, Rubus spp.), rhubarbs (Rheum spp.), rice (Oryza sativa L.), roses (Rosa spp.), rubber (Hevea brasiliensis), rye (Secale cereale), safflower (Carthamus tinctorius L), satsuma (Citrus unshiu), sesame seed (Sesame indium), sorghum (Sorghum bicolor), sour orange (Citrus x aurantium), soursop (Annona muricata), soybean (Glycine max L.), strawberries (Fragaria spp., Fragaria x ananassa), sugar beet (Beta vulgaris), sugarcanes (Saccharum spp.), sunflower (Helianthus annuus), sweet potato (Ipomoea batatas), tamarind (Tamarindus indica), tangerine (Citrus tangerina), tea (Camellia sinensis), tobacco (Nicotiana tabacum L.), tomatillo (Physalis philadelphica), tomato (Solanum lycopersicum or Lycopersicon esculentum), tulips (Tulipa spp.), walnuts (Juglans spp. L.), watermelon (Citrulus lanatus), wheat (Triticum aestivum), and yams (Discorea spp.). Wild relatives of domesticated plants are also of interest.

A further aspect of the disclosure relates to a transgenic seed of the transgenic plant of any of the preceding embodiments, wherein said seed includes the recombinant DNA construct.

An additional aspect of the disclosure relates to an F1 progeny plant having as at least one parent the transgenic plant of any of the preceding embodiments, wherein the F1 progeny plant includes any of the recombinant DNA constructs of the preceding embodiments.

Yet another aspect of the disclosure relates to a harvested product produced from the transgenic plant of any of the preceding embodiments, wherein the harvested product includes the recombinant DNA construct. In some embodiments of this aspect, the harvested product is a fruit, a leaf, a stem, a flower, a root, a tuber, or a seed.

An additional aspect of the disclosure relates to a plant having a genome that is modified to express a heterologous DNA sequence that encodes a polypeptide including at least one conidial germination-inhibiting (CGI) factor, CGI factor precursor, CGI factor fragment, or CGI factor motif optionally fused to at least one plant secretion signal peptide, wherein the CGI factor, the CGI factor precursor, the CGI factor fragment, or the CGI factor motif inhibits conidial germination, growth, or reproduction of a fungal pathogen of the plant and wherein the CGI factor includes an amino acid sequence that has at least 80% sequence identity with a sequence selected from the group of SEQ ID NO: 961-1920, SEQ ID NO: 1957-2189, SEQ ID NO: 2194-2210, SEQ ID NO: 2215-2243, SEQ ID NO: 2457-3361, SEQ ID NO: 5707-5731, and SEQ ID NO: 5755-5911, or wherein the CGI factor motif includes at least one of SEQ ID NO: 1921-1956, and wherein the plant has improved resistance to the fungal pathogen, in comparison to a control plant that does not express the heterologous DNA sequence. The nucleotide sequence encoding the at least one CGI factor can include SEQ ID NO: 1-960 or 3362-5698. In additional embodiments of this aspect, the nucleotide sequence of the at least one CGI factor, CGI factor precursor, CGI factor fragment, or CGI factor motif does not occur in the genome of the fungal pathogen. In further embodiments of this aspect, which can be combined with any preceding embodiment, the fungal pathogen is one of an Aspergillus species; Magnaporthe oryzae; Botrytis cinerea; a Puccinia species.; Fusarium graminearum; Fusarium oxysporum; Blumeria graminis; Mycosphaerella graminicola; a Colletotrichum species; Ustilago maydis; Melampsora lini, Phakopsora pachyrhizi, or Rhizoctonia solani.

Yet another aspect of the disclosure relates to a plant including a cell containing a recombinant DNA construct for expressing a CGI factor, a CGI factor precursor, a CGI factor fragment, or a CGI factor motif and including: a heterologous promoter that is functional in the cell and is operably linked to a nucleic acid molecule including (a) a nucleotide sequence that encodes at least one conidial germination-inhibiting (CGI) factor, CGI factor precursor, CGI factor fragment, or CGI factor motif, and (b) a nucleotide sequence encoding at least one secretion signal peptide functional in the cells; wherein the CGI factor includes an amino acid sequence that has at least 80% sequence identity with a sequence selected from the group of SEQ ID NO: 961-1920, SEQ ID NO: 1957-2189, SEQ ID NO: 2194-2210, SEQ ID NO: 2215-2243, SEQ ID NO: 2457-3361, SEQ ID NO: 5707-5731, and SEQ ID NO: 5755-5911, or wherein the CGI factor motif includes at least one of SEQ ID NO: 1921-1956; and wherein the nucleotide sequence encoding the at least one CGI factor, CGI factor precursor, CGI factor fragment, or CGI factor motif optionally includes at least one codon optimized for expression in the cell. The nucleotide sequence encoding the at least one CGI factor can include SEQ ID NO: 1-960 or 3362-5698. In some embodiments of this aspect, the CGI factor, the CGI factor precursor, the CGI factor fragment, or the CGI factor motif is active and/or toxic. In further embodiments of this aspect, which can be combined with any preceding embodiment, the cell is a cell of the plant. In additional embodiments of this aspect, which can be combined with any preceding embodiment that has a cell that is not a cell of the plant, the cell is a bacterial or a fungal cell in or on the plant. In an embodiment, the cell is a bacterial or fungal cell heterologously expressing a CGI factor, a CGI factor precursor, or a CGI factor fragment, wherein a formulation comprising the cell, intact or lysed, is provided (e.g., by spraying, soaking, painting, or injecting) to an organism (e.g., a plant or seed) or object or environment to provide protection from fungal infection or growth. In some embodiments of this aspect, which can be combined with any preceding embodiment, the plant has improved resistance to the fungal pathogen, in comparison to a control plant that does not include the cell. In yet another embodiment of this aspect, the fungal pathogen is one of an Aspergillus species; Magnaporthe oryzae; Botrytis cinerea; a Puccinia species.; Fusarium graminearum; Fusarium oxysporum; Blumeria graminis; Mycosphaerella graminicola; a Colletotrichum species; Ustilago maydis; Melampsora lini, Phakopsora pachyrhizi, or Rhizoctonia solani. In still another embodiment of this aspect, which can be combined with any preceding embodiment, the nucleotide sequence encoding the at least one CGI factor, CGI factor precursor, CGI factor fragment, or CGI factor motif does not occur in the genome of the fungal pathogen.

### Fungicides

Fungicides that Interfere with Fungal Cell Walls or Fungal Cell Membranes

In some embodiments, the fungicide interferes with fungal cell walls or fungal cell membranes. In certain embodiments, the fungicide comprises azoles. In certain embodiments, the fungicide comprises at least one fungicide that is classified as a triazole and/or FRAC group 3 fungicide as defined in the FRAC Code List 2022, or a combination of the foregoing fungicides.

In some embodiments, the fungicide comprises inhibitors of fungal lipid synthesis or transport or membrane integrity or function. In certain embodiments, the fungicide is a polyene macrolide. In certain embodiments, the fungicide comprises at least one fungicide that is classified as a FRAC group 48 fungicide as defined in the FRAC Code List 2022.

In some embodiments, the fungicide that interferes with fungal cell wall or fungal cell membranes interacts with and disrupts fungal cell membranes. In certain embodiments, the fungicide comprises at least one fungal cell membrane-interacting antifungal peptide (AMP).

In some embodiments, the fungicide inhibits beta-glucan synthesis in the fungal cell wall. In certain embodiments, the fungicide comprises at least one cyclic lipopeptide. In some embodiments, a combination of any the foregoing fungicides can be used in the methods herein.

### Azoles and Similar Heterocycles

The global Fungicide Resistance Action Committee (FRAC) has designated “Group 3 Fungicides” (also referred to as “FRAC Code 3” or “FRAC 3”) as those having a membrane sterol biosynthesis-based mode of action (MOA) targeting C14-demethylase in sterol biosynthesis (e.g., erg11, cyp51). These fungicides are also referred to as “SBI Class I” fungicides and demethylation (DMI) inhibitors. In some embodiments, the FRAC 3 fungicide is a nitrogen-containing heterocycle having a 5- to 6-membered ring containing 1-3 nitrogen atoms. In embodiments, the FRAC 3 fungicide is an azole.

In some embodiments, the fungicide that interferes with fungal cell walls or fungal cell membranes has a mode of action that involves inhibition of sterol biosynthesis in fungal cell membranes by targeting C14-demethylase in sterol biosynthesis. In some embodiments, the fungicide inhibits ergosterol biosynthesis. In some embodiments, the fungicide is classified as a FRAC group 3 fungicide.

In certain embodiments, the fungicide includes piperazines, pyridines, pyrimidines, imidazoles, triazoles, triazolinthiones, or any combinations of the foregoing.

In certain embodiments, the fungicide includes triforine, pyrifenox pyrisoxazole, fenarimol, nuarimol, imazalil, oxpoconazole, pefurazoate, prochloraz, triflumizole, azaconazole, bitertanol, bromuconazole, cyproconazole, difenoconazole, diniconazole, epoxiconazole, etaconazole, fenbuconazole, fluquinconazole, flusilazole, flutriafol, hexaconazole, imibenconazole, ipconazole, mefentrifluconazole, fluconazole, metconazole, myclobutanil, penconazole, propiconazole, simeconazole, tebuconazole, tetraconazole, triadimefon, triadimenol, triticonazole, prothioconazole, or any combinations of the foregoing.

In some embodiments, the fungicide is a triazole. In some embodiments, the fungicide is metconazole. In some embodiments, the fungicide is fluconazole.

It should generally be understood that any combination of small heterocyclic compounds classified as FRAC 3 fungicides can be used in the methods described herein. For example, in some embodiments, a plurality of triazole fungicides can be used. In other embodiments, a metconazole fungicide is used. In other embodiments, a fluconazole fungicide is used. In yet other embodiments, a combination of metconazole and fluconazole is used.

### Polyene Macrolides

The global Fungicide Resistance Action Committee (FRAC) has designated “Group 48 Fungicides” (also referred to as “FRAC Code 48” or “FRAC 48”) as those having a lipid synthesis or transport or membrane integrity or function-based mode of action (MOA) targeting ergosterol binding. These fungicides are also referred to as polyenes or polyene macrolides.

In some embodiments, the fungicide is a polyene macrolide fungicide. In some embodiments, the polyene macrolide fungicide has a mode of action that involves targeting ergosterol in a fungus, e.g., binding to ergosterol in the cell membrane of a fungus.

In certain embodiments, the polyene macrolide fungicide includes an amphoteric macrolide. In certain embodiments, the polyene macrolide fungicide is isolated from a Streptomyces sp., e.g., from Streptomyces natalensis, Streptomyces chattanoogensis, or Streptomyces nodosus.

In certain embodiments, the polyene macrolide fungicide includes natamycin (pimaricin), amphotericin, nystatin, or any combinations of the foregoing.

In some embodiments, the polyene macrolide fungicide is natamycin.

### AMPs

In some embodiments, the fungicide that interferes with fungal cell wall or fungal cell membranes has a mode of action that involves at least one antifungal peptide that interacts with and disrupts the fungal cell membrane and/or the fungal cell wall. Fungicides that interfere with fungal cell wall or fungal cell membranes can inhibit fungal spore germination, delay growth of fungal hyphae, and/or lead to partial lysis of fungal hyphae.

In certain embodiments, the antifungal peptide includes cell membrane-interacting antifungal peptides and/or antimicrobial peptides (AMPs).

In certain embodiments, the antifungal peptide includes at least one of the group consisting of drosomycin, cecropin, diptericin, drosocin, metchnikowin, attacin, nisin, thanatin, proline-rich peptides, glycine-rich peptides, and any combinations of the foregoing.

In some embodiments, the antifungal peptide is drosomycin.

### Cyclic Lipopeptides

In some embodiments, the fungicide is a cyclic lipopeptide. In some embodiments, the cyclic lipopeptide has a mode of action that involves inhibition of beta-glucan synthesis in the fungal cell wall.

In certain embodiments, the cyclic lipopeptide fungicide includes echinocandins. Echinocandins are cyclic hexapeptides that are N-linked to a fatty acyl side chain. Echinocandins inhibit β-1,3-glucan synthase, thus interfering with cell wall biosynthesis.

In certain embodiments, the cyclic lipopeptide fungicide includes caspofungin, micafungin, anidulafungin, or any combinations of the foregoing.

In some embodiments, the cyclic lipopeptide fungicide is an echinocandin. In some embodiments, the cyclic lipopeptide fungicide is caspofungin.

It should be understood that, in some embodiments of the foregoing, any combinations of the fungicides described herein can be employed. For example, in certain embodiments, a combination of one or more fungicides from the azoles, polyene macrolides, AMPs, or cyclic lipopeptides can be used.

In some embodiments, the fungus is contacted simultaneously with an effective amount of at least one conidial germination-inhibiting (CGI) factor, CGI factor precursor, CGI factor fragment, or CGI factor motif and an effective amount of at least one fungicide described herein. In other embodiments, the fungus is contacted sequentially (in any order) with an effective amount of at least one CGI factor, CGI factor precursor, CGI factor fragment, or CGI factor motif and an effective amount of at least one fungicide as described herein. In some embodiments, the antifungal compositions provided herein include at least one CGI factor, CGI factor precursor, CGI factor fragment, or CGI factor motif and at least one fungicide described herein.

### Fungal Signal Transduction-Inhibiting Fungicides

In some embodiments, the fungal signal transduction-inhibiting fungicide comprises inhibitors of fungal signal transduction. In certain embodiments, the fungicide is classified as a FRAC group 12 fungicide as defined in the FRAC Code List 2022.

### FRAC 12

The global Fungicide Resistance Action Committee (FRAC) has designated “Group 12 Fungicides” (also referred to as “FRAC Code 12” or “FRAC 12”) as those having a signal transduction-based mode of action (MOA) targeting MAP/Histidine-Kinase in osmotic signal transduction (e.g., os-2, HOG1). These fungicides are also referred to as phenylpyrrole fungicides or PP-fungicides, as phenylpyrroles are a member of this group.

In some embodiments, the phenylpyrrole fungicide has a mode of action that involves targeting signal transduction in a fungus, e.g., targeting MAP/Histidine-Kinase in osmotic signal transduction in a fungus. In some embodiments, the phenylpyrrole fungicide is fludioxonil or fenpiclonil, or a combination thereof.

In certain embodiments, FRAC group 12 fungicides include phenylpyrroles. In certain embodiments, FRAC group 12 fungicides include fenpiclonil, fludioxonil, or a combination thereof. In some embodiments, the fungicide is fludioxonil. In some embodiments, the fungicide is fenpiclonil.

It should generally be understood that, in some embodiments of the foregoing, any combination of the fungicides herein can be used in the methods described herein. For example, in some embodiments, a plurality of FRAC group 12 fungicides can be used.

### Fungal Respiration-Inhibiting Fungicides

In some embodiments, the fungicide is a fungal respiration-inhibiting fungicide. In some embodiments, the fungicide comprises inhibitors of fungal respiration. In certain embodiments, the fungicide is classified as a FRAC group 7 fungicide or a FRAC group 11 fungicide as defined in the FRAC Code List 2022, or a combination of the foregoing fungicides.

### FRAC 7

The global Fungicide Resistance Action Committee (FRAC) has designated “Group 7 Fungicides” (also referred to as “FRAC Code 7” or “FRAC 7”) as those having a respiration-based mode of action (MOA) targeting respiratory complex II: succinate-dehydrogenase (SDH; also known as succinate-coenzyme Q reductase (SQR)). Thus, these fungicides are also referred to as succinate-dehydrogenase inhibitors (SDHI).

In some embodiments, the fungal respiration-inhibiting fungicide is a succinate dehydrogenase inhibitor (SDHI) that inhibits succinate dehydrogenase (SDH), and is classified as a FRAC group 7 fungicide. In some embodiments, the FRAC group 7 fungicide is an amide, such as a benzamide, a pyridine amide, or a carboxamide.

In certain embodiments, FRAC group 7 fungicides include phenyl-benzamides, phenyl-oxo-ethyl thiophene amide, pyridinyl-ethyl-benzamides, phenyl-cyclobutyl-pyridineamide, furan-carboxamides, oxathiin-carboxamides, thiazole-carboxamides, pyrazole-4-carboxamides, N-cyclopropyl-N-benzyl-pyrazole-carboxamides, N-methoxy-(phenyl-ethyl)-pyrazole-carboxamides, pyridine-carboxamides, and pyrazine-carboxamides, or any combinations of the foregoing.

In certain embodiments, FRAC group 7 fungicides include benodanil, flutolanil, mepronil, isofetamid, fluopyram, cyclobutrifluram, fenfuram, carboxin, oxycarboxin, thifluzamide, benzovindiflupyr, bixafen, fluindapyr, fluxapyroxad, furametpyr, inpyrfluxam, isopyrazam, penflufen, penthiopyrad, sedaxane, isoflucypram, pydiflumetofen, boscalid, and pyraziflumid, or any combinations of the foregoing.

In some embodiments, the FRAC group 7 fungicide is a pyridine-carboxamide. In one embodiment, the FRAC group 7 fungicide is boscalid.

### FRAC 11

The global Fungicide Resistance Action Committee (FRAC) has designated “Group 11 Fungicides” (also referred to as “FRAC Code 11” or “FRAC 11”) as those having a respiration-based mode of action (MOA) targeting respiratory complex III:cytochrome bc1 (ubiquinol oxidase) at Qo site (cyt b gene). Thus, these fungicides are also referred to as quinone outside inhibitor (QoI) fungicides.

In some embodiments, the fungal respiration-inhibiting fungicide acts as a quinone outside inhibitor that inhibits respiratory chain at the coenzyme Q-cytochrome c reductase step. In certain embodiments, the fungal respiration-inhibiting fungicide is a quinone outside inhibitor (QoI) that inhibits respiration by targeting cytochrome bc1 (ubiquinol oxidase) at the Qo site, and is classified as a FRAC group 11 fungicide.

In certain embodiments, FRAC group 11 fungicides include methoxy-acrylates, methoxy-acetamide, methoxy-carbamates, oximino-acetates, oximino-acetamides, oxazolidine-diones, dihydro-dioxazines, imidazolinones, and benzyl-carbamates, synthetic strobilurin analogues, or any combinations of the foregoing.

In some embodiments, the FRAC group 11 fungicides are FRAC group 11A fungicides, which include, for example, tetrazolinones. In one embodiment, the FRAC group 11A fungicide is metyltetraprole.

In certain embodiments, FRAC group 11 fungicides include azoxystrobin, coumoxystrobin, enoxastrobin, flufenoxystrobin, picoxystrobin, pyraoxystrobin, mandestrobin, pyraclostrobin, pyrametostrobin, triclopyricarb, kresoxim-methyl, trifloxystrobin, dimoxystrobin, fenaminstrobin, metominostrobin, orysastrobin, famoxadone, fluoxastrobin, fenamidone, and pyribencarb, and pyraziflumid, or any combinations of the foregoing.

In certain embodiments, the FRAC group 11 fungicide is an oximino-acetate. In one embodiment, the FRAC group 11 fungicide is trifloxystrobin.

It should generally be understood that, in some embodiments of the foregoing, any combination of the FRAC 7 and FRAC 11 fungicides can be used in the methods described herein. For example, in some embodiments, a plurality of FRAC group 7 fungicides can be used. In other embodiments, a plurality of FRAC group 11 fungicides can be used. In yet other embodiments, a combination of FRAC group 7 fungicide(s) and FRAC group 11 fungicide(s) can be used.

Fungal Amino Acid and/or Protein Synthesis-Inhibiting Fungicides

In some embodiments, the fungicide is a fungal amino acid and/or protein synthesis-inhibiting fungicide. In certain embodiments, the fungicide comprises inhibitors of fungal methionine biosynthesis. In certain embodiments, the fungicide is classified as a FRAC group 9 fungicide as defined in the FRAC Code List 2022.

### FRAC9

The global Fungicide Resistance Action Committee (FRAC) has designated “Group 9 Fungicides” (also referred to as “FRAC Code 9” or “FRAC 9”) as those having an amino acid and/or protein synthesis-based mode of action (MOA) targeting methionine biosynthesis. These fungicides are also referred to as anilino-pyrimidine fungicides or AP-fungicides.

In certain embodiments, FRAC group 9 fungicides include anilino-pyrimidines.

In certain embodiments, the fungicide is cyprodinil, mepanipyrim, pyrimethanil, or any combinations of the foregoing. In some embodiments, the fungicide is cyprodinil.

It should be understood that, in some embodiments of the foregoing, any combination of the fungicides described herein can be employed in the methods herein. For example, in certain embodiments, a plurality of FRAC group 9 fungicides can be used.

In some embodiments, the fungus is contacted simultaneously with an effective amount of at least one conidial germination-inhibiting (CGI) factor and an effective amount of at least one fungicide described herein. In other embodiments, the fungus is contacted sequentially (in any order) with an effective amount of at least one conidial germination-inhibiting (CGI) factor and an effective amount of at least one fungicide as described herein. In some embodiments, the antifungal compositions provided herein include at least one CGI factor and at least one fungicide described herein.

### Compositions

Yet another aspect of the disclosure relates to an antifungal or fungicidal composition including at least one CGI factor, the CGI factor precursor, the CGI factor fragment, or the CGI factor motif of any one of the preceding embodiments, and at least one fungicide of any one of the preceding embodiments. In some embodiments, the fungicide is classified as a FRAC group 3 fungicide, a polyene macrolide fungicide, a cell membrane-interacting antifungal peptides and/or antimicrobial peptide (AMP), or a cyclic lipopeptide fungicide. In some embodiments, fungicide inhibits sterol biosynthesis in fungal cell membranes, and is classified as a FRAC group 3 fungicide. In some embodiments, the FRAC group 3 fungicide is an azole or a nitrogen-containing heterocycle having a 5- to 6-membered ring containing 1-3 nitrogen atoms. In some embodiments, the FRAC group 3 fungicide is a piperazine, a pyridine, a pyrimidine, an imidazole, a triazole, a triazolinthione, or any combination thereof. In some embodiments, the FRAC group 3 fungicide is triforine, pyrifenox pyrisoxazole, fenarimol, nuarimol, imazalil, oxpoconazole, pefurazoate, prochloraz, triflumizole, azaconazole, bitertanol, bromuconazole, cyproconazole, difenoconazole, diniconazole, epoxiconazole, etaconazole, fenbuconazole, fluquinconazole, flusilazole, flutriafol, hexaconazole, imibenconazole, ipconazole, mefentrifluconazole, fluconazole, metconazole, myclobutanil, penconazole, propiconazole, simeconazole, tebuconazole, tetraconazole, triadimefon, triadimenol, triticonazole, prothioconazole, or any combination thereof. In some embodiments, the FRAC 3 group fungicide is a triazole, a metconazole, or a fluconazole. In some embodiments, the fungicide is a polyene macrolide fungicide. In some embodiments, the polyene macrolide fungicide targets ergosterol in a fungus, is an amphoteric macrolide, and/or is isolated from a Streptomyces species. In some embodiments, the polyene macrolide fungicide is natamycin (pimaricin), amphotericin, nystatin, or any combination thereof. In some embodiments, the polyene macrolide fungicide is natamycin. In some embodiments, the fungicide is an antifungal peptide that interacts with and disrupts the fungal cell membrane and/or the fungal cell wall. In some embodiments, the antifungal peptide is a cell membrane-interacting antifungal peptide and/or antimicrobial peptide (AMP). In some embodiments, the antifungal peptide is drosomycin, cecropin, diptericin, drosocin, metchnikowin, attacin, nisin, thanatin, a proline-rich peptide, a glycine-rich peptide, or any combination thereof. In some embodiments, the antifungal peptide is drosomycin. In some embodiments, the fungicide is a cyclic lipopeptide. In some embodiments, the cyclic lipopeptide inhibits beta-glucan synthesis in the fungal cell wall. In some embodiments, the cyclic lipopeptide is an echinocandin. In some embodiments, the cyclic lipopeptide is caspofungin, micafungin, anidulafungin, or any combination thereof. In some embodiments, the cyclic lipopeptide is caspofungin. In some embodiments, the fungicide is classified as a FRAC group 12 fungicide. In some embodiments, the fungicide is a phenylpyrrole fungicide with a mode of action that involves targeting signal transduction in a fungus. In some embodiments, the phenylpyrrole fungicide is fludioxonil or fenpiclonil, or a combination thereof. In some embodiments, the phenylpyrrole fungicide is fludioxonil or fenpiclonil. In some embodiments, the fungicide is classified as a FRAC group 7 fungicide or a FRAC group 11 fungicide. In some embodiments, the fungicide is a succinate dehydrogenase inhibitor (SDHI) that inhibits succinate dehydrogenase (SDH), and is classified as a FRAC group 7 fungicide. In some embodiments, the FRAC group 7 fungicide is an amide. In some embodiments, the FRAC group 7 fungicide is a benzamide, a pyridine amide, or a carboxamide. In some embodiments, the FRAC group 7 fungicide is a phenyl-benzamide, phenyl-oxo-ethyl thiophene amide, pyridinyl-ethyl-benzamide, phenyl-cyclobutyl-pyridineamide, furan-carboxamide, oxathiin-carboxamide, thiazole-carboxamide, pyrazole-4-carboxamide, N-cyclopropyl-N-benzyl-pyrazole-carboxamide, N-methoxy-(phenyl-ethyl)-pyrazole-carboxamide, pyridine-carboxamide, or pyrazine-carboxamide, or any combination thereof. In some embodiments, the FRAC group 7 fungicide is benodanil, flutolanil, mepronil, isofetamid, fluopyram, cyclobutrifluram, fenfuram, carboxin, oxycarboxin, thifluzamide, benzovindiflupyr, bixafen, fluindapyr, fluxapyroxad, furametpyr, inpyrfluxam, isopyrazam, penflufen, penthiopyrad, sedaxane, isoflucypram, pydiflumetofen, boscalid, or pyraziflumid, or any combination thereof. In some embodiments, the FRAC group 7 fungicide is a pyridine-carboxamide. In some embodiments, the FRAC group 7 fungicide is boscalid. In some embodiments, the fungicide is a quinone outside inhibitor (QoI) that inhibits respiration by targeting cytochrome bc1 (ubiquinol oxidase) at the Qo site, and is classified as a FRAC group 11 fungicide. In some embodiments, the FRAC group 11 fungicide is a synthetic strobilurin analogue. In some embodiments, the FRAC group 11 fungicide is a FRAC group 11A fungicide. In some embodiments, the FRAC group 11 fungicide is a methoxy-acrylate, methoxy-acetamide, methoxy-carbamate, oximino-acetate, oximino-acetamide, oxazolidine-dione, dihydro-dioxazine, imidazolinone, benzyl-carbamate, or synthetic strobilurin analogue, or any combination thereof. In some embodiments, the FRAC group 11 fungicide is azoxystrobin, coumoxystrobin, enoxastrobin, flufenoxystrobin, picoxystrobin, pyraoxystrobin, mandestrobin, pyraclostrobin, pyrametostrobin, triclopyricarb, kresoxim-methyl, trifloxystrobin, dimoxystrobin, fenaminstrobin, metominostrobin, orysastrobin, famoxadone, fluoxastrobin, fenamidone, pyribencarb, or pyraziflumid, or any combination thereof. In some embodiments, the FRAC group 11 fungicide is an oximino-acetate. In some embodiments, the FRAC group 11 fungicide is trifloxystrobin. In some embodiments, the fungicide is classified as a FRAC group 9 fungicide. In some embodiments, the fungicide is an anilino-pyrimidine fungicide that targets methionine biosynthesis, and is classified as a FRAC group 9 fungicide. In some embodiments, the FRAC 9 group fungicide is cyprodinil, mepanipyrim, pyrimethanil, or any combination thereof. In some embodiments, the FRAC 9 group fungicide is cyprodinil.

In some embodiments of this aspect, the composition is formulated as one of a seed treatment, a foliar spray treatment, a foliar drench treatment, an injectable formulation, a Ready-To-Use (RTU) formulation, a produce coating, a suspension concentrate, a tank-mix, an aerosol, a root dip, a drench, a fog, a soil treatment, an irrigation formulation, or a sprinkler formulation. In further embodiments of this aspect, the agriculturally acceptable carrier includes a solid carrier, a liquid carrier, a gel carrier, a suspension, or an emulsion. In additional embodiments of this aspect, the agriculturally acceptable carrier includes one or more of an adjuvant, an inert component, a dispersant, a surfactant, a tackifier, a binder, or a stabilizer. Adjuvants and other components useful in agricultural formulations are described, e.g., in the Compendium of Herbicidal Adjuvants, 13^{th} edition, 2016; available at siu-weeds[dot]com/adjuvants/index-adj[dot]html. In some embodiments of this aspect, such agricultural formulations further include one or more additional components, such as an herbicide, insecticide, nematicide, fungicide (other than the CGI factor, CGI factor precursor, CGI factor fragment, or CGI factor motifs and fungicides herein disclosed), attractant, or bait. In some embodiments of this aspect, the composition is formulated for application to human-built structures (e.g., buildings, fencing, walls) or artifacts (e.g., furniture, clothing, fabrics) or for incorporation in materials useful for making human-built structures or artifacts. In some embodiments of this aspect, the composition is incorporated as an addition to food or feed, e.g., products processed from plants. In some embodiments of this aspect, the compositions are formulated for and applied to plants or plant parts (e.g., fruit, seed, grain, flowers, leaves, stems, tubers, roots, or bulbs) prior to harvest or to the harvested plants or plant parts, to prevent fungal growth and/or to improve storage or shelf-life of the harvested plants or plant parts. In additional embodiments of this aspect, the compositions are formulated as slow-release or controlled-release formulations.

An additional aspect of the disclosure relates to an antifungal composition including an effective amount of at least one conidial germination-inhibiting (CGI) factor, CGI factor precursor, CGI factor fragment, or CGI factor motif, and that includes (a) an amino acid sequence of a CGI factor that has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with a sequence selected from the group SEQ ID NO: 961-1920, SEQ ID NO: 1957-2189, SEQ ID NO: 2194-2210, SEQ ID NO: 2215-2243, SEQ ID NO: 2457-3361, SEQ ID NO: 5707-5731, and SEQ ID NO: 5755-5911; or (b) an amino acid sequence of a CGI factor motif; a fungicide that interferes with fungal cell wall or fungal cell membranes; and a carrier. In some embodiments of this aspect, the CGI factor motif includes at least one of SEQ ID NO: 1921-1956. In additional embodiments of this aspect, which can be combined with any preceding embodiment, the CGI factor, CGI factor precursor, CGI factor fragment, or CGI factor motif is active and/or toxic. In further embodiments of this aspect, which can be combined with any preceding embodiment, the carrier is selected from an agriculturally acceptable carrier, or a pharmaceutically acceptable carrier. In additional embodiments of this aspect, which can be combined with any preceding embodiment, the composition is formulated as a liquid, a gel, an emulsion, a suspension, an encapsulation, a solid, a powder, a coating, a spray, a soil drench, granules, a seed coat, or a bait. In some embodiments, the composition is encapsulated by or formulated with a nanocarrier, e.g., polymer, clay, or zein micro- or nanoparticles, or lipid micro- or nanoparticles; see, e.g., Wang et al. (2022) Nature Nanotechnol., 17:347-360, DOI: 10.1038/s41565-022-01082-8, incorporated by reference herein. In some embodiments of this aspect, such agricultural formulations further include one or more additional components, such as an herbicide, insecticide, nematicide, fungicide (other than the CGI factor, CGI factor precursor, CGI factor fragment, or CGI factor motifs and fungicides herein disclosed), attractant, or bait. In some embodiments of this aspect, the composition is formulated for application to human-built structures (e.g., buildings, fencing, walls) or artifacts (e.g., furniture, clothing, fabrics) or for incorporation in materials useful for making human-built structures or artifacts. In some embodiments of this aspect, the composition is incorporated as an addition to food or feed, e.g., products processed from plants. In additional embodiments of this aspect, the compositions are formulated as slow-release or controlled-release formulations.

Pharmaceutically acceptable carriers and excipients in the present compositions are nontoxic to recipients at the dosages and concentrations employed. Acceptable carriers and excipients can include buffers such as phosphate, citrate, HEPES, and TAE, antioxidants such as ascorbic acid and methionine, preservatives such as hexamethonium chloride, octadecyldimethylbenzyl ammonium chloride, resorcinol, and benzalkonium chloride, proteins such as human serum albumin, gelatin, and immunoglobulins, hydrophilic polymers such as dextran and polyvinylpyrrolidone, amino acids such as glycine, glutamine, histidine, and lysine, and carbohydrates such as glucose, mannose, sucrose, and sorbitol. The compositions can be formulated according to conventional pharmaceutical practice. In embodiments, the composition is formulated as a pharmaceutical (or veterinary) formulation such as, but not limited to, a liquid, emulsion, reverse emulsion, suspension, gel, cream, ointment, injectable, or solid, or any appropriate formulation, e.g., for topical, oral, intravenous, intramuscular, intraperitoneal, aerosolized, or nebulized administration, or for application to a subject by a device, such as a transdermal patch, bandage, tape, film, coating, or solid or porous matrix or surface. The concentration of the compound in the formulation will vary depending upon a number of factors, including the dosage of the active agent to be administered, and the route of administration.

Additional embodiments include formulations designed for agricultural, pharmaceutical, or veterinary use, wherein the CGI factor (or precursor thereof) is provided in a cell (viable and intact, or non-viable and intact) or as a cell-derived preparation, such as lyophilized cells, a suspension or pellet of lysed cells, or a cell membrane-bound particle or analogous synthetic lipid mono- or bilayer-bound particle (e.g., a minicell, exosome, or vesicle; see, e.g., exosomes and vesicles described in Di Gioia et al. (2020) Open Medicine, 15:1096-1122, Mozafari (2010) “Nanoliposomes: Preparation and Analysis”, Chapter 2 in “Liposomes, Methods in Molecular Biology, volume 605”, Weissig (Ed.), DOI 10.1007/978-1-60327-360-2_2, and International Patent Application Publication WO2019/222379; also see, e.g., non-replicating minicells described in Nguyen (2011), “Cell Division Gene from Bacteria in Minicell Production for Therapy, Advances in Cancer Therapy, Gali-Muhtasib (Ed.), ISBN: 9789533077031, available at www[dot]intechopen[dot]com/books/advances-in-cancer-therapy/cell-division-gene-from-bacteria-in-minicell-production-for-therapy; Ni et al. (2021), ACS Synth. Biol., 10_1284-1291, and International Patent Application Publication WO2020/123569; all references cited in this paragraph are incorporated by reference in their entirety. Examples of such cell-derived formulations include fermentation preparations of bacterial, fungal, plant, or animal (e.g., insect) cells or minicells expressing one or more CGI factors and grown in culture; formulations made from such fermentation preparations can be provided (e.g., by spraying, soaking, painting, or injecting) to a subject organism or object or environment to provide protection from fungal infection or growth.

Still another aspect of the disclosure relates to a composition having antifungal properties, including a substrate or matrix that is complexed with at least one conidial germination-inhibiting (CGI) factor, CGI factor precursor, CGI factor fragment, or CGI factor motif that is active and/or toxic, wherein the CGI factor includes an amino acid sequence that has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with a sequence selected from the group of SEQ ID NO: 961-1920, SEQ ID NO: 1957-2189, SEQ ID NO: 2194-2210, SEQ ID NO: 2215-2243, SEQ ID NO: 2457-3361, SEQ ID NO: 5707-5731, and SEQ ID NO: 5755-5911, or wherein the CGI factor motif includes at least one of SEQ ID NO: 1921-1956 and a fungicide that interferes with fungal cell wall or fungal cell membranes. In some embodiments of this aspect, the CGI factor, the CGI factor precursor, the CGI factor fragment, or the CGI factor motif is active and/or toxic. In further embodiments of this aspect, which can be combined with any preceding embodiment, the complexation between the substrate or matrix with the at least one CGI factor, CGI factor precursor, CGI factor fragment, or CGI factor motif is through: (a) covalent bonding, (b) non-covalent bonding, or (c) a combination of (a) and (b). In some embodiments of this aspect, the substrate or matrix includes polypeptides. In additional embodiments of this aspect, which can be combined with any preceding embodiment that has a substrate or matrix, the substrate or matrix includes self-assembling peptides. In some embodiments, a substrate or matrix is complexed with (a) at least one conidial germination-inhibiting (CGI) factor, CGI factor precursor, CGI factor fragment, or CGI factor motif, and (b) at least one fungicide that interferes with fungal cell wall or fungal cell membranes, fungal signal transduction-inhibiting fungicide, fungal respiration-inhibiting fungicide, or fungal amino acid and/or protein synthesis-inhibiting fungicide. In some embodiments, the fungicide that interferes with fungal cell wall or fungal cell membranes is applied to a substrate or matrix that is complexed with the CGI factor, CGI factor precursor, CGI factor fragment, or CGI factor motif.

Any suitable substrate or matrix known to those in the art can be applied to the present disclosure. In some embodiments, the substrate or matrix comprises polypeptides. In some embodiments, the polypeptides are self-assembling peptides. In some embodiments, the self-assembling peptide is (M)(YEYK)ₙYEY (SEQ ID NO: 2194), where n=3 or n is between 3-10, and where methionine is the terminal and optional amino acid, is covalently or non-covalently linked to one or more CGI peptides. Self-assembling peptides have been known to those of ordinary skill in the art, as demonstrated by Miki et al. (2021) Nature Communications, 21:3412, DOI: 10.1038/s41467-021-23794-6, which is specifically and entirely incorporated by reference herein for everything it teaches. In some embodiments, the complexation between the substrate or matrix and at least one CGI peptide is through covalent bonding. In some embodiments, the complexation between the substrate or matrix and at least one CGI peptide is through non-covalent bonding. In some embodiments, the complexation between the substrate or matrix and at least one CGI peptide is through a combination of covalent bonding and non-covalent bonding.

### Methods of Providing Fungal Resistance

A further aspect of the disclosure related to methods of providing an organism with resistance to a fungal pathogen of the organism, including contacting the organism with an antifungal composition of the present disclosure, whereby the organism becomes resistant to the fungal pathogen. In some embodiments, the organism is contacted with both the at least one CGI factor, CGI factor precursor, CGI factor fragment, or CGI factor motif and the at least one fungicide at the same time, or the organism is contacted with the at least one CGI factor, CGI factor precursor, CGI factor fragment, or CGI factor motif and the at least one fungicide sequentially. In some embodiments, the organism is pre-treated with either the at least one CGI factor, CGI factor precursor, CGI factor fragment, or CGI factor motif or the at least one fungicide, and the organism is contacted with the other at a later time.

In some aspects, the disclosure related to methods of providing an organism with resistance to a fungal pathogen of the organism, including expressing in a cell of the organism the recombinant DNA construct of any one of the preceding embodiments, wherein the heterologous promoter is functional in the organism, and then contacting the organism with at least one fungicide of the present disclosure. In further embodiments of this aspect, the nucleotide sequence encoding the CGI factor, the CGI factor precursor, the CGI factor fragment, or the CGI factor motif does not occur in the genome of the fungal pathogen. In additional embodiments of this aspect, the recombinant DNA construct is introduced into the cell of the organism by (a) transfection; (b) by inheritance from a parent cell; or (c) by fusion with a donor cell including the recombinant DNA construct. In yet further embodiments of this aspect, the organism is a plant, and wherein the recombinant DNA construct is provided to the plant by (a) transformation, or (b) inheritance from at least one parent plant that contained the recombinant DNA construct. Transformation can be stable or transient. In still another embodiment of this aspect, the plant is selected from the group of a maize plant, a soybean plant, a wheat plant, a rice plant, a cotton plant, a potato plant, a tomato plant, a Brassica spp. plant, or a sugar beet plant.

An additional aspect of the disclosure relates to methods of providing an organism with resistance to a fungal pathogen of the organism, including contacting the organism with the vector of any of the preceding embodiments and contacting the organism with at least one fungicide of the present disclosure. In further embodiments of this aspect, the nucleotide sequence encoding the CGI factor, the CGI factor precursor, the CGI factor fragment, or the CGI factor motif does not occur in the genome of the fungal pathogen.

Still another aspect of the disclosure relates to methods of providing an organism with resistance to a fungal pathogen of the organism, including contacting the organism with the cell of any of the preceding embodiments and contacting the organism with at least one fungicide of the present disclosure. In some embodiments of this aspect, the nucleotide sequence encoding the CGI factor, the CGI factor precursor, the CGI factor fragment, or the CGI factor motif does not occur in the genome of the fungal pathogen.

In some embodiments, the methods of the present disclosure provide resistance to a fungal pathogen selected from the group consisting of Aspergillus, Candida, Coccidioides, Histoplasma, or Blastomyces fungus. In some embodiments, the fungal pathogen is a Mucoromycotina fungus, a Candida species (e.g., C. albicans, C. tropicalis, C. krusei, C. glabrata, and C. pseudotropicalis), an Aspergillus species (e.g., A. fumigatus, A. flavus, and A. niger), Magnaporthe oryzae, Botrytis cinerea, Puccinia spp., Fusarium graminearum, Fusarium oxysporum, Blumeria graminis, Mycosphaerella graminicola, Colletotrichum spp., Ustilago maydis, Melampsora lini, Phakopsora pachyrhizi, or Rhizoctonia solani. In some embodiments, the methods of the present disclosure provide resistance to a fungal pathogen of plants. In some embodiments, the fungal pathogen of plants is Magnaporthe oryzae; Botrytis cinerea; Puccinia spp.; Fusarium graminearum; Fusarium oxysporum; Blumeria graminis; Mycosphaerella graminicola; Colletotrichum spp.; Ustilago maydis; Melampsora lini; Phakopsora pachyrhizi; or Rhizoctonia solani.

### Methods of Controlling or Preventing Fungal Growth

A further aspect of the disclosure relates to methods of controlling a fungal pathogen, including delivering or applying to the fungal pathogen or an environment thereof an antifungal composition of any one of the embodiments described herein.

An additional aspect of the disclosure relates to methods of controlling growth or reproduction of a fungus, including providing the fungus with a composition including an effective amount of at least one conidial germination-inhibiting (CGI) factor, CGI factor precursor, CGI factor fragment, or CGI factor motif, wherein the CGI factor includes an amino acid sequence that has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with a sequence selected from the group of SEQ ID NO: 961-1920, SEQ ID NO: 1957-2189, SEQ ID NO: 2194-2210, SEQ ID NO: 2215-2243, SEQ ID NO: 2457-3361, SEQ ID NO: 5707-5731, and SEQ ID NO: 5755-5911, or wherein the CGI factor motif includes at least one of SEQ ID NO: 1921-1956, a fungicide that interferes with fungal cell wall or fungal cell membranes, and, optionally, an agriculturally or pharmaceutically acceptable carrier, and wherein the amino acid sequence of the CGI factor is not that of an alpha pheromone natively expressed by the fungus, or wherein the nucleotide sequence of the at least one CGI factor, CGI factor precursor, CGI factor fragment, or CGI factor motif does not occur in the genome of the fungus. The nucleotide sequence encoding the at least one CGI factor can include SEQ ID NO: 1-960 or 3362-5698. In some embodiments of this aspect, the CGI factor, the CGI factor precursor, the CGI factor fragment, or the CGI factor motif is active and/or toxic. In further embodiments of this aspect, which can be combined with any preceding embodiment, the composition is provided to the fungus by directly contacting the fungus with the composition, or by delivering the composition to the environment of the fungus.

Yet another aspect of the disclosure relates to methods of preventing growth of a fungus on a surface, including treating the surface with an antifungal composition of any of the embodiments described herein. In some embodiments of this aspect, the CGI factor, the CGI factor precursor, the CGI factor fragment, or the CGI factor motif is active and/or toxic. In further embodiments of this aspect, which can be combined with any preceding embodiment, the surface is a non-living surface or is the surface of a living organism. In additional embodiments of this aspect, which can be combined with any preceding embodiment, the nucleotide sequence of the at least one CGI factor, CGI factor precursor, CGI factor fragment, or CGI factor motif does not occur in the genome of the fungus.

Also provided herein is a method of preventing growth of a fungus on a surface or within a structure (e.g., a human-built structure or artifact). In some embodiments, the method comprises treating the surface or structure with a composition (e.g., a paint, coating, spray, or dip) comprising an antifungal composition of any of the embodiments described herein. In some embodiments, the DNA sequence of the at least one CGI factor, CGI factor precursor, CGI factor fragment, or CGI factor motif does not occur in the genome of the fungus.

In some embodiments, the surface is a non-living surface. In some embodiments, the surface is a surface of a living organism. In embodiments, the structure is a human-built structure or artifact, such as a building, fence, wall, furniture, fabric, or components thereof.

### Methods of Treating a Fungal Disease

A further aspect of the disclosure relates to methods of preventing or reducing disease caused by a fungal pathogen of a plant including contacting one or more cells of the plant with an antifungal composition including the CGI factor, the CGI factor precursor, the CGI factor fragment, or the CGI factor motif of any of the preceding embodiments, a fungicide that interferes with fungal cell wall or fungal cell membranes, and an agriculturally acceptable carrier, whereby disease caused by the fungal pathogen is prevented or decreased in the plant, relative to a control plant not contacted with the antifungal composition.

A further aspect of the disclosure relates to methods treating a subject with a fungal disease including administering to a subject an antifungal or fungicidal composition including the CGI factor, the CGI factor precursor, the CGI factor fragment, or the CGI factor motif of any of the preceding embodiments, fungicide that interferes with fungal cell wall or fungal cell membranes, and a pharmaceutically acceptable carrier, whereby the fungal disease is prevented or decreased in the subject, relative to a control subject not provided with the antifungal composition. In some embodiments of this aspect, the subject is a mammal; in other embodiments the subject is a vertebrate such as a bird, reptile, fish, or amphibian, or is an invertebrate such as an insect. In additional embodiments of this aspect, the mammal is a human. In further embodiments of this aspect, the mammal is a domestic animal or livestock. In still further embodiments of this aspect, which can be combined with any preceding embodiment, the fungal disease is caused by a fungal pathogen selected from the group of Aspergillus, Candida, Coccidioides, Histoplasma, Cryptococcus, Pneumocystis, or Blastomyces fungus. In some embodiments, the fungal disease is an infection of a Mucoromycotina fungus, a Candida species (e.g., C. albicans, C. auris, C. tropicalis, C. krusei, C. glabrata, C. parapsilosis, and C. pseudotropicalis), a Coccidioides species (e.g., C. immitis or C. posadasii), an Aspergillus species (e.g., A. fumigatus, A. flavus, and A. niger), a Mucor species, a Rhizomucor species, a Malassezia species (e.g., M. furfur, M. globose, and M. restricta), Magnaporthe oryzae, Botrytis cinerea, Puccinia spp., Fusarium graminearum; Fusarium oxysporum, Blumeria graminis, Mycosphaerella graminicola, Colletotrichum spp., Ustilago maydis; Melampsora lini, Phakopsora pachyrhizi, or Rhizoctonia solani. In yet another embodiment of this aspect, which can be combined with any preceding embodiment, the fungal disease is aspergillosis, blastomycosis, candidiasis, coccidioidomycosis, histoplasmosis, mucormycosis, mycetoma, ringworm, sporotrichosis, paracoccidioidomycosis, talaromycosis, chromoblastomycosis fusariosis, emergomycosis, scedosporiosis, or fungal meningitis. In additional embodiments of this aspect, which can be combined with any preceding embodiments, the antifungal or fungicidal composition is administered intravenously, intramuscularly, subcutaneously, topically, orally, transdermally, intraperitoneally, intraorbitally, by implantation, by inhalation, intrathecally, intraventricularly, or intranasally.

As used herein, the term “subject” refers to an organism, such as an animal, plant, or microbe. In some embodiments, the subject is a mammal. In some embodiments, the mammal is a human. In other embodiments, the subject is a domestic animal or livestock. In some embodiments, the subject is a non-mammal. In some embodiments, the non-mammal is a reptile, an insect, an amphibian, a bird, or a fish. In embodiments, the subject is a vertebrate animal (e.g., mammal, bird, cartilaginous or bony fish, reptile, or amphibian). In embodiments, the subject is a human; including adults and non-adults (infants and children). In embodiments, the subject is a non-human mammal, such as a non-human primate (e.g., monkeys, apes), ungulate (e.g., cattle, buffalo, bison, sheep, goat, pig, camel, llama, alpaca, deer, horses, donkeys), carnivore (e.g., dog, cat), rodent (e.g., rat, mouse), or lagomorph (e.g., rabbit). In embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots). In embodiments, the subject is an invertebrate such as an arthropod (e.g., insects, arachnids, crustaceans), a nematode, an annelid, a helminth, or a mollusc. In embodiments, the subject is an organism that is part of a symbiosis, such as part of the microbiome of an animal or a plant. In certain optional embodiments, the methods disclosed herein are not methods for treatment of the human or animal body by surgery or therapy. In certain optional embodiments, the subject to which the antifungal composition is applied or provided is not a human, human tissue, human embryo, or human cell in any stage of formation and development. In embodiments, the subject is a plant, such as an angiosperm plant (which can be a dicot or a monocot) or a gymnosperm plant (e.g., a conifer, a cycad, a gnetophyte, a Ginkgo), a fern, horsetail, clubmoss, or a bryophyte. In embodiments, the subject is a eukaryotic alga (unicellular or multicellular). In embodiments, the subject is a plant of agricultural or horticultural importance, such as row crop plants, fruit-producing plants and trees, vegetables, trees, and ornamental plants including ornamental flowers, shrubs, trees, groundcovers, and turf grasses. Plants and plant cells are of any species of interest, including dicots and monocots. Plants of interest include row crop plants, fruit-producing plants and trees, vegetables, trees, and ornamental plants including ornamental flowers, shrubs, trees, groundcovers, and turf grasses.

### Enumerated Embodiments

1. A recombinant DNA construct comprising:
a heterologous promoter operably linked to a nucleic acid molecule comprising a nucleotide sequence that encodes a conidial germination-inhibiting (CGI) factor, a CGI factor precursor, or a CGI factor fragment,
wherein the nucleotide sequence
   (a) encodes at least one CGI factor comprising an amino acid sequence that has at least 80% sequence identity with at least one of SEQ ID NO: 961-1920, SEQ ID NO: 1957-2189, SEQ ID NO: 2194-2210, SEQ ID NO: 2215-2243, SEQ ID NO: 2457-3361, SEQ ID NO: 5707-5731, and SEQ ID NO: 5755-5911, at least one CGI factor precursor, or at least one CGI factor fragment,
   (b) is a synthetic sequence of (a) that has codons optimized for heterologous expression; or
   (c) encodes at least one CGI factor motif.
      2. The recombinant DNA construct of embodiment 2, wherein the CGI factor motif comprises at least one of SEQ ID NO: 1921-1956.
      3. The recombinant DNA construct of embodiment 1 or embodiment 2, wherein the CGI factor, CGI factor precursor, CGI factor fragment, or CGI factor motif is active and/or toxic.
      4. The recombinant DNA construct of any one of embodiments 1-3, wherein the recombinant DNA construct comprises (a) at least one copy of a CGI factor, (b) at least one copy each of two or more CGI factors, (c) at least one CGI factor precursor, (d) at least one CGI factor fragment, (e) at least one CGI factor motif, or (f) any combination of (a) to (e).
      5. The recombinant DNA construct of any one of embodiments 1-4, wherein the heterologous promoter is a bacterial promoter, a fungal promoter, an algal promoter, an animal promoter, or a plant promoter.
      6. The recombinant DNA construct of any one of embodiments 1-5, wherein the heterologous promoter is a plant expressible promoter.
      7. The recombinant DNA construct of embodiment 6, wherein the plant expressible promoter is selected from the group of promoters consisting of a ubiquitin promoter, a cestrum yellow virus promoter, a corn TrpA promoter, a OsMADS 6 promoter, a maize H3 histone promoter, a corn sucrose synthetase 1 promoter, a corn alcohol dehydrogenase 1 promoter, a corn heat shock protein promoter, a maize mtl promoter, a pea small subunit RuBP carboxylase promoter, a rice actin promoter, a rice cyclophilin promoter, a Ti plasmid mannopine synthase promoter, a Ti plasmid nopaline synthase promoter, a Petunia chalcone isomerase promoter, a bean glycine rich protein 1 promoter, a potato patatin promoter, a lectin promoter, a CaMV 35S promoter, and a S-E9 small subunit RuBP carboxylase promoter.
      8. The recombinant DNA construct of any one of embodiments 1-7, wherein the recombinant DNA construct further comprises a nucleotide sequence encoding at least one secretion signal peptide functional in a cell.
      9. A method of providing an organism with resistance to a fungal pathogen of the organism, comprising expressing in a cell of the organism the recombinant DNA construct of any one of embodiments 1-8, wherein the heterologous promoter is functional in the organism.
      10. The method of embodiment 9, wherein the nucleotide sequence encoding the CGI factor, the CGI factor precursor, the CGI factor fragment, or the CGI factor motif does not occur in the genome of the fungal pathogen.
      11. The method of embodiment 9, wherein the recombinant DNA construct is introduced into the cell of the organism by (a) transfection; (b) by inheritance from a parent cell; or (c) by fusion with a donor cell comprising the recombinant DNA construct.
      12. The method of embodiment 9, wherein the organism is a plant, and wherein the recombinant DNA construct is provided to the plant by (a) transformation, or (b) inheritance from at least one parent plant that contained the recombinant DNA construct.
      13. The method of embodiment 12, wherein the plant is selected from the group consisting of a maize plant, a soybean plant, a wheat plant, a rice plant, a cotton plant, a potato plant, a tomato plant, a Brassica spp. plant, and a sugar beet plant.
      14. A CGI factor, a CGI factor precursor, a CGI factor fragment, or a CGI factor motif encoded by the recombinant DNA construct of any one of embodiments 1-8.
      15. An antifungal or fungicidal composition comprising (i) at least one of the CGI factor, the CGI factor precursor, the CGI factor fragment, or the CGI factor motif of embodiment 14, (ii) at least one of a fungicide, e.g., as described herein, and (iii) an agriculturally acceptable carrier.
      16. The antifungal or fungicidal composition of embodiment 15, wherein the composition is formulated as one of a seed treatment, a foliar spray treatment, a foliar drench treatment, a Ready-To-Use (RTU) formulation, a produce coating, a suspension concentrate, a tank-mix, an aerosol, a root dip, a drench, a fog, a soil treatment, an irrigation formulation, or a sprinkler formulation.
      17. The antifungal or fungicidal composition of embodiment 15, wherein the agriculturally acceptable carrier comprises a solid carrier, a liquid carrier, a gel carrier, a suspension, or an emulsion.
      18. The antifungal or fungicidal composition of embodiment 15, wherein the agriculturally acceptable carrier comprises one or more of an adjuvant, an inert component, a dispersant, a surfactant, a tackifier, a binder, or a stabilizer.
      19. A recombinant vector comprising the recombinant DNA construct of any one of embodiments 1-8.
      20. The vector of embodiment 19, wherein the vector comprises a left T-DNA border and a right T-DNA border flanking the recombinant DNA construct.
      21. The vector of embodiment 19, wherein the vector is a viral vector.
      22. A method of providing an organism with resistance to a fungal pathogen of the organism, comprising contacting the organism with the vector of embodiment 19.
      23. The method of embodiment 22, wherein the nucleotide sequence encoding the CGI factor, the CGI factor precursor, the CGI factor fragment, or the CGI factor motif does not occur in the genome of the fungal pathogen.
      24. An RNA transcript resulting from the transcription of the recombinant DNA construct of any one of embodiments 1-8.
      25. A transgenic cell comprising the recombinant vector of embodiment 19.
      26. The transgenic cell of embodiment 25, wherein the cell is selected from a bacterial cell, a fungal cell, an algal cell, an animal cell, or a plant cell.
      27. The transgenic cell of embodiment 26, wherein the transgenic cell is a plant cell.
      28. The transgenic cell of embodiment 27, wherein the plant cell is a dicot plant cell.
      29. The transgenic cell of embodiment 28, wherein the dicot plant cell is selected from the group consisting of a soybean cell, a sunflower cell, a tomato cell, a Brassica spp. cell, a cotton cell, a sugar beet cell, and a tobacco cell.
      30. The transgenic cell of embodiment 27, wherein the plant cell is a monocot plant cell.
      31. The transgenic cell of embodiment 30, wherein the monocot plant cell is selected from the group consisting of a barley cell, a maize cell, an oat cell, a rice cell, a sorghum cell, a sugar cane cell, and a wheat cell.
      32. The transgenic cell of any one of embodiments 25-31, wherein the CGI factor, the CGI factor precursor, the CGI factor fragment, or the CGI factor motif is (a) transiently expressed, or (b) stably expressed.
      33. A method of providing an organism with resistance to a fungal pathogen of the organism, comprising contacting the organism with the cell of embodiment 25.
      34. The method of embodiment 33, wherein the nucleotide sequence encoding the CGI factor, the CGI factor precursor, the CGI factor fragment, or the CGI factor motif does not occur in the genome of the fungal pathogen.
      35. A transgenic plant comprising the transgenic plant cell of embodiment 27.
      36. The transgenic plant of embodiment 35, wherein the transgenic plant is a dicot plant.
      37. The transgenic plant of embodiment 36, wherein the dicot plant is selected from the group consisting of a soybean plant, a sunflower plant, a tomato plant, a Brassica spp. plant, a cotton plant, a sugar beet plant, and a tobacco plant.
      38. The transgenic plant of embodiment 35, wherein the transgenic plant is a monocot plant.
      39. The transgenic plant of embodiment 38, wherein the monocot plant is selected from the group consisting of a barley plant, a maize plant, an oat plant, a rice plant, a sorghum plant, a sugar cane plant, and a wheat plant.
      40. The transgenic plant of embodiment 35, wherein the plant has improved resistance to the fungal pathogen, in comparison to a control plant that does not comprise the transgenic plant cell.
      41. The transgenic plant of embodiment 39, wherein the fungal pathogen is one of an Aspergillus species; Magnaporthe oryzae; Botrytis cinerea; a Puccinia species.; Fusarium graminearum; Fusarium oxysporum; Blumeria graminis; Mycosphaerella graminicola; a Colletotrichum species; Ustilago maydis; Melampsora lini, Phakopsora pachyrhizi, or Rhizoctonia solani.
      42. The transgenic plant of any one of embodiments 35-41, wherein the nucleotide sequence encoding the CGI factor, the CGI factor precursor, the CGI factor fragment, or the CGI factor motif does not occur in the genome of the fungal pathogen.
      43. A transgenic seed of the transgenic plant of embodiment 35, wherein said seed comprises the recombinant DNA construct.
      44. An F1 progeny plant having as at least one parent the transgenic plant of embodiment 35, wherein the F1 progeny plant comprises the recombinant DNA construct.
      45. A harvested product produced from the transgenic plant of embodiment 35, wherein the harvested product comprises the recombinant DNA construct.
      46. The harvested product of embodiment 45, wherein the harvested product is a fruit, a leaf, a stem, a flower, a root, a tuber, or a seed.
      47. A method of producing a disease-resistant plant, comprising:
introducing into a plant the recombinant DNA construct of any one of embodiments 1-8, wherein the CGI factor, CGI factor precursor, the CGI factor fragment, or the CGI factor motif is expressed in the plant;
editing the plant to express a CGI factor comprising an amino acid sequence that has at least 80% sequence identity with at least one of SEQ ID NO: 961-1920, SEQ ID NO: 1957-2189, SEQ ID NO: 2194-2210, SEQ ID NO: 2215-2243, SEQ ID NO: 2457-3361, SEQ ID NO: 5707-5731, and SEQ ID NO: 5755-5911, a CGI factor precursor, or a CGI factor fragment; or
editing the plant to express a CGI factor motif,
thereby producing a disease-resistant transgenic plant that is resistant to diseases caused by a fungus or oomycete.
   48. The method of embodiment 47, wherein the CGI factor motif comprises at least one of SEQ ID NO: 1921-1956.
   49. The method of embodiment 47 or embodiment 48, wherein the CGI factor, CGI factor precursor, CGI factor fragment, or CGI factor motif inhibits growth of and/or is toxic to the fungus or oomycete.
   50. The method of any one of embodiments 47-49, wherein editing the plant is performed using zinc finger-nucleases (ZFNs), transcription activator-like effector nucleases (TALENs), oligonucleotide-directed mutagenesis (ODM), or a clustered regularly interspaced short palindromic repeats (CRISPR)/Cas nuclease.
   51. The method of any one of embodiments 47-49, wherein the introducing step is achieved by (a) transforming the plant; or (b) crossing a first plant comprising the recombinant DNA construct with a second plant.
   52. The method of any one of embodiments 47-49, wherein the introducing step comprises transforming the plant, and wherein transforming the plant comprises bacterially mediated transformation, micro-projectile-mediated transformation, sonication, electroporation, nanoparticle-mediated transformation, or liposome- or spheroplast-mediated vector delivery.
   53. The method of any one of embodiments 47-52, wherein the plant is a maize plant, a soybean plant, a wheat plant, a rice plant, a cotton plant, a potato plant, a tomato plant, a Brassica spp. plant, or a sugar beet plant.
   54. A method of controlling a fungal pathogen, comprising delivering to the fungal pathogen or an environment thereof a composition comprising: (i) an effective amount of at least one of the CGI factor, the CGI factor precursor, the CGI factor fragment, or the CGI factor motif of embodiment 14; and at least one fungicide, e.g., as described herein.
   55. An antifungal composition comprising
(a) an effective amount of at least one conidial germination-inhibiting (CGI) factor, CGI factor precursor, CGI factor fragment, or CGI factor motif, and that comprises
   (i) a polypeptide comprising an amino acid sequence that has at least 80% sequence identity with a sequence selected from the group consisting of SEQ ID NO: 961-1920, SEQ ID NO: 1957-2189, SEQ ID NO: 2194-2210, SEQ ID NO: 2215-2243, SEQ ID NO: 2457-3361, SEQ ID NO: 5707-5731, and SEQ ID NO: 5755-5911; or
   (ii) a polypeptide comprising the amino acid sequence of a CGI factor motif; and
(b) at least one fungicide, e.g., as described herein; and
(c) a carrier.
   56. The antifungal composition of embodiment 53, wherein the CGI factor motif comprises at least one of SEQ ID NO: 1921-1956.
   57. The antifungal composition of embodiment 55 or embodiment 56, wherein the CGI factor, CGI factor precursor, CGI factor fragment, or CGI factor motif inhibits growth of and/or is toxic to a fungus or oomycete.
   58. The antifungal composition of any one of embodiments 55-57, wherein the carrier is selected from an agriculturally acceptable carrier, or a pharmaceutically acceptable carrier.
   59. The antifungal composition of any one of embodiments 55-58, wherein the composition is formulated as a liquid, a gel, an emulsion, a suspension, an encapsulation, a solid, a powder, a coating, a spray, a soil drench, granules, a seed coat, or a bait.
   60. A method of providing an organism with resistance to a fungal pathogen of the organism, comprising contacting the organism with the antifungal composition of any one of embodiments 55-59.
   61. The method of embodiment 60, wherein the nucleotide sequence encoding the CGI factor, the CGI factor precursor, the CGI factor fragment, or the CGI factor motif does not occur in the genome of the fungal pathogen.
   62. A method for controlling a fungal pathogen, the method comprising:
applying, to the fungal pathogen or a locus containing the fungal pathogen, a composition comprising a conidial germination-inhibiting (CGI) factor, a CGI factor precursor, a CGI factor fragment, or a CGI factor motif derived from a least one of the fungal pathogen, a fungus in the same genus as the fungal pathogen, a fungus in a different genus than the fungal pathogen, or a mixture thereof, and at least one fungicide, e.g., as described herein.
   63. The method of embodiment 62, wherein the CGI factor has an amino acid sequence selected from the group consisting of SEQ ID NO: 961-1920, SEQ ID NO: 1957-2189, SEQ ID NO: 2194-2210, SEQ ID NO: 2215-2243, SEQ ID NO: 2457-3361, SEQ ID NO: 5707-5731, and SEQ ID NO: 5755-5911, or an amino acid sequence having at least 80% sequence identity thereto; or wherein the CGI factor motif comprises at least one of SEQ ID NO: 1921-1956.
   64. The method of embodiment 62 or embodiment 63, wherein the CGI factor, the CGI factor precursor, the CGI factor fragment, or the CGI factor motif is active and/or toxic.
   65. A plant having a genome that is modified to express a heterologous DNA sequence that encodes a polypeptide comprising at least one conidial germination-inhibiting (CGI) factor, CGI factor precursor, CGI factor fragment, or CGI factor motif optionally fused to at least one plant secretion signal peptide, wherein the CGI factor, the CGI factor precursor, the CGI factor fragment, or the CGI factor motif inhibits conidial germination, growth, or reproduction of a fungal pathogen of the plant and wherein the CGI factor comprises an amino acid sequence that has at least 80% sequence identity with a sequence selected from the group consisting of SEQ ID NO: 961-1920, SEQ ID NO: 1957-2189, SEQ ID NO: 2194-2210, SEQ ID NO: 2215-2243, SEQ ID NO: 2457-3361, SEQ ID NO: 5707-5731, and SEQ ID NO: 5755-5911, or wherein the CGI factor motif comprises at least one of SEQ ID NO: 1921-1956, and wherein the plant has improved resistance to the fungal pathogen, in comparison to a control plant that does not express the heterologous DNA sequence.
   66. The plant of embodiment 65, wherein the nucleotide sequence of the at least one CGI factor, CGI factor precursor, CGI factor fragment, or CGI factor motif does not occur in the genome of the fungal pathogen.
   67. The plant of embodiment 65 or embodiment 66, wherein the fungal pathogen is one of an Aspergillus species; Magnaporthe oryzae; Botrytis cinerea; a Puccinia species.; Fusarium graminearum; Fusarium oxysporum; Blumeria graminis; Mycosphaerella graminicola; a Colletotrichum species; Ustilago maydis; Melampsora lini, Phakopsora pachyrhizi, or Rhizoctonia solani.
   68. A plant comprising a cell containing a recombinant DNA construct for expressing a CGI factor, a CGI factor precursor, a CGI factor fragment, or a CGI factor motif and comprising:
a heterologous promoter that is functional in the cell and is operably linked to a nucleic acid molecule comprising (a) a nucleotide sequence that encodes at least one conidial germination-inhibiting (CGI) factor, CGI factor precursor, CGI factor fragment, or CGI factor motif, and (b) a nucleotide sequence encoding at least one secretion signal peptide functional in the cells;
wherein the CGI factor comprises an amino acid sequence that has at least 80% sequence identity with a sequence selected from the group consisting of SEQ ID NO: 961-1920, SEQ ID NO: 1957-2189, SEQ ID NO: 2194-2210, SEQ ID NO: 2215-2243, SEQ ID NO: 2457-3361, SEQ ID NO: 5707-5731, and SEQ ID NO: 5755-5911, or wherein the CGI factor motif comprises at least one of SEQ ID NO: 1921-1956; and
wherein the nucleotide sequence encoding the at least one CGI factor, CGI factor precursor, CGI factor fragment, or CGI factor motif optionally includes at least one codon optimized for expression in the cell.
   69. The plant of embodiment 68, wherein the CGI factor, the CGI factor precursor, the CGI factor fragment, or the CGI factor motif is active and/or toxic.
   70. The plant of embodiment 68 or embodiment 69, wherein the cell is a cell of the plant.
   71. The plant of embodiment 68 or embodiment 69, wherein the cell is a bacterial or a fungal cell in or on the plant.
   72. The plant of any one of embodiments 68-71, wherein the plant has improved resistance to the fungal pathogen, in comparison to a control plant that does not comprise the cell.
   73. The plant of embodiment 72, wherein the fungal pathogen is one of an Aspergillus species; Magnaporthe oryzae; Botrytis cinerea; a Puccinia species.; Fusarium graminearum; Fusarium oxysporum; Blumeria graminis; Mycosphaerella graminicola; a Colletotrichum species; Ustilago maydis; Melampsora lini, Phakopsora pachyrhizi, or Rhizoctonia solani.
   74. The plant of any one of embodiments 68-73, wherein the nucleotide sequence encoding the at least one CGI factor, CGI factor precursor, CGI factor fragment, or CGI factor motif does not occur in the genome of the fungal pathogen.
   75. A method of controlling growth or reproduction of a fungus, comprising providing the fungus with a composition comprising (i) an effective amount of at least one conidial germination-inhibiting (CGI) factor, CGI factor precursor, CGI factor fragment, or CGI factor motif, wherein the CGI factor comprises an amino acid sequence that has at least 80% sequence identity with a sequence selected from the group consisting of SEQ ID NO: 961-1920, SEQ ID NO: 1957-2189, SEQ ID NO: 2194-2210, SEQ ID NO: 2215-2243, SEQ ID NO: 2457-3361, SEQ ID NO: 5707-5731, and SEQ ID NO: 5755-5911, or wherein the CGI factor motif comprises at least one of SEQ ID NO: 1921-1956, and wherein the nucleotide sequence of the at least one CGI factor, CGI factor precursor, CGI factor fragment, or CGI factor motif does not occur in the genome of the fungus, and (ii) at least one fungicide, e.g., as described herein.
   76. The method of embodiment 75, wherein the CGI factor, the CGI factor precursor, the CGI factor fragment, or the CGI factor motif is active and/or toxic.
   77. The method of embodiment 75 or embodiment 76, wherein the composition is provided to the fungus by directly contacting the fungus with the composition, or by delivering the composition to the environment of the fungus.
   78. A method of preventing growth of a fungus on a surface, comprising treating the surface with a composition comprising an effective amount of at least one conidial germination-inhibiting (CGI) factor, CGI factor precursor, CGI factor fragment, or CGI factor motif, wherein the CGI factor comprises an amino acid sequence that has at least 80% sequence identity with a sequence selected from the group consisting of SEQ ID NO: 961-1920, SEQ ID NO: 1957-2189, SEQ ID NO: 2194-2210, SEQ ID NO: 2215-2243, SEQ ID NO: 2457-3361, SEQ ID NO: 5707-5731, and SEQ ID NO: 5755-5911, or wherein the CGI factor motif comprises at least one of SEQ ID NO: 1921-1956.
   79. The method of embodiment 78, wherein the CGI factor, the CGI factor precursor, the CGI factor fragment, or the CGI factor motif is active and/or toxic.
   80. The method of 78 or embodiment 79, wherein the surface is a non-living surface or is the surface of a living organism.
   81. The method of any one of embodiments 78-80, wherein the nucleotide sequence of the at least one CGI factor, CGI factor precursor, CGI factor fragment, or CGI factor motif does not occur in the genome of the fungus.
   82. A composition having antifungal properties, comprising a substrate or matrix that is complexed with at least one conidial germination-inhibiting (CGI) factor, CGI factor precursor, CGI factor fragment, or CGI factor motif that is active and/or toxic, wherein the CGI factor comprises an amino acid sequence that has at least 80% sequence identity with a sequence selected from the group consisting of SEQ ID NO: 961-1920, SEQ ID NO: 1957-2189, SEQ ID NO: 2194-2210, SEQ ID NO: 2215-2243, SEQ ID NO: 2457-3361, SEQ ID NO: 5707-5731, and SEQ ID NO: 5755-5911, or wherein the CGI factor motif comprises at least one of SEQ ID NO: 1921-1956.
   83. The composition of embodiment 82, wherein the CGI factor, the CGI factor precursor, the CGI factor fragment, or the CGI factor motif is active and/or toxic.
   84. The composition of embodiment 82 or embodiment 83, wherein the complexation between the substrate or matrix with the at least one CGI factor, CGI factor precursor, CGI factor fragment, or CGI factor motif is through: (a) covalent bonding, (b) non-covalent bonding, or (c) a combination of (a) and (b).
   85. The composition of embodiment 84, wherein the substrate or matrix comprises polypeptides.
   86. The composition of embodiment 84 or embodiment 85, wherein the substrate or matrix comprises self-assembling peptides.
   87. A method of treating a subject with a fungal disease comprising administering to a subject an antifungal or fungicidal composition comprising (i) at least one of the CGI factor, the CGI factor precursor, the CGI factor fragment, or the CGI factor motif of embodiment 13, (ii) at least one fungicide, e.g., as described herein, and (iii) a pharmaceutically acceptable carrier.
   88. The method of embodiment 87, wherein the subject is a mammal.
   89. The method of embodiment 88, wherein the mammal is a human.
   90. The method of embodiment 88, wherein the mammal is a domestic animal or livestock.
   91. The method of any one of embodiments 87-90, wherein the fungal disease is caused by a fungal pathogen selected from the group consisting of Aspergillus, Candida, Coccidioides, Histoplasma, Cryptococcus, Pneumocystis, and Blastomyces fungus.
   92. The method of any one of embodiments 87-91, wherein the fungal disease is aspergillosis, blastomycosis, candidiasis, coccidioidomycosis, histoplasmosis, mucormycosis, mycetoma, ringworm, sporotrichosis, paracoccidioidomycosis, talaromycosis, chromoblastomycosis fusariosis, emergomycosis, scedosporiosis, or fungal meningitis.
   93. The method of any one of embodiments 87-92, wherein the antifungal or fungicidal composition is administered intravenously, intramuscularly, subcutaneously, topically, orally, transdermally, intraperitoneally, intraorbitally, by implantation, by inhalation, intrathecally, intraventricularly, or intranasally.

### EXAMPLES

The presently disclosed subject matter will be better understood by reference to the following Examples, which are provided as exemplary of the invention, and not by way of limitation.

### Example 1

This example describes the spectrum of activity of conidial germination-inhibiting factors (CGI factors).

### Initial Assays Assessing Activity of CGI Factors

### 96-Well Plate Fungal Co-Incubation Assay Protocol

A resazurin plate assay was used to evaluate the inhibitory effects of CGI factors against Botrytis cinerea b05.10, Fusarium graminearum (Gibberella zeae), and Fusarium oxysporum f. sp. lycopersici (Saccardo) Snyder et Hansen CBS 167.30 (Westerdijk Fungal Biodiversity Institute, Netherlands). Each strain was co-incubated with a variety of CGI factors in 50% PDA (potato dextrose broth, Difco). The final concentration of conidia for each strain was 5000 conidia per well of a 96-well plate. Peptides were added at a desired final concentration with 50% PDA comprising the remaining volume up to 100 microliters. After an initial overnight incubation, PrestoBlue Cell Viability Reagent (ThermoFisher Scientific, USA) was added to the plate. For F. graminearum and F. oxysporum, a fluorescence readout was captured using a microplate reader. These fluorescence values correlated to respiration and overall growth of the fungi, so treatments producing lower fluorescence values were deemed effective at inhibiting fungal growth. Plates testing CGI factors against B. cinerea were imaged using an oCelloScope imaging system (Biosense Solutions, Denmark) after 24 hours of incubation at 20° C., and the images thus obtained were manually evaluated to determine which CGI factors inhibited fungal growth. The CGI peptides tested are provided in Table 2.

Pseudoperonospora cubensis (Cucurbit Downy Mildew) Assay Protocol

Pseudoperonospora cubensis (cucurbit downy mildew) sporangia were harvested from leaves of infected cucurbit plants by gently running water over the leaf situated in a funnel within a conical tube so that the resulting suspension collected in the tube. This solution was passed through a 40-micrometer pluriStrainer filter, and sporangia in the filtrate were quantified using a hemocytometer.

The leaf disk assay was carried out in 12-well plates. Each well contained 4 milliliters water agar and a cucurbit leaf disk punched from healthy leaves using a hole punch of the same diameter as the well. Treatments were applied by spraying 1 milliliter of treatment solution using a hand-held spray brush. After plates were dried inside a fume hood, each leaf disc was inoculated with 1 milliliter of sporangia suspension, applied using the hand-held sprayer.

The readout of the cucurbit downy mildew leaf disk assay involved scoring each leaf disc according to a disease severity scale with 4 nominal ranges: 0%, 1-30%, 31-60%, and >60% disease. Discs were visually assessed, approximating sporangia/black structure coverage within the ranges, Treatment effectiveness was evaluated based on its ability to reduce disease severity compared to untreated controls.

TABLE 2
CGI peptides tested in co-incubation assay

| SEQ ID NO | Pep# | Description | Sequence |
|---|---|---|---|
| SEQ ID NO: | Pep105 | F. graminearum | WCTWKGQPCW |
| 2182 | | Alpha- | |
| | | pheromone | |
| SEQ ID NO: | Pep106 | B. cinerea | WCGRPGQPC |
| 2183 | | Alpha- | |
| | | pheromone | |
| SEQ ID NO: | Pep 107 | S. cerevisiae | WHWLQLKPGQPMY |
| 2184 | | Alpha- | |
| | | pheromone | |
| SEQ ID NO: | Pep301 | S. cerevisiae | WHWLQLKPGQPMY |
| 2188 | | Alpha- | WHWLQLKPGQPMY |
| | | pheromone | |
| | | homodimer | |
| SEQ ID NO: | Pep302 | S. cerevisiae | WHWLQLKPGQPMY |
| 5837 | | Alpha- | GGGGWHWLQLKPG |
| | | pheromone | QPMY |
| | | homodimer with | |
| | | GGGG linker | |
| SEQ ID NO: | Pep340 | Sce-octaarginine | WHWLQLKPGQPMY |
| 2240 | | (S. cerevisiae | RRRRRRRR |
| | | Alpha- | |
| | | pheromone | |
| | | fused to cell- | |
| | | penetrating | |
| | | peptide) | |
| SEQ ID NO: | Pep462 | Octaarginine | RRRRRRRR |
| 5747 | | (R8) cell- | |
| | | penetrating | |
| | | peptide (non- | |
| | | CGI peptide | |
| | | CONTROL) | |

### Results

In an initial B, cinerea conidial germination screen of the CGI factors, Pep301, Pep340, and Pep462 completely inhibited conidial germination of the pathogen at least one concentration tested. Pep301 was active at 100 micromolar but not 10 micromolar, whereas Pep340 and Pep462 inhibited B. cinerea at both these concentrations Table 3). Pep107 at 100 microgranular exhibited moderate inhibition of B. cinerea (characterized by partially germinated conidia with hyphal tubes significantly shorter than those of the untreated condition.

These same CGI factors were tested against Pseudo Peronospora cubensis (Downy Mildew) in a leaf disc assay. In this initial assay, Pep107 at 200 micromolar and Pep301 at 20) micromolar reduced the disease severity of leaf discs while Pep340 and Pep462 did not (Table 3). In another assay, all four CGI factors inhibited conidial germination of F. oxysporum at 50 micromolar (Table 3). The same result was observed for F. graminearum except that Pep107 at 50 micromolar did not show any inhibition (Table 3). These results are summarized in Table 4 and indicate that CGI factors inhibit growth of economically significant pathogens, including filamentous fungi and in oomycete, and thus have potential for use in managing fungal disease in crops.

TABLE 3
Effect of CGI factors on fungal growth of B. cinerea,
P. cubensis, F. graminearum, and F. oxysporum

| | | Concentration | |
|---|---|---|---|
| Pathogen | Treatment | (micromolar) | Inhibition |
| Botrytis | Pep105 | 100 | No |
| cinerea | Pep105 | 10 | No |
| | Pep106 | 100 | No |
| | Pep106 | 10 | No |
| | Pep107 | 100 | Some |
| | Pep107 | 10 | No |
| | Pep462 | 100 | Yes |
| | Pep462 | 10 | Yes |
| | Pep340 | 100 | Yes |
| | Pep340 | 10 | Yes |
| | Pep301 | 100 | Yes |
| | Pep301 | 10 | No |
| Pseudoperonospora | Pep107 | 200 | Yes |
| cubensis | Pep301 | 20 | Yes |
| | Pep340 | 20 | No |
| | Pep462 | 20 | No |
| Fusarium | Pep107 | 50 | No |
| graminearum | Pep301 | 50 | Yes |
| | Pep340 | 50 | Yes |
| | Pep462 | 50 | Yes |
| Fusarium | Pep107 | 50 | Yes |
| oxysporum | Pep301 | 50 | Yes |
| | Pep340 | 50 | Yes |
| | Pep462 | 50 | Yes |

TABLE 4
Summary of CGI factor spectrum of activity

| | Pep105 | Pep106 | Pep107 | Pep301 | Pep340 | Pep462 |
|---|---|---|---|---|---|---|
| | (SEQ ID | (SEQ ID | (SEQ ID | (SEQ ID | (SEQ ID | (SEQ ID |
| Pathogen | NO: 2182) | NO: 2183) | NO: 2184) | NO: 2188) | NO: 2240) | NO: 5747) |
| Botrytis | No | No | Yes | Yes | Yes | Yes |
| cinerea | | | | | | |
| Downy | Not | Not | Yes | Yes | No | No |
| Mildew | tested | tested | | | | |
| Fusarium | Not | Not | No | Yes | Yes | Yes |
| graminearum | tested | tested | | | | |
| Fusarium | Not | Not | Yes | Yes | Yes | Yes |
| oxysporum | tested | tested | | | | |

### Example 2

This example describes testing combinations of various CGI peptides and FRAC group 3 fungicides (e.g., triazole fungicides such as Metconazole) for their ability to decrease the growth or reproduction of a fungus.

### Reagents, Fungal Strains, and Growth Conditions

The CGI factors described in Example 1 were tested. Lyophilized peptides were synthesized by Genscript and stored at −20° C. until stocks were prepared. These CGI factor stocks were prepared in MilliQ water at a final concentration of 1 mM. Stocks were aliquoted into cryostorage tubes and stored at −80° C. until use in the assay.

Metconazole was purchased from Sigma Aldrich, USA. Stocks were prepared in 100% DMSO (dimethyl sulfoxide) at a final concentration of 1 mg/mL. Stocks were stored at room temperature until used in the assay.

F. proliferatum isolated from corn seeds was used for all assays. Briefly, corn seeds were soaked in water for at least an hour. The seeds were removed from the water and frozen at −20° C. overnight. They were then placed on plates of Fusarium-selective Nash and Snyder medium and incubated at room temperature. Once cultures had grown, a putative F. proliferatum isolate was chosen based on morphology and propagated on potato dextrose agar (PDA) (Difco, USA) at room temperature. DNA extraction and sequencing was then performed to confirm the isolate to be F. proliferatum.

Conidial stocks of F. proliferatum were prepared from 5 plates of 11-day-old cultures growing on PDA at room temperature. In a biosafety cabinet (BSC) about 3 mL of 0.01% Tween20 was added to each plate. A cell scraper was used to dislodge the conidia into suspension. The resulting liquid from each plate was passed through a 40-micron PluriSelect filter into a sterile 50 mL Falcon tube. Additionally, 0.01% Tween20 and 100% glycerol was added to the solution for a final volume of 20 mL at 20% glycerol. The concentration was quantified using a hemocytometer and was determined to be 1.39*10∂mg/mL. This solution was aliquoted into cryostorage tubes and stored at −80° C. until use in the assay.

The co-incubation assay was performed in 10% sucrose media. This was prepared by mixing 50 g of sucrose in 500 mL sterile water and filter sterilizing the resulting solution through a 0.2-micron filter. The media was stored at room temperature until used in the assay.

Co-Incubation (oCelloScope) Assay

Fungal inhibition assays were performed using the oCelloScope (Biosense Solutions, Denmark) to determine the concentration at which CGI factors and Metconazole demonstrated approximately 100%, 50%, and 0% inhibition of Fusarium proliferatum alone. The concentrations at which these phenotypes are observed are referred to as the treatments high (“H”, 100% inhibition), medium (“M”, 50% inhibition), and low concentrations (“L”, 0% inhibition). In these experiments, a treatment's relative minimum inhibitory concentration (MIC) is its high concentration. The same assay was then used to test CGI factors and Metconazole in various combination types to show a CGI factor's ability to reduce the MIC of Metconazole and other FRAC group 3 triazole fungicides of the same class.

Briefly, F. proliferatum conidia at a final concentration of 10,000 conidia/mL were co-incubated with CGI factor and Metconazole solo or combination treatments in 10% sucrose liquid media to a final volume of 150 microliters in 4 separate wells of a 96 well plate and incubated for 24 hours at 23° C. Images of each well was captured using the oCelloScope, and those images were visually reviewed for fungal growth and conidial germination to determine which conditions demonstrated inhibition of F. proliferatum.

To conduct the oCelloScope assay, all solutions including 10% sucrose media, F. proliferatum conidia stock, Metconazole stocks, and CGI factor stocks were brought to room temperature. Working stocks were prepared by performing dilutions of the original stocks in 10% sucrose. All conditions were prepared in individual microcentrifuge tubes by combining working stocks with media and conidia to achieve the final desired concentrations in a volume of 150 uL per replicate, with at least four replicates per condition. The solutions for each condition were then aliquoted, (150 uL in each well) into a sterile 96-well plate. Plates were incubated at 23° C. for 24 hours. An end point image was taken using the oCelloScope. These images were visually evaluated to determine conditions that demonstrated inhibition of F. proliferatum.

### Results:

### Effect of Fusing a Cell Penetrating Peptide on Activity of CGI Factors

To determine whether a cell penetrating peptide could enhance the activity of antifungal peptides, one CGI factor was tested for activity against F. proliferatum with and without the cell penetrating peptide (CPP) octaarginine. To elucidate any benefit to fusing a CGI factor with a CPP, Pep340 (CGI octaarginine fusion, SEQ ID NO: 2240) and Pep462 (octaarginine, SEQ ID NO: 5747) were tested individually at different concentrations to compare range of activity. Both Pep340 and Pep462 were effective at 1 uM while only Pep340 showed activity at 0.5 uM (Table 5). This result suggests that fusing a CPP such as octaarginine to a CGI factor improves the efficacy of the peptide.

Metconazole in Combination with Different CGI Factors is Effective at Inhibiting Conidial Germination

To test whether CGI factors can be effective at controlling the growth and germination of F. proliferatum in combination with FRAC group 3 triazole fungicides, we paired the CGI factors with metconazole, a representative of this FRAC group and the fungicide class of triazoles. Both metconazole and CGI factor tested at high concentrations inhibited fungal growth by themselves (Table 6). When tested by itself, metconazole did not show any inhibition of conidial germination at the low (0.02 ug/mL) concentration, and wells treated with this composition were characterized by profuse mycelial growth after 24 hr of incubation, similar to the untreated condition. The CGI factor also did not exhibit any reduction in fungal growth at the low concentration when tested individually (Table 6). However, when the low or medium concentrations of metconazole was combined with the low concentration of Pep340, no fungal growth or germination of conidia was observed. These results summarized in Table 7 indicate that CGI factors and Metconazole can work together to inhibit conidial germination and therefore be a potential solution for management of fungal diseases. In addition, a combination of peptides with FRAC group 3 triazole fungicides can reduce the concentration of applied FRAC group 3 triazoles in the field and provide a solution for resistance management for F. proliferatum.

TABLE 5
Inhibition of conidial germination by PEP 340 (CGI-octaarginine
fusion, SEQ ID NO: 2240) and PEP 462 (octaarginine alone,
SEQ ID NO: 5747) at different concentrations.

Inhibition of conidial germination

| Peptide | 1 uM | 0.5 uM | 0.25 uM | 0.125 uM |
|---|---|---|---|---|
| Pep340 | Yes | Yes | No | No |
| Pep462 | Yes | No | No | No |

Metconazole was tested in combination with Pep340 at different concentrations to test cumulative effects on conidial germination of F. proliferatum. Shown in Table 6 are the concentrations of Metconazole and CGI factor tested either alone or in combination along with the effect of the treatment on inhibition of conidial germination observed after 24 hours at 23° C. Table 7 is a results table summarizing observations of growth inhibition of metconazole with different CGI factors.

TABLE 6
Effect of metconazole and CGI combinations
on F. proliferatum conidial germination.

| | | Fungicide | | Peptide | |
|---|---|---|---|---|---|
| Treatment | Fungicide | (ug/ml) | Peptide | (uM) | Inhibition |
| Untreated | — | — | — | — | No |
| Metconazole | Metconazole | 2 | — | — | Yes |
| High | | | | | |
| Metconazole | Metconazole | 0.2 | — | — | No |
| Medium | | | | | |
| Metconazole | Metconazole | 0.02 | — | — | No |
| Low | | | | | |
| Pep340 Low | — | — | Pep340 | 0.125 | No |
| Combination | Metconazole | 0.02 | Pep340 | 0.125 | Yes |
| Pep340 LL | | | | | |
| Combination | Metconazole | 0.2 | Pep340 | 0.125 | Yes |
| Pep340 ML | | | | | |

TABLE 7
Summary of CGI and Metconazole combinations.

| | | | Inhibition in Combination with CGI factors | | | |
|---|---|---|---|---|---|---|
| Fungicide | | | Pep105 | Pep301 | Pep302 | Pep340 |
| | FRAC | | (SEQ ID | (SEQ ID | (SEQ ID | (SEQ ID |
| Name | Group | MOA | NO: 2182) | NO: 2188) | NO: 5837) | NO: 2240) |
| Metconazole | FRAC 3 | Sterol | Not | Awaited | Not | Yes |
| | | biosynthesis | tested | | tested | |

### Example 3

This example describes testing combinations of various CGI peptides and FRAC group 48 fungicides (e.g., polyene macrolide fungicides such as Natamycin) for their ability to decrease the growth or reproduction of a fungus.

### Reagents, Fungal Strains, and Growth Conditions

Lyophilized peptides were synthesized by Genscript and stored at −20° C. until stocks were prepared. These CGI factor stocks were prepared in MilliQ water at a final concentration of 1 mM. Stocks were aliquoted into cryostorage tubes and stored at −80° C. until use in the assay.

Natamycin was purchased from (Cayman Chemical Company, USA). Stocks were prepared in 100% DMSO (dimethyl sulfoxide) at a final concentration of 1 mg/mL. Stocks were stored at room temperature until used in the assay.

F. proliferatum isolated from corn seeds was used for all assays. Briefly, corn seeds were soaked in water for at least an hour. The seeds were removed from the water and frozen at −20° C. overnight. They were then placed on plates of Fusarium-selective Nash and Snyder medium and incubated at room temperature. Once cultures had grown, a putative F. proliferatum isolate was chosen based on morphology and propagated on potato dextrose agar (PDA) (Difco, USA) at room temperature. DNA extraction and sequencing was then performed to confirm the isolate to be F. proliferatum.

Conidial stocks of F. proliferatum were prepared from 5 plates of 11-day-old cultures growing on PDA at room temperature. In a biosafety cabinet (BSC) about 3 mL of 0.01% Tween20 was added to each plate. A cell scraper was used to dislodge the conidia into suspension. The resulting liquid from each plate was passed through a 40-micron PluriSelect filter into a sterile 50 mL Falcon tube. Additionally, 0.01% Tween20 and 100% glycerol was added to the solution for a final volume of 20 mL at 20% glycerol. The concentration was quantified using a hemocytometer and was determined to be 1.39*10∂mg/mL. This solution was aliquoted into cryostorage tubes and stored at −80° C. until use in the assay.

The co-incubation assay was performed in 10% sucrose media. This was prepared by mixing 50 g of sucrose in 500 mL sterile water and filter sterilizing the resulting solution through a 0.2-micron filter. The media was stored at room temperature until used in the assay.

Co-Incubation (oCelloScope) Assay

Fungal inhibition assays were performed using the oCelloScope (Biosense Solutions, Denmark) to determine the concentration at which CGI factors and Natamycin demonstrated approximately 100%, 50%, and 0% inhibition of Fusarium proliferatum alone. The concentrations at which these phenotypes are observed are referred to as the treatments high (“H”, 100% inhibition), medium (“M”, 50% inhibition), and low concentrations (“L”, 0% inhibition). In these experiments, a treatment's relative minimum inhibitory concentration (MIC) is its high concentration. The same assay was then used to test CGI factors and Natamycin in various combination types to show a CGI factor's ability to reduce the MIC of Natamycin and other polyene macrolide fungicides.

Briefly, F. proliferatum conidia at a final concentration of 10,000 conidia/mL were co-incubated with CGI factor and [Natamycin]solo or combination treatments in 10% sucrose liquid media to a final volume of 150 ul in 4 separate wells of a 96 well plate and incubated for 24 hours at 23° C. Images of each well was captured using the oCelloScope, and those images were visually reviewed for fungal growth and conidial germination to determine which conditions demonstrated inhibition of F. proliferatum.

To conduct the oCelloScope assay, all solutions including 10% sucrose media, F. proliferatum conidia stock, Natamycin stocks, and CGI factor stocks were brought to room temperature. Working stocks were prepared by performing dilutions of the original stocks in 10% sucrose. All conditions were prepared in individual microcentrifuge tubes by combining working stocks with media and conidia to achieve the final desired concentrations in a volume of 150 uL per replicate, with at least four replicates per condition. The solutions for each condition were then aliquoted, (150 uL in each well) into a sterile 96-well plate. Plates were incubated at 23° C. for 24 hours. An end point image was taken using the oCelloScope. These images were visually evaluated to determine conditions that demonstrated inhibition of F. proliferatum.

### Results:

### Effect of Fusing a Cell Penetrating Peptide on Activity of CGI Factors

To determine whether a cell penetrating peptide could enhance the activity of antifungal peptides, one CGI factor was tested for activity against F. proliferatum with and without the cell penetrating peptide (CPP) octaarginine. To elucidate any benefit to fusing a CGI factor with a CPP, Pep340 (CGI octaarginine fusion, SEQ ID NO: 2240) and Pep462 (octaarginine, SEQ ID NO: 5747) were tested individually at different concentrations to compare range of activity. Both Pep340 and Pep462 were effective at 1 uM while only Pep340 showed activity at 0.5 uM (Table 8). This result suggests that fusing a CPP such as octaarginine to a CGI factor improves the efficacy of the peptide.

Natamycin in Combination with Different CGI Factors is Effective at Inhibiting Conidial Germination

To test whether CGI factors can be effective at controlling the growth and germination of F. proliferatum in combination with polyene macrolide fungicides, we paired the CGI factors with natamycin, a representative of this class compound. Both natamycin and CGI factor tested at high concentrations inhibited fungal growth by themselves (Table 9). When tested by itself, natamycin did not show any inhibition of conidial germination at the low (5 uM) concentration; wells treated with this composition were characterized by profuse mycelial growth after 24 hr of incubation, similar to the untreated condition. The CGI factors also did not exhibit any reduction in fungal growth at the low and medium concentrations when tested individually (Table 9). However, when the low concentration of natamycin was combined with either low or high concentration of PEP301 (SEQ ID NO: 2188) and PEP302 (SEQ ID NO: 5837), no fungal growth or germination of conidia was observed. The same effect was observed when low concentration of Natamycin was combined with a low concentration of PEP340 (SEQ ID NO: 2240) (Table 9). These results summarized in Table 10 indicate that CGI factors and Natamycin can work together to inhibit conidial germination and therefore be a potential solution for management of fungal diseases. In addition, a combination of peptides with chemical fungicides can reduce the concentration of applied chemical in the field and provide a solution for resistance management for F. proliferatum.

TABLE 8
Inhibition of conidial germination by PEP 340 (CGI-octaarginine
fusion, SEQ ID NO: 2240) and PEP 462 (octaarginine alone,
SEQ ID NO: 5747) at different concentrations.

Inhibition of conidial germination

| Peptide | 1 uM | 0.5 uM | 0.25 uM | 0.125 uM |
|---|---|---|---|---|
| Pep340 | Yes | Yes | No | No |
| Pep462 | Yes | No | No | No |

Natamycin was tested in combination with PEP340, PEP301 and PEP302 at different concentrations to test cumulative effects on conidial germination of F. proliferatum. Shown in Table 9 are the concentrations of Natamycin and CGIs tested either alone or in combination along with the effect of the treatment on inhibition of conidial germination s observed after 24 hours at 23° C. Table 10 is a results table summarizing observations of growth inhibition of Natamycin with different CGI factors.

TABLE 9
Effect of natamycin and CGI combinations on
F. proliferatum conidial germination

| | | Fungicide | | Peptide | |
|---|---|---|---|---|---|
| Treatment | Fungicide | (ug/ml) | Peptide | (uM) | Inhibition |
| Untreated | — | — | — | — | No |
| Natamycin | Natamycin | 100 | — | — | Yes |
| High | | | | | |
| Natamycin | Natamycin | 10 | — | — | No |
| Medium | | | | | |
| Natamycin | Natamycin | 5 | — | — | No |
| Low | | | | | |
| Pep301 Low | — | — | Pep301 | 0.25 | No |
| Pep301 Medium | — | — | Pep301 | 0.5 | No |
| Pep301 High | — | — | Pep301 | 1.0 | Yes |
| Pep302 Low | — | — | Pep302 | 0.25 | No |
| Pep302 Medium | — | — | Pep302 | 0.5 | No |
| Pep302 High | — | — | Pep302 | 1.0 | Yes |
| Pep340 Low | — | — | Pep340 | 0.125 | No |
| Combination | Natamycin | 5 | Pep301 | 0.25 | Yes |
| Pep301 LL | | | | | |
| Combination | Natamycin | 5 | Pep301 | 0.5 | Yes |
| Pep301 LM | | | | | |
| Combination | Natamycin | 5 | Pep302 | 0.25 | Yes |
| Pep302 LL | | | | | |
| Combination | Natamycin | 5 | Pep302 | 0.5 | Yes |
| Pep302 LM | | | | | |
| Combination | Natamycin | 5 | Pep340 | 0.125 | Yes |
| Pep340 LL | | | | | |

TABLE 10
Summary of CGI and Natamycin combinations

| | | | Inhibition in Combination with CGI factor | | | |
|---|---|---|---|---|---|---|
| Fungicide | | | Pep105 | Pep301 | Pep302 | Pep340 |
| | Compound | | (SEQ ID | (SEQ ID | (SEQ ID | (SEQ ID |
| Name | class | MOA | NO: 2182) | NO: 2188) | NO: 5837) | NO: 2240) |
| Natamycin | Polyene | Ergosterol | No | Yes | Yes | Yes |
| | macrolide | binding | | | | |

### Example 4

This example describes testing combinations of various CGI peptides and cell membrane-interacting or cell wall-interacting antifungal peptides (e.g., drosomycin) for their ability to decrease the growth or reproduction of a fungus.

### Reagents, Fungal Strains, and Growth Conditions

Lyophilized peptides were synthesized by Genscript and stored at −20° C. until stocks were prepared. These CGI factor stocks were prepared in MilliQ water at a final concentration of 1 mM. Stocks were aliquoted into cryostorage tubes and stored at −80° C. until used in the assay.

Drosomycin is a 44-residue antifungal peptide with four intramolecular disulfide bridges, including a terminal cysteine, and was originally described from Drosophila flies; see Feldbaum et al. (1994) J. Biol Chem., 269:33159-33163. Drosomycin's sequence has homology with antifungal peptides identified from plants such as members of the Brassicaceae (Feldbaum et al. (1994)). Drosomycin-01 was heterologously expressed and detected as previously described, with modifications; see Yuan et al. (2006) Protein Expr. Purif., 52:457-462; doi: 10.1016/j.pep.2006.10.024. The coding sequence (Yuan et al. (2006)) was codon-optimized via De Novo DNA and cloned as an N-terminal translational fusion to glutathione S-transferase (GST), and the resulting plasmid transformed into E. coli cells, which were grown out into a 1-L culture. The culture was harvested and centrifuged. The cell pellet was frozen at −80° C., then thawed and lysed. The GST-Drosomycin-01 was purified from the cell lysate using a GST trapping column, on-column digestion was performed to remove the GST tag, and the eluted tagless drosomycin-01 was further purified by HPLC on a C18 reverse-phase column.

F. proliferatum isolated from corn seeds was used for all assays. Briefly, corn seeds were soaked in water for at least an hour. The seeds were removed from the water and frozen at −20° C. overnight. They were then placed on plates of Fusarium-selective Nash and Snyder medium and incubated at room temperature. Once cultures had grown, a putative F. proliferatum isolate was chosen based on morphology and propagated on potato dextrose agar (PDA) (Difco, USA) at room temperature. DNA extraction and sequencing was then performed to confirm the isolate to be F. proliferatum.

Conidial stocks of F. proliferatum were prepared from 5 plates of 11-day-old cultures growing on PDA at room temperature. In a biosafety cabinet (BSC) about 3 mL of 0.01% Tween20 was added to each plate. A cell scraper was used to dislodge the conidia into suspension. The resulting liquid from each plate was passed through a 40-micron PluriSelect filter into a sterile 50 mL Falcon tube. Additionally, 0.01% Tween20 and 100% glycerol was added to the solution for a final volume of 20 mL at 20% glycerol. The concentration was quantified using a hemocytometer and was determined to be 1.39*10∂mg/mL. This solution was aliquoted into cryostorage tubes and stored at −80° C. until used in the assay.

The co-incubation assay was performed in 10% sucrose media. This was prepared by mixing 50 g of sucrose in 500 mL sterile water and filter sterilizing the resulting solution through a 0.2-micron filter. The media was stored at room temperature until used in the assay.

Co-Incubation (oCelloScope) Assay.

Fungal inhibition assays were performed using the oCelloScope (Biosense Solutions, Denmark) to determine the concentration at which CGI factors and Drosomycin demonstrated approximately 100%, 50%, and 0% inhibition of Fusarium proliferatum alone. The concentrations at which these phenotypes are observed are referred to as the treatments high (“H”, 100% inhibition), medium (“M”, 50% inhibition), and low concentrations (“L”, 0% inhibition). In these experiments, a treatment's relative minimum inhibitory concentration (MIC) is its high concentration. The same assay was then used to test CGI factors and Drosomycin in various combination types to show a CGI factor's ability to reduce the MIC of fungal respiration inhibitors.

Briefly, F. proliferatum conidia at a final concentration of 10,000 conidia/mL were co-incubated with CGI factor and Drosomycin solo or combination treatments in 10% sucrose liquid media to a final volume of 150 ul in 4 separate wells of a 96 well plate and incubated for 24 hours at 23° C. Images of each well was captured using the oCelloScope, and those images were visually reviewed for fungal growth and conidial germination to determine which conditions demonstrated inhibition of F. proliferatum.

To conduct the oCelloScope assay, all solutions including 10% sucrose media, F. proliferatum conidia stock, Drosomycin stocks, and CGI factor stocks were brought to room temperature. Working stocks were prepared by performing dilutions of the original stocks in 10% sucrose. All conditions were prepared in individual microcentrifuge tubes by combining working stocks with media and conidia to achieve the final desired concentrations in a volume of 150 uL per replicate, with at least four replicates per condition. The solutions for each condition were then aliquoted, (150 uL in each well) into a sterile 96-well plate. Plates were incubated at 23° C. for 24 hours. An end point image was taken using the oCelloScope. These images were visually evaluated to determine conditions that demonstrated inhibition of F. proliferatum.

### Results:

### Effect of Fusing a Cell Penetrating Peptide on Activity of CGI Factors

To determine whether a cell penetrating peptide could enhance the activity of antifungal peptides, one CGI factor was tested for activity against F. proliferatum with and without the cell penetrating peptide (CPP) octaarginine. To elucidate any benefit to fusing a CGI factor with a CPP, Pep340 (CGI octaarginine fusion) and Pep462 (octaarginine) were tested individually at different concentrations to compare range of activity. Both Pep340 and Pep462 were effective at 1 uM while only Pep340 showed activity at 0.5 uM (Table 11). This result suggests that fusing a CPP such as octaarginine to a CGI factor improves the efficacy of the peptide.

Drosomycin in Combination with Different CGI Factors is Effective at Inhibiting Conidial Germination

To test whether CGI factors can be effective at controlling the growth and germination of F. proliferatum in combination with a cell membrane-interacting or cell wall-interacting antifungal peptide, we paired the CGI factors with Drosomycin, representative of this mode of action. When tested by itself, Drosomycin did not show any inhibition of conidial germination at its low and medium concentrations (0.5 uM and 1 uM, respectively); wells treated with these concentrations were characterized by profuse mycelial growth after 24 hr of incubation, similar to the untreated controls. The CGI factors also did not exhibit any reduction in fungal growth at the low and medium concentrations when tested individually (Table 12). However, when the low concentration of Drosomycin was combined with the medium concentration of either Pep105, Pep301, or Pep302, no fungal growth or germination of conidia was observed. The same effect was observed when medium concentration of Drosomycin was combined with a low concentration of Pep340 (Table 12). These results summarized in Table 13 indicate that CGI factors and these cell membrane-interacting or cell wall-interacting antifungal peptides can work together to inhibit conidial germination and therefore be a potential solution for management of fungal diseases. In addition, a combination of CGI peptides with cell membrane-interacting or cell wall-interacting antifungal peptides can reduce the concentration of applied cell membrane-interacting or cell wall-interacting antifungal peptides in the field and provide a solution for resistance management for F. proliferatum.

TABLE 11
Inhibition of conidial germination by Pep340 (CGI-octaarginine
fusion) and octaarginine alone at different concentrations.

Inhibition of conidial germination

| Peptide | 1 uM | 0.5 uM | 0.25 uM | 0.125 uM |
|---|---|---|---|---|
| Pep340 | Yes | Yes | No | No |
| Pep462 | Yes | No | No | No |

Drosomycin was tested in combination with Pep105, Pep301, Pep302, and Pep340 at different concentrations to test cumulative effects on conidial germination of F. proliferatum. Shown in Table 12 are the concentrations of Drosomycin and CGI factors tested either alone or in combination along with the effect of the treatment on inhibition of conidial germination observed after 24 hours at 23° C. Table 13 is a results table summarizing observations of growth inhibition of drosomycin with different CGI factors.

TABLE 12
Effect of Drosomycin and CGI combinations
on F. proliferatum conidial germination.

| | | Fungicide | | Peptide | |
|---|---|---|---|---|---|
| Treatment | Fungicide | (uM) | Peptide | (uM) | Inhibition |
| Untreated | — | — | — | — | No |
| Drosomycin | Drosomycin | 0.5 | — | — | No |
| Low | | | | | |
| Drosomycin | Drosomycin | 1 | — | — | No |
| Medium | | | | | |
| Drosomycin | Drosomycin | 8 | — | — | Yes |
| High | | | | | |
| Pep105 Low | — | — | Pep105 | 10 | No |
| Pep105 | — | — | Pep105 | 50 | No |
| Medium | | | | | |
| Pep105 High | — | — | Pep105 | 100 | Yes |
| Pep301 Low | — | — | Pep301 | 0.25 | No |
| Pep301 | — | — | Pep301 | 0.5 | No |
| Medium | | | | | |
| Pep301 High | — | — | Pep301 | 1.0 | Yes |
| Pep302 Low | — | — | Pep302 | 0.25 | No |
| Pep302 | — | — | Pep302 | 0.5 | No |
| Medium | | | | | |
| Pep302 High | — | — | Pep302 | 1.0 | Yes |
| Pep340 Low | — | — | Pep340 | 0.125 | No |
| Pep340 | — | — | Pep340 | 0.5 | No |
| Medium | | | | | |
| Pep340 High | — | — | Pep340 | 1.0 | Yes |
| Combination | Drosomycin | 0.5 | Pep105 | 50 | Yes |
| Pep105 LM | | | | | |
| Combination | Drosomycin | 0.5 | Pep301 | 0.5 | Yes |
| Pep301 LM | | | | | |
| Combination | Drosomycin | 0.5 | Pep302 | 0.5 | Yes |
| Pep302 LM | | | | | |
| Combination | Drosomycin | 1 | Pep340 | 0.125 | Yes |
| Pep340 ML | | | | | |

TABLE 13
Summary of CGI and Drosomycin combinations.

| Fungicide | | | | | | |
|---|---|---|---|---|---|---|
| | Compound | | Inhibition in Combination with CGIs factors | | | |
| Name | class | MOA | Pep105 | Pep301 | Pep302 | Pep340 |
| Drosomycin | Antifungal | Membrane | Yes | Yes | Yes | Yes |
| | peptides | disruption | | | | |

### Example 5

This example describes testing combinations of various CGI peptides and fungicides that inhibit beta-glucan synthesis in the fungal cell wall. (e.g., cyclic lipopeptide fungicides such as echinocandins, specifically caspofungin) for their ability to decrease the growth or reproduction of a fungus.

### Reagents, Fungal Strains, and Growth Conditions

The CGI factors described in Example 1 are tested. Lyophilized peptides are produced by Genscript and stored at −20′C until stocks are created. These CGI factor stocks are prepared in MilliQ water at a final concentration of 1 mM. Stocks are aliquoted into cryostorage tubes and stored at −80° C. until use in the assay.

The echninocandin fungicide caspofungin is tested. Stocks are stored at room temperature until used in the assay.

To isolate F. proliferatum, corn seeds are soaked in water for at least an hour. The seeds are removed from the water and frozen at −20° C. overnight. They are then placed on plates of Fusarium-selective Nash and Snyder medium. Plates are incubated at room temperature. Once cultures have grown, a putative Fusarium isolate is chosen based on morphology and propagated further on 1× potato dextrose agar (PDA) at room temperature. DNA extraction and sequencing are then performed to confirm the isolate as F. proliferatum.

Conidial stocks of F. proliferatum are prepared from 5 plates of 11-day-old cultures growing on PDA at room temperature. In a biosafety cabinet (BSC) about 3 mL of 0.01% Tween20 are added to each plate. A cell scraper is used to dislodge the conidia into suspension. The resulting liquid from each plate is passed through a 40-micron PluriSelect filter into a sterile 50 mL Falcon tube. Additionally, 0.01% Tween20 and 100% glycerol is added to the solution for a final volume of 20 mL at 20% glycerol. The concentration is quantified using a hemocytometer, determined to be 1.39*10∂mg/mL. This solution is aliquoted into cryostorage tubes and stored at −80° C. until use in the assay.

The media used in this assay is 10% sucrose. This is prepared by combining 50 g of sucrose with 500 mL sterile water, mixing well, then passing through a 0.2-micron filter. Media is stored at room temperature until used in the assay.

Co-Incubation (oCelloScope) Assay

Fungal inhibition assays are performed using the oCelloScope (Biosense Solutions) to determine the concentration at which CGI factors and caspofungin demonstrate approximately 100%, 50%, and 0% inhibition of Fusarium proliferatum alone. The concentrations at which these phenotypes are observed are referred to as the treatments high (“H”, 100% inhibition), medium (“M”, 50% inhibition), and low concentrations (“L”, 0% inhibition). In these experiments, a treatment's relative minimum inhibitory concentration (MIC) is its high concentration. The same assay is then used to test CGI factors and caspofungin in various combination types to show a CGI factor's ability to reduce the MIC of caspofungin and other fungicides of the same anilinopyrimidine class. To elucidate advantageous combinations, three types of combinations at sub-MIC (medium and low concentrations) are tested. For the first combination type, CGI factor and caspofungin are each tested together at their lowest concentrations—this condition is referred to as “low-low (LL)”. The second combination type pairs caspofungin's lowest concentration with the CGI factor's medium concentration—“fungicide low, CGI factor medium (LM)”. Finally, the third combination type pairs caspofungin's medium concentration with the CGI factor's lowest concentration—“fungicide medium, CGI factor low (ML)”

Briefly, F. proliferatum conidia at a final concentration of 10,000 conidia/mL are co-incubated with CGI factor and caspofungin solo or combination treatments in 10% sucrose liquid media within a 96 well plate for about 24 hours at 23° C. Images of each well were taken using the oCelloScope, and those images were visually reviewed to determine which conditions demonstrated inhibition of F. proliferatum.

To conduct the oCelloScope assay, materials are first gathered and brought to room temperature. This includes media, F proliferatum conidia stock, anilinopyrimidine fungicide stocks, CGI factor stocks, and sterile 96 well plate(s). Generally working stocks are prepared by performing dilutions of original stocks in 10% sucrose. All conditions are prepared in individual microcentrifuge tubes—combining working stocks with media and conidia to achieve final desired concentrations in a volume provisioning for 150 uL per replicate, at least four replicates per condition, plus 100 uL extra volume. The solutions for each condition are then aliquoted, 150 uL in each well, into a 96 well plate according to a predetermined plate map. Plates are incubated at 23° C. for at least 24 hours. An end point image is taken using the oCelloScope. These images are visually evaluated to determine which conditions demonstrate inhibition against F. proliferatum.

### Results

Through the oCelloScope assay, a combination is deemed successful if the observed inhibition resulting from the pair is greater than an additive effect, i.e., a combination produces inhibition while the components tested individually do not.

### Example 6

This example describes testing combinations of various CGI peptides and FRAC group 3 fungicides (e.g., triazole fungicides such as Metconazole), FRAC group 48 fungicides (e.g., polyene macrolide fungicides such as Natamycin), cell membrane-interacting or cell wall-interacting antifungal peptides (e.g., drosomycin), or fungicides that inhibit beta-glucan synthesis in the fungal cell wall. (e.g., cyclic lipopeptide fungicides such as echinocandins, specifically caspofungin) for their ability to decrease the growth or reproduction of the cucurbit downy mildew fungus using a leaf disc assay.

Pseudoperonospora cubensis (Cucurbit Downy Mildew) Assay Protocol

The assay is conducted as described in Example 1.

The treatments described in Examples 2, 3, 4, and 5 are tested.

### Example 7

This example describes testing combinations of various CGI peptides and fungal signal transduction-inhibiting fungicides (e.g., phenylpyrrole fungicides such as fludioxonil or fenpiclonil) for their ability to decrease the growth or reproduction of a fungus.

### Reagents, Fungal Strains, and Growth Conditions.

Lyophilized peptides were produced by Genscript and stored at −20° C. until stocks were created. These CGI factor stocks were prepared in MilliQ water at a final concentration of 1 mm. Stocks were aliquoted into cryostorage tubes and stored at −80° C. until use in the assay.

Fenpiclonil and Fludioxonil were acquired from Sigma Aldrich. Fenpiclonil stocks were prepared in 100% acetone at a final concentration of 1 uM. Fludioxonil stocks were prepared in 100% dimethyl sulfoxide (DMSO) at a final concentration of 1 mg/mL. Stocks were stored at room temperature until used in the assay.

To isolate F. proliferatum, corn seeds were soaked in water for at least an hour. The seeds were removed from the water and frozen at −20° C. overnight. They were then placed on plates of Fusarium-selective Nash and Snyder medium. Plates were incubated at room temperature. Once cultures had grown, a putative Fusarium isolate was chosen based on morphology and propagated further on 1× potato dextrose agar (PDA) at room temperature. DNA extraction and sequencing was then performed to confirm the isolate as F. proliferatum.

Conidial stocks of F. proliferatum were prepared from 5 plates of 11-day-old cultures growing on PDA at room temperature. In a biosafety cabinet (BSC) about 3 mL of 0.01% Tween20 was added to each plate. A cell scraper was used to dislodge the conidia into suspension. The resulting liquid from each plate was passed through a 40-micron PluriSelect filter into a sterile 50 mL Falcon tube. Additionally, 0.01% Tween20 and 100% glycerol was added to the solution for a final volume of 20 mL at 20% glycerol. The concentration was quantified using a hemocytometer, determined to be 1.39*10∂mg/mL. This solution was aliquoted into cryostorage tubes and stored at −80° C. until use in the assay.

The media used in this assay was 10% sucrose. This was prepared by combining 50 g of sucrose with 500 mL sterile water, mixing well, then passing through a 0.2-micron filter. Media was stored at room temperature until used in the assay.

Co-Incubation (oCelloScope) Assay.

Fungal inhibition assays were performed using the oCelloScope (Biosense Solutions) to determine the concentration at which CGI factors, Fludioxonil, and Fenpiclonil demonstrated approximately 100%, 50%, and 0% inhibition of Fusarium proliferatum alone. The concentrations at which these phenotypes were observed are referred to as the treatment's high, medium, and low concentrations (H, M, and L, respectively). In these experiments, a treatment's relative minimum inhibitory concentration (MIC) is its high concentration. The same assay was then used to test CGI factors and Fludioxonil or Fenpiclonil in various combination types to show a CGI factor's ability to reduce the MIC of each fungicide and other fungicides of the same phenylpyrrole class.

Briefly, F. proliferatum conidia at a final concentration of 10,000 conidia/mL were co-incubated with CGI factor, Fenpiclonil, and Fludioxonil solo or combination treatments in 10% sucrose liquid media within a 96 well plate for about 24 hours at 23° C. Images of each well were taken using the oCelloScope, and those images were visually reviewed to determine which conditions demonstrated inhibition of F. proliferatum.

To conduct the oCelloScope assay, materials were first gathered and brought to room temperature. This includes media, F. proliferatum conidia stock, phenylpyrrole fungicide stocks, CGI factor stocks, and sterile 96 well plate(s). Generally working stocks were prepared by performing dilutions of original stocks in 10% sucrose. All conditions were prepared in individual microcentrifuge tubes-combining working stocks with media and conidia to achieve final desired concentrations in a volume provisioning for 150 uL per replicate, at least four replicates per condition, plus 100 uL extra volume. The solutions for each condition were then aliquoted, 150 uL in each well, into a 96 well plate according to a predetermined plate map. Plates were incubated at 23° C. for at least 24 hours. An end point image was taken using the oCelloScope. These images were visually evaluated to determine which conditions demonstrated inhibition against F. proliferatum.

### Results

### Effect of Fusing a Cell Penetrating Peptide on Activity of CGI Factors

To determine whether a cell penetrating peptide could enhance the activity of antifungal peptides, one CGI factor was tested for activity against F. proliferatum with and without the cell penetrating peptide (CPP) octaarginine. To elucidate any benefit to fusing a CGI factor with a CPP, Pep340 (CGI octaarginine fusion) and Pep462 (octaarginine) were tested individually at different concentrations to compare range of activity. Both Pep340 and Pep462 were effective at 1 uM while only Pep340 showed activity at 0.5 uM (Table 14). This result suggests that fusing a CPP such as octaarginine to a CGI factor improves the efficacy of the peptide.

Phenylpyrrole Fungicides in Combination with Different CGI Factors are Effective at Inhibiting Conidial Germination

To test whether CGI factors can be effective at controlling the growth and germination of F. proliferatum in combination with fungicides of the phenylpyrrole class, we paired the CGI factors with Fenpiclonil and Fludioxonil, representatives of this class compound and FRAC group 12. Both the phenylpyrrole fungicides and the CGI factors tested individually at high concentrations inhibited fungal growth by themselves (Table 15). When tested by itself, Fludioxonil did not show any inhibition of conidial germination at the low (0.02 ug/mL) concentration; wells with this treatment were characterized by profuse mycelial growth after 24 hr of incubation, similar to the untreated control. Similarly, Fenpiclonil did not inhibit conidial germination at the low (5 uM) concentration. The CGI factors also did not exhibit any reduction in fungal growth at the low and medium concentrations when tested individually (Table 15). However, when the low concentration of each phenylpyrrole fungicide was combined with either low or high concentration of Pep340 (SEQ ID NO: 2240), no fungal growth or germination of conidia was observed. Fludioxonil at its medium concentration (0.2 ug/mL) combined with the low concentration of Pep340 also exhibited complete fungal inhibition. The same effect was observed when the low concentration of Fenpiclonil was combined with medium concentrations of Pep301 (SEQ ID NO: 2188) and Pep302 (SEQ ID NO: 5837) (Table 15). These results summarized in Table 16 indicate that CGI factors and phenylpyrroles can work together to inhibit conidial germination and therefore be a potential solution for management of fungal diseases.

TABLE 14
Inhibition of conidial germination by PEP 340 (CGI-octaarginine
fusion, SEQ ID NO: 2240) and PEP 462 (octaarginine alone,
SEQ ID NO: 5747) at different concentrations.

Inhibition of conidial germination

| Peptide | 1 uM | 0.5 uM | 0.25 uM | 0.125 uM |
|---|---|---|---|---|
| Pep340 | Yes | Yes | No | No |
| Pep462 | Yes | No | No | No |

Fludioxonil and Fenpiclonil were tested in combination with PEP340 at different concentrations to test cumulative effects on conidial germination of F. proliferatum. Fenpiclonil was also tested in combination with Pep301 and Pep302. Shown in Table 15 are the concentrations of the phenylpyrrole fungicides and CGIs tested either alone or in combination along with the effect of the treatment on inhibition of conidial germination observed after 24 hours at 23YC. Table 16 is a results table summarizing observations of growth inhibition of phenylpyrrole fungicides with different CGI factors.

TABLE 15
Effect of phenylpyrrole fungicides and CGI combinations
on F. proliferatum conidial germination.

| | | | | | [Peptide] | |
|---|---|---|---|---|---|---|
| Treatment | Fungicide | [Fungicide] | | Peptide | (uM) | Inhibition |
| Untreated | — | — | | — | — | No |
| Fludioxonil | Fludioxonil | 2 | ug/mL | — | — | Yes |
| High | | | | | | |
| Fludioxonil | Fludioxonil | 0.2 | ug/mL | — | — | No |
| Medium | | | | | | |
| Fludioxonil | Fludioxonil | 0.02 | ug/mL | — | — | No |
| Low | | | | | | |
| Pep340 Low | — | — | | Pep340 | 0.125 | No |
| Pep340 + | Fludioxonil | 0.02 | ug/mL | Pep340 | 0.125 | Yes |
| Fludioxonil LL | | | | | | |
| Pep340 + | Fludioxonil | 0.2 | ug/mL | Pep340 | 0.125 | Yes |
| Fludioxonil ML | | | | | | |
| Fenpiclonil | Fenpiclonil | 50 | uM | — | — | Yes |
| High | | | | | | |
| Fenpiclonil | Fenpiclonil | 10 | uM | — | — | No |
| Medium | | | | | | |
| Fenpiclonil | Fenpiclonil | 5 | uM | — | — | No |
| Low | | | | | | |

| | | | | | |
|---|---|---|---|---|---|
| Pep301 Low | — | — | Pep301 | 0.25 | No |
| Pep301 | — | — | Pep301 | 0.5 | No |
| Medium | | | | | |
| Pep301 High | — | — | Pep301 | 1.0 | Yes |
| Pep302 Low | — | — | Pep302 | 0.25 | No |
| Pep302 | — | — | Pep302 | 0.5 | No |
| Medium | | | | | |
| Pep302 High | — | — | Pep302 | 1.0 | Yes |
| Pep301 + | Fenpiclonil | 5 | Pep301 | 0.5 | Yes |
| Fenpiclonil LM | | | | | |
| Pep302 + | Fenpiclonil | 5 | Pep302 | 0.5 | Yes |
| Fenpiclonil LM | | | | | |
| Pep340 + | Fenpiclonil | 5 | Pep340 | 0.125 | Yes |
| Fenpiclonil LL | | | | | |

TABLE 16
Summary of CGI and Phenylpyrrole fungicide combinations.

| | | | Inhibition in Combination with CGI factors | | | |
|---|---|---|---|---|---|---|
| Fungicide | | | Pep105 | Pep301 | Pep302 | Pep340 |
| | FRAC | | (SEQ ID | (SEQ ID | (SEQ ID | (SEQ ID |
| Name | Group | MOA | NO: 2182) | NO: 2188) | NO: 5837) | NO: 2240) |
| Fenpiclonil | FRAC 12 | MAPK osmotic | No | Yes | Yes | Yes |
| | | signal | | | | |
| | | transduction | | | | |
| Fludioxonil | FRAC 12 | MAPK osmotic | Not yet | No | Not yet | Yes |
| | | signal | tested | | tested | |
| | | transduction | | | | |

### Example 8

This example describes testing combinations of various CGI peptides and fungal signal transduction-inhibiting fungicides (e.g., phenylpyrrole fungicides such as fludioxonil or fenpiclonil) for their ability to decrease the growth or reproduction of the cucurbit downy mildew fungus using a leaf disc assay.

Pseudoperonospora cubensis (Cucurbit Downy Mildew) Assay Protocol

The assay is conducted as described in Example 1.

The treatments described in Example 7 are tested.

### Example 9

This example describes testing combinations of various CGI peptides and fungicides for their ability to decrease the growth or reproduction of a fungus.

### Reagents, Fungal Strains, and Growth Conditions.

Lyophilized peptides were synthesized by Genscript and stored at −20° C. until stocks were prepared. These CGI factor stocks were prepared in MilliQ water at a final concentration of 1 mM. Stocks were aliquoted into cryostorage tubes and stored at −80° C. until use in the assay.

Boscalid and Trifloxystrobin were purchased Sigma, USA. Stocks were prepared in 100% DMSO (dimethyl sulfoxide) at a final concentration of 1 mg/mL. Stocks were stored at room temperature until used in the assay.

F. proliferatum isolated from corn seeds was used for all assays. Briefly, corn seeds were soaked in water for at least an hour. The seeds were removed from the water and frozen at −20° C. overnight. They were then placed on plates of Fusarium-selective Nash and Snyder medium and incubated at room temperature. Once cultures had grown, a putative F. proliferatum isolate was chosen based on morphology and propagated on potato dextrose agar (PDA) (Difco, USA) at room temperature. DNA extraction and sequencing was then performed to confirm the isolate to be F. proliferatum.

Conidial stocks of F. proliferatum were prepared from 5 plates of 11-day-old cultures growing on PDA at room temperature. In a biosafety cabinet (BSC) about 3 mL of 0.01% Tween20 was added to each plate. A cell scraper was used to dislodge the conidia into suspension. The resulting liquid from each plate was passed through a 40-micron PluriSelect filter into a sterile 50 mL Falcon tube. Additionally, 0.01% Tween20 and 100% glycerol was added to the solution for a final volume of 20 mL at 20% glycerol. The concentration was quantified using a hemocytometer and was determined to be 1.39*10∂mg/mL. This solution was aliquoted into cryostorage tubes and stored at −80° C. until used in the assay.

The co-incubation assay was performed in 10% sucrose media. This was prepared by mixing 50 g of sucrose in 500 mL sterile water and filter sterilizing the resulting solution through a 0.2-micron filter. The media was stored at room temperature until used in the assay.

Co-Incubation (oCelloScope) Assay.

Fungal inhibition assays were performed using the oCelloScope (Biosense Solutions, Denmark) to determine the concentration at which CGI factors, Boscalid, and Trifloxystrobin demonstrated approximately 100%, 50%, and 0% inhibition of Fusarium proliferatum alone. The concentrations at which these phenotypes were observed are referred to as the treatment's high, medium, and low concentrations (H, M, and L, respectively). In these experiments, a treatment's relative minimum inhibitory concentration (MIC) is its high concentration. The same assay was then used to test CGI factors and each fungicide in various combination types to show a CGI factor's ability to reduce the MIC of fungal respiration inhibitors.

Briefly, F. proliferatum conidia at a final concentration of 10,000 conidia/mL were co-incubated with CGI factor, Boscalid, and Trifloxystrobin solo or combination treatments in 10% sucrose liquid media to a final volume of 150 ul in 4 separate wells of a 96 well plate and incubated for 24 hours at 23° C. Images of each well was captured using the oCelloScope, and those images were visually reviewed for fungal growth and conidial germination to determine which conditions demonstrated inhibition of F. proliferatum.

To conduct the oCelloScope assay, all solutions including 10% sucrose media, F. proliferatum conidia stock, fungicide stocks, and CGI factor stocks were brought to room temperature. Working stocks were prepared by performing dilutions of the original stocks in 10% sucrose. All conditions were prepared in individual microcentrifuge tubes by combining working stocks with media and conidia to achieve the final desired concentrations in a volume of 150 uL per replicate, with at least four replicates per condition. The solutions for each condition were then aliquoted, (150 uL in each well) into a sterile 96-well plate. Plates were incubated at 23° C. for 24 hours. An end point image was taken using the oCelloScope. These images were visually evaluated to determine conditions that demonstrated inhibition of F. proliferatum.

### Results

### Effect of Fusing a Cell Penetrating Peptide on Activity of CGI Factors

To determine whether a cell penetrating peptide could enhance the activity of antifungal peptides, one CGI factor was tested for activity against F. proliferatum with and without the cell penetrating peptide (CPP) octaarginine. To elucidate any benefit to fusing a CGI factor with a CPP, Pep340 (CGI octaarginine fusion, SEQ ID NO: 2240) and Pep462 (octaarginine, SEQ ID NO: 5747) were tested individually at different concentrations to compare range of activity. Both Pep340 and Pep462 were effective at 1 uM while only Pep340 showed activity at 0.5 uM (Table 17). This result suggests that fusing a CPP such as octaarginine to a CGI factor improves the efficacy of the peptide.

Boscalid and Trifloxystrobin in Combination with Different CGI Factors is Effective at Inhibiting Conidial Germination

To test whether CGI factors can be effective at controlling the growth and germination of F. proliferatum in combination with fungal respiration-inhibiting fungicides, we paired the CGI factors with boscalid and trifloxystrobin, representatives of this MOA. When tested by itself, neither boscalid nor trifloxystrobin showed any inhibition of conidial germination at their low and medium concentrations (0.2 ug/ml and 2 μg/mL, respectively) and wells treated with these compositions were characterized by profuse mycelial growth after 24 hr of incubation similar to the untreated condition. The CGI factors also did not exhibit any reduction in fungal growth at the low and medium concentrations when tested individually (Table 18). Interestingly, when the low concentration of trifloxystrobin was combined with either low or medium concentration of Pep301 (SEQ ID NO: 2188), no fungal growth or germination of conidia was observed. The same effect was observed when low concentration of trifloxystrobin was combined with a low concentration of PEP340 (SEQ ID NO: 2240) (Table 18). Boscalid showed no inhibition of fungal growth or conidial germination when tested on its own at 2 ug/mL, but the combination of boscalid with Pep301 at the peptide's medium and low concentrations (which had also shown no antifungal activity at these concentration) effectively inhibited conidial germination. These results summarized in Table 19 indicate that CGI factors and these fungal respiration-inhibiting fungicides can work together to inhibit conidial germination and therefore be a potential solution for management of fungal diseases. In addition, a combination of peptides with fungal respiration-inhibiting fungicides can reduce the concentration of applied fungal respiration-inhibiting fungicide in the field and provide a solution for resistance management for F. proliferatum.

TABLE 17
Inhibition of conidial germination by PEP 340 (CGI-octaarginine
fusion, SEQ ID NO: 2240) and PEP 462 (octaarginine alone,
SEQ ID NO: 5747) at different concentrations.

Inhibition of conidial germination

| Peptide | 1 uM | 0.5 uM | 0.25 uM | 0.125 uM |
|---|---|---|---|---|
| Pep340 | Yes | Yes | No | No |
| Pep462 | Yes | No | No | No |

Boscalid and Trifloxystrobin were tested in combination with PEP340 (SEQ ID NO: 2240), PEP301 (SEQ ID NO: 2188), and PEP302 (SEQ ID NO: 5837) at different concentrations to test cumulative effects on conidial germination of F. proliferatum. Shown in Table 18 are the concentrations of each fungal respiration-inhibiting fungicide and CGI factor tested either alone or in combination along with the effect of the treatment on inhibition of conidial germination observed after 24 hours at 23° C. Table 19 is a results table summarizing observations of growth inhibition of Boscalid and Trifloxystrobin with different CGI factors

TABLE 18
Effect of fungal respiration-inhibiting fungicides and CGI
combinations on F. proliferatum conidial germination.

| | | Fungicide | | Peptide | |
|---|---|---|---|---|---|
| Treatment | Fungicide | (ug/ml) | Peptide | (uM) | Inhibition |
| Untreated | — | — | — | — | No |
| Boscalid | Boscalid | 2 | — | — | No |
| Medium | | | | | |
| Boscalid | Boscalid | 0.2 | — | — | No |
| Low | | | | | |
| Trifloxytrobin | Trifloxystrobin | 2 | — | — | No |
| Medium | | | | | |
| Trifloxytrobin | Trifloxystrobin | 0.2 | — | — | No |
| Low | | | | | |
| Pep301 Low | — | — | Pep301 | 0.25 | No |
| Pep301 | — | — | Pep301 | 0.5 | No |
| Medium | | | | | |
| Pep301 High | — | — | Pep301 | 1.0 | Yes |
| Pep340 Low | — | — | Pep340 | 0.125 | No |
| Pep340 + | Trifloxystrobin | 2 ug/mL | Pep301 | 0.5 | Yes |
| Trifloxystrobin LM | | | | | |
| Pep340 + | Trifloxystrobin | 2 ug/mL | Pep301 | 0.25 | Yes |
| Trifloxystrobin LL | | | | | |
| Pep340 + | Trifloxystrobin | 0.2 ug/mL | Pep340 | 0.125 | Yes |
| Trifloxystrobin LL | | | | | |
| Pep301 + | Boscalid | 2 ug/mL | Pep301 | 0.5 | Yes |
| Boscalid MM | | | | | |
| Pep301 + | Boscalid | 2 ug/mL | Pep301 | 0.25 | Yes |
| Boscalid ML | | | | | |

TABLE 19
Summary of CGI with Boscalid and Trifloxystrobin combinations.

| | | | Inhibition in Combination with CGI factors | | | |
|---|---|---|---|---|---|---|
| Fungicide | | | Pep105 | Pep301 | Pep302 | Pep340 |
| | FRAC | | (SEQ ID | (SEQ ID | (SEQ ID | (SEQ ID |
| Name | Group | MOA | NO: 2182) | NO: 2188) | NO: 5837) | NO: 2240) |
| Boscalid | FRAC 7 | Succinate | Not | Yes | Not | Awaited |
| | | dehydrogenase | tested | | tested | |
| | | inhibitor | | | | |
| Trifloxystrobin | FRAC 11 | QoI-fungicide | Not | Yes | Not | Yes |
| | | | tested | | tested | |

### Example 10

This example describes testing combinations of various CGI peptides and fungicides for their ability to decrease the growth or reproduction of the cucurbit downy mildew fungus using a leaf disc assay.

Pseudoperonospora cubensis (Cucurbit Downy Mildew) Assay Protocol

The assay is conducted as described in Example 1.

The treatments described in Example 9 are tested.

### Example 11

This example describes testing combinations of various CGI peptides and fungal amino acid and/or protein synthesis-inhibiting fungicides (e.g., anilinopyrimidine fungicides such as Cyprodinil) for their ability to decrease the growth or reproduction of a fungus.

### Reagents, Fungal Strains, and Growth Conditions

The CGI factors described in Example 1 are tested. Lyophilized peptides are produced by Genscript and stored at −20° C. until stocks are created. These CGI factor stocks are prepared in MilliQ water at a final concentration of 1 mM. Stocks are aliquoted into cryostorage tubes and stored at −80′C until use in the assay.

The anilinopyrimidine fungicide Cyprodinil is tested. Stocks are stored at room temperature until used in the assay.

To isolate F. proliferatum, corn seeds are soaked in water for at least an hour. The seeds are removed from the water and frozen at −20′C overnight. They are then placed on plates of Fusarium-selective Nash and Snyder medium. Plates are incubated at room temperature. Once cultures have grown, a putative Fusarium isolate is chosen based on morphology and propagated further on 1× potato dextrose agar (PDA) at room temperature. DNA extraction and sequencing are then performed to confirm the isolate as F. proliferatum.

Conidial stocks of F. proliferatum are prepared from 5 plates of 11-day-old cultures growing on PDA at room temperature. In a biosafety cabinet (BSC) about 3 mL of 0.01% Tween20 are added to each plate. A cell scraper is used to dislodge the conidia into suspension. The resulting liquid from each plate is passed through a 40-micron PluriSelect filter into a sterile 50 mL Falcon tube. Additionally, 0.01% Tween20 and 100% glycerol is added to the solution for a final volume of 20 mL at 20% glycerol. The concentration is quantified using a hemocytometer, determined to be 1.39*10∂mg/mL. This solution is aliquoted into cryostorage tubes and stored at −80° C. until use in the assay.

The media used in this assay is 10% sucrose. This is prepared by combining 50 g of sucrose with 500 mL sterile water, mixing well, then passing through a 0.2-micron filter. Media is stored at room temperature until used in the assay.

Co-Incubation (oCelloScope) Assay

Fungal inhibition assays are performed using the oCelloScope (Biosense Solutions) to determine the concentration at which CGI factors and Cyprodinil demonstrate approximately 100%, 50%, and 0% inhibition of Fusarium proliferatum alone. The concentrations at which these phenotypes are observed are referred to as the treatments high (“H”, 100% inhibition), medium (“M”, 50% inhibition), and low concentrations (“L”, 0% inhibition). In these experiments, a treatment's relative minimum inhibitory concentration (MIC) is its high concentration. The same assay is then used to test CGI factors and Cyprodinil in various combination types to show a CGI factor's ability to reduce the MIC of Cyprodinil and other fungicides of the same anilinopyrimidine class. To elucidate advantageous combinations, three types of combinations at sub-MIC (medium and low concentrations) are tested. For the first combination type, CGI factor and Cyprodinil are each tested together at their lowest concentrations—this condition is referred to as “low-low (LL)”. The second combination type pairs Cyprodinil's lowest concentration with the CGI factor's medium concentration—“fungicide low, CGI factor medium (LM)”. Finally, the third combination type pairs Cyprodinil's medium concentration with the CGI factor's lowest concentration—“fungicide medium, CGI factor low (ML)”.

Briefly, F. proliferatum conidia at a final concentration of 10,000 conidia/mL are co-incubated with CGI factor and Cyprodinil solo or combination treatments in 10% sucrose liquid media within a 96 well plate for about 24 hours at 23° C. Images of each well were taken using the oCelloScope, and those images were visually reviewed to determine which conditions demonstrated inhibition of F. proliferatum.

To conduct the oCelloScope assay, materials are first gathered and brought to room temperature. This includes media, F. proliferatum conidia stock, anilinopyrimidine fungicide stocks, CGI factor stocks, and sterile 96 well plate(s). Generally working stocks are prepared by performing dilutions of original stocks in 10% sucrose. All conditions are prepared in individual microcentrifuge tubes—combining working stocks with media and conidia to achieve final desired concentrations in a volume provisioning for 150 uL per replicate, at least four replicates per condition, plus 100 uL extra volume. The solutions for each condition are then aliquoted, 150 uL in each well, into a 96 well plate according to a predetermined plate map. Plates are incubated at 23° C. for at least 24 hours. An end point image is taken using the oCelloScope. These images are visually evaluated to determine which conditions demonstrate inhibition against F. proliferatum.

### Results

Through the oCelloScope assay, a combination is deemed successful if the observed inhibition resulting from the pair is greater than an additive effect, i.e., a combination produces inhibition while the components tested individually do not.

### Example 12

This example describes testing combinations of various CGI peptides and fungal amino acid and/or protein synthesis-inhibiting fungicides (e.g., anilinopyrimidine fungicides such as Cyprodinil) for their ability to decrease the growth or reproduction of the cucurbit downy mildew fungus using a leaf disc assay.

Pseudoperonospora cubensis (Cucurbit Downy Mildew) Assay Protocol

The assay is conducted as described in Example 1.

The treatments described in Example 11 are tested.

### Example 13: Antifungal Efficacy of Fungicides in Combination with Pep340 Towards Botrytis

This example tests the antifungal activity of the polypeptide PEP340, an alpha pheromone (alpha factor) fused at its C terminus to a cell-penetrating peptide (in this case, a polyarginine), having the sequence WHWLQLKPGQPMYRRRRRRRR (SEQ ID NO: 2240 or SEQ ID NO: 5752), in combination with drosomycin, caspofungin, or cyprodinil.

### Botrytis Inhibition Assay Protocol

### a) Peptide Synthesis

Peptides were synthesized using the solid-phase peptide synthesis method and resuspended in water to 1 mM. The stock solution was aliquoted in cryovials and stored at −80° C.

### b) Preparation of Botrytis Conidia Suspension

The Botrytis culture (Strain B05.10; Westerdijk Fungal Biodiversity Institute, Netherlands) was propagated on 0.5% V8 agar plates (200 mL V8 Juice, 3 g CaCO₃,15 g agar, 800 mL millipore water) at 20° C. in 12 h photoperiod for 21 days. Fungal conidia were then harvested by scraping a Botrytis plate covered with 1 mL water containing 0.1% Tween 80. Conidia were collected in a 50 mL falcon tube by passing the solution through a 40-micron filter. The solution was centrifuged at 6,000 rpm and the pellet was resuspended in 1 mL of 20% glycerol. The conidia concentration of the resulting solution was counted using a hemocytometer. The stock was diluted to 107 spores/mL using 20% glycerol and frozen in 100 microliters aliquots in −80° C. until further use.

### c) Antifungal Susceptibility Testing

Peptides were tested for activity against Botrytis, using the resazurin assay. F or this assay, fungal stock conidia were diluted to 5,000 conidia in 1×PDB medium. The spore suspension was vortexed thoroughly. An aliquot of 50 microliters of the spore suspension was added to each well of a sterile 96-well clear bottom testing plate. Peptide (Pep340 or drosomycin) stocks of 1 mM were diluted to 2× and 4× of the desired end concentration and mixed either alone or in combination with fungal conidia to achieve a final volume of 100 microliters per well. For testing the peptides/fungicide combinations, the fungicide likewise was prepared at a 2× and 4× concentrations for testing alone or in combination with PEP340, respectively. The positive control, Natamycin, was prepared by diluting 10 mM stock solution (in DMSO) to a final concentration of 100 μM/well using sterile distilled water.

The plate was incubated for 24 h at 20° C. After 24 h, 10 microliters of resazurin dye were added to the microtiter plate, which was then incubated for 20 h. Fluorescence was measured at 570/620 nm after 22 h of incubation with resazurin on the Synergy X HI plate reader (BioTeK), with a read height of 7 mm. Growth inhibition was calculated by subtracting the fluorescence values of conidia only wells from sample wells and depicted as % inhibition of fungal growth. Four technical replicates were used for all treatments.

All agents that showed greater than 90% inhibition were considered to be active against Botrytis. Any treatment showing >90% inhibition in the assay was considered as inhibiting fungal growth. Any peptide, or combination of peptides or peptides/fungicides showing less than 90% inhibition were considered inactive or not consistently active.

### Results

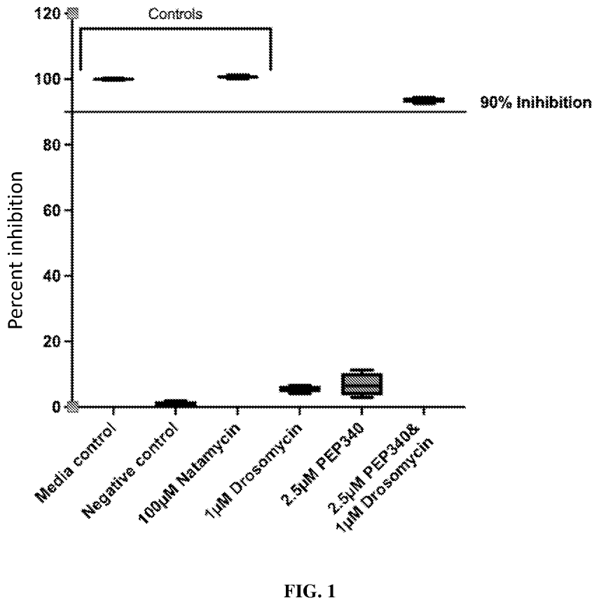

The inhibition assay results show that the combination of drosomycin with alpha factor fusion (PEP340) inhibited Botrytis growth (>90% inhibition) (Table 20), while each of the peptides alone had no effect on fungal growth by themselves at the tested concentrations (Table 21 and FIG. 1). At least in this group of experiments, the addition or combination of PEP340 with caspofungin (three experiments) or with cyprodinil (two experiments) did not consistently result in an improvement of antifungal activity of the non-CGI fungicide (i.e., caspofungin or cyprodinil), as summarized in Table 21.

TABLE 20
Conidial germination inhibition by the combination
of drosomycin with alpha pheromone-CPP fusion polypeptide
PEP340 (SEQ ID NO: 2240 or SEQ ID NO: 5752)

| | Percent | Standard |
|---|---|---|
| Treatment | Inhibition (Median) | Deviation |
| Media control | 99.98% | 0.16 |
| Negative control | −0.50% | 1.55 |
| 100 μM Natamycin | 100.65% | 0.26 |
| control | | |
| 1 μM Drosomycin + | 93.86% | 0.71 |
| 2.5 μM PEP340 | | |
| 1 μM Drosomycin | 5.86% | 0.92 |
| 2.5 μM PEP340 | 6.52% | 3.04 |

TABLE 21
Conidial germination inhibition by the combination of alpha pheromone-
CPP fusion polypeptide PEP340 (SEQ ID NO: 2240 or SEQ ID NO:
5752) with caspofungin (A, B, C) or cyprodinil (D, E).
A: EXP1: Caspofungin combination

| | Percent | |
|---|---|---|
| | Inhibition | Standard |
| Condition Name | EXP23002534 | Deviation |
| Media control | 99.96% | 0.21 |
| Negative control | 0.21% | 1.03 |
| 100 μM Natamycin | 100.48% | 0.19 |
| (positive control 1) | | |
| 1.95 nM Casp + | 1.48% | 1.23 |
| 2.5 μM PEP340 | | |
| 2.5 μM PEP340 | 7.54% | 2.50 |
| 1 μM Caspofungin | 1.08% | 1.26 |

B: EXP2: Caspofungin combination

| | Percent | |
|---|---|---|
| | Inhibition | Standard |
| Condition Name | EXP23002594 | Deviation |
| Media control | 99.95% | 0.23 |
| Negative control | −0.03% | 1.62 |
| 100 μM Natamycin | 100.55% | 0.22 |
| (positive control 1) | | |
| 1.95 nM Casp + | 98.85% | 0.26 |
| 2.5 μM PEP340 | | |
| 2.5 μM PEP340 | 9.26% | 3.46 |
| 1 μM Caspofungin | 98.73% | 1.14 |

C: EXP3: Caspofungin combination

| | Percent | |
|---|---|---|
| | Inhibition | Standard |
| Condition Name | EXP23002662 | Deviation |
| Media control | 100.06% | 0.21 |
| Negative control | 0.09% | 0.37 |
| 100 μM Natamycin | 100.64% | 0.28 |
| (positive control 1) | | |
| 1.95 nM Casp + | 38.99% | 22.62 |
| 2.5 μM PEP340 | | |
| 2.5 μM PEP340 | 5.3% | 1.74 |
| 1 μM Caspofungin | 3.1% | 0.79 |

D: EXP1: Cyprodinil combination

| | Percent | |
|---|---|---|
| | Inhibition | Standard |
| Condition Name | EXP23002594 | Deviation |
| Media control | 99.95% | 0.23 |
| Negative control | −0.03% | 1.62 |
| 100 μM Natamycin | 100.55% | 0.22 |
| (positive control 1) | | |
| 50 μM Cyp + | 81.72% | 2.25 |
| 2.5 μM PEP340 | | |
| 2.5 μM PEP340 | 9.26% | 3.46 |
| 50 μM Cyprodinil | 73.63% | 1.83 |
| 2% DMSO | −1.32% | 2.06 |

E: EXP2: Cyprodinil combination

| | Percent | |
|---|---|---|
| | Inhibition | Standard |
| Condition Name | EXP23002662 | Deviation |
| Media control | 100.06% | 0.21 |
| Negative control | 0.09% | 0.37 |
| 100 μM Natamycin | 100.64% | 0.28 |
| (positive control 1) | | |
| 50 μM Cyp + | 76.31% | 4.93 |
| 2.5 μM PEP340 | | |
| 2.5 μM PEP340 | 5.3% | 1.74 |
| 50 μM Cyprodinil | 53.74% | 3.67 |
| 100 μM Cyp + | 74.94% | 4.08 |
| 2.5 μM PEP340 | | |
| 100 μM Cyprodinil | 74.75% | 2.85 |
| 2% DMSO | −5.87% | 1.23 |

### Example 14

This example describes further embodiments of compositions including at least one CGI factor and one or more additional components, such as an herbicide, insecticide, nematicide, fungicide (other than the CGI factor, CGI factor precursor, CGI factor fragment, or CGI factor motifs herein disclosed), attractant, or bait.

Non-limiting embodiments of the one or more CGI factors (e.g., two or more copies of a CGI factor or multiple different CGI factors), CGI factor precursors, CGI factor fragments, or CGI factor motifs, include, for example, an amino acid sequence that has at least 80%, 85%, 90%, 95%, 97%, 98%, 99%, or 100% sequence identity with a sequence selected from the group consisting of SEQ ID NO: 961-1920, SEQ ID NO: 1921-2189, SEQ ID NOs: 2194-2210, SEQ ID NOs: 2215-2243, SEQ ID NOs: 2457-3361, SEQ ID NOs: 5707-5731, and SEQ ID NOs: 5755-5911; an alpha pheromone (e.g., a peptide with the sequence of SEQ ID NOs: 2182, 2183, or 2184), a homodimer of an alpha pheromone, with or without a neutral amino-acid linker, optionally wherein the homodimer is a peptide with a sequence selected from the group consisting of SEQ ID NO: 5837 and 2188, and SEQ ID NOs: 5835-5840, 5841-5845, and 5846-5911, or a polypeptide including at least one alpha pheromone (e.g., at least one peptide with a sequence of SEQ ID NOs: 2182, 2183, 2184, 2185, 2186, or 2187) fused to another peptide sequence such as a cell-penetrating peptide (CPP, e.g., a peptide with a sequence selected from the group consisting of SEQ ID NOs: 5736-5749); examples of alpha pheromone-CPP fusion peptides include the peptides with the sequence of SEQ ID NOs: 2240, 5752, 5753, 5750, 5751, 5754, 5755, 5756, 5757, 5758, 5759, 5760, or 5761. Any one or more of these CGI factors can be combined into a composition having one or more additional components, such as an herbicide, insecticide, nematicide, fungicide (other than the CGI factor, CGI factor precursor, CGI factor fragment, or CGI factor motifs herein disclosed), attractant, bait, antibacterial, antiviral, or other therapeutic, and such compositions can be appropriately formulated, e.g., for agricultural, pharmaceutical, or veterinary purposes, or for treating structures, materials, or artifacts to be protected from fungal damage. Examples of these additional components are provided in the following paragraphs.

Examples of pesticidal agents such as insecticides include representatives of the following classes: (1) Acetylcholinesterase (AChE) inhibitors which are carbamates (e.g., alanycarb, aldicarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, triazamate, trimethacarb, XMC, and xylylcarb) or organophosphates (e.g., acephate, azamethiphos, azinphos-ethyl, azinphos-methyl, cadusafos, chlorethoxyfos, chlorfenvinphos, chlormephos, chlorpyrifos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothiophosphoryl)salicylate, isoxathion, malathion, mecarbam, methamidophos, methidathion, mevinphos, monocrotophos, naled, omethoate, oxydemeton-methyl, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, triclorfon, and vamidothion); (2) GABA-gated chloride channel blockers which are cyclodiene-organochlorines (e.g., chlordane and endosulfan) or phenylpyrazoles (fiproles) (e.g., ethiprole and fipronil); (3) Sodium channel modulators which are pyrethroids (e.g., acrinathrin, allethrin, d-cis-trans allethrin, d-trans allethrin, bifenthrin, bioallethrin, bioallethrin s-cyclopentenyl isomer, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin [(lR)-trans-isomer], deltamethrin, empenthrin [(EZ)-(lR)-isomer], esfenvalerate, etofenprox, fenpropathrin, fenvalerate, flucythrinate, flumethrin, tau-fluvalinate, halfenprox, imiprothrin, kadethrin, momfluorothrin, permethrin, phenothrin [(lR)-trans-isomer], prallethrin, pyrethrins (pyrethrum), resmethrin, silafluofen, tefluthrin, tetramethrin, tetramethrin [(1R)-isomer)], tralomethrin, transfluthrin, DDT and methoxychlor); (4) Nicotinic acetylcholine receptor (nAChR) competitive modulators which are neonicotinoids (e.g., acetamiprid, clothianidin, dinotefuran, imidacloprid, nitenpyram, thiacloprid and thiamethoxam), or nicotine, or sulfoximines (e.g., sulfoxaflor), or butenolids (e.g., flupyradifurone), or mesoionics (e.g., triflumezopyrim); (5) Nicotinic acetylcholine receptor (nAChR) allosteric modulators which are spinosyns (e.g., spinetoram and spinosad); (6) Glutamate-gated chloride channel (GluCl) allosteric modulators which are avermectins/milbemycins (e.g., abamectin, emamectin benzoate, lepimectin, and milbemectin); (7) Juvenile hormone mimics which are juvenile hormone analogues (e.g., hydroprene, kinoprene, methoprene, fenoxycarb and pyriproxyfen); (8) Miscellaneous non-specific (multi-site) inhibitors which are alkyl halides (e.g., methyl bromide and other alkyl halides), or chloropicrine, or sulphuryl fluoride, or borax, or tartar emetic, or methyl isocyanate generators (e.g., diazomet and metam); (9) Chordotonal organ TRPV channel modulators (e.g., pymetrozine and pyrifluquinazone); (10) Mite growth inhibitors (e.g., clofentezine, hexythiazox, diflovidazin, and etoxazole); (11) Microbial disruptors of the insect gut membrane (e.g., Bacillus thuringiensis subspecies israelensis, Bacillus sphaericus, Bacillus thuringiensis subspecies aizawai, Bacillus thuringiensis subspecies kurstaki, and Bacillus thuringiensis subspecies tenebrionis) and pesticidally active proteins (e.g., Bt proteins originating from Bacillus thuringiensis); (12) Inhibitors of mitochondrial ATP synthase which are ATP disruptors (e.g., diafenthiuron), or organotin compounds (e.g., azocyclotin, cyhexatin, and fenbutatin oxide), or propargite, or tetradifon; (13) Uncouplers of oxidative phosphorylation via disruption of the proton gradient (e.g., chlorfenapyr, DNOC, and sulfluramid); (14) Nicotinic acetylcholine receptor channel blockers (e.g., bensultap, cartap hydrochloride, thiocylam, and thiosultap-sodium); (15) Inhibitors of chitin biosynthesis, type 0 (e.g., bistrifluron, chlorfluazuron, diflubenzuron, flucycloxuron, flufenoxuron, hexaflumuron, lufenuron, novaluron, noviflumuron, teflubenzuron, and triflumuron); (16) Inhibitors of chitin biosynthesis, type 1 (e.g., buprofezin); (17) Moulting disrupter, particularly for dipterans (e.g., cyromazine); (18) Ecdysone receptor agonists (e.g., chromafenozide, halofenozide, methoxyfenozide, and tebufenozide); (19) Octopamine receptor agonists (e.g., amitraz); (20) Mitochondrial complex III electron transport inhibitors (e.g., hydramethylnone, acequinocyl, and fluacrypyrim); (21) Mitochondrial complex I electron transport inhibitors which are METI acaricides (e.g., fenazaquin, fenpyroximate, pyrimidifen, pyridaben, tebufenpyrad, and tolfenpyrad) or rotenone (Derris); (22) Voltage-dependent sodium channel blockers (e.g., indoxacarb and metaflumizone); (23) Inhibitors of acetyl CoA carboxylase which are tetronic and tetramic acid derivatives (e.g., spirodiclofen, spiromesifen, and spirotetramat); (24) Mitochondrial complex IV electron transport inhibitors which are phosphines (e.g., aluminium phosphide, calcium phosphide, phosphine, and zinc phosphide) or cyanides (e.g., calcium cyanide, potassium cyanide and sodium cyanide); (25) Mitochondrial complex II electron transport inhibitors which are beta-ketonitrile derivatives (e.g., cyenopyrafen and cyflumetofen) or carboxanilides (e.g., pyflubumide); (26) Ryanodine receptor modulators which are diamides (e.g., chlorantraniliprole, cyantraniliprole, and flubendiamide); and (27) Chordotonal organ modulators (with undefined target site) (e.g., flonicamid). Also envisioned are agricultural formulations including one or more CGI factor, CGI factor precursor, CGI factor fragment, or CGI factor motifs and additionally including a pesticidal agent selected from the group consisting of acynonapyr, afidopyropen, afoxolaner, azadirachtin, benclothiaz, benzoximate, benzpyrimoxan, bifenazate, broflanilide, bromopropylate, chinomethionat, chloroprallethrin, cryolite, cyclaniliprole, cyclobutrifluram, cycloxaprid, cyhalodiamide, cyproflanilide, dicloromezotiaz, dicofol, dimpropyridaz, epsilon-metofluthrin, epsilon-momfluthrin, flometoquin, fluazaindolizine, fluensulfone, flufenerim, flufenoxystrobin, flufiprole, fluhexafon, flupentiofenox, fluopyram, flupyrimin, fluralaner, fluxametamide, fufenozide, guadipyr, heptafluthrin, imidaclothiz, iprodione, isocycloseram, kappa-bifenthrin, kappa-tefluthrin, lotilaner, meperfluthrin, oxazosulfyl, paichongding, pyridalyl, pyrifluquinazon, pyriminostrobin, spirobudiclofen, spiropidion, sulfur, tetramethylfluthrin, tetraniliprole, tetrachlorantraniliprole, tigolaner, tioxazafen, thiofluoximate, iodomethane; 1-{2-fluoro-4-methyl-5-[(2,2,2-trifluoroethyl)sulphinyl]phenyl}-3-(trifluoromethyl)-1H-1,2,4-triazole-5-amine, {1′-[(2E)-3-(4-chlorophenyl)prop-2-en-1-yl]-5-fluorospiro [indol-3,4′-piperidin]-1(2H)-yl}(2-chloropyridin-4-yl)methanone, 2-chloro-N-[2-{1-[(2E)-3-(4-chlorophenyl)prop-2-en-1-yl]piperidin-4-yl}-4-(trifluoromethyl)phenyl]isonicotinamide, 3-(4-chloro-2,6-dimethylphenyl)-4-hydroxy-8-methoxy-1,8-diazaspiro[4.5]dec-3-en-2-one, 3-(4-chloro-2,6-dimethylphenyl)-8-methoxy-2-oxo-1,8-diazaspiro[4.5]dec-3-en-4-yl ethyl carbonate, 4-(but-2-yn-1-yloxy)-6-(3,5-dimethylpiperidin-1-yl)-5-fluoropyrimidine, PF1364 (known from JP2010/018586), (3E)-3-[1-[(6-chloro-3-pyridyl)methyl]-2-pyridylidene]-1,1,1-trifluoro-propan-2-one, N-[3-(benzylcarbamoyl)-4-chlorophenyl]-1-methyl-3-(pentafluoroethyl)-4-(trifluoromethyl)-1H-pyrazole-5-carboxamide, 5-bromo-4-chloro-N-[4-chloro-2-methyl-6-(methylcarbamoyl)phenyl]-2-(3-chloro-2-pyridyl)pyrazole-3-carboxamide, 4-[5-(3,5-dichlorophenyl)-4,5-dihydro-5-(trifluoromethyl)-3-isoxazolyl]-2-methyl-N-(cis-1-oxido-3-thietanyl)-benzamide, 4-[5-(3,5-dichlorophenyl)-4,5-dihydro-5-(trifluoromethyl)-3-isoxazolyl]-2-methyl-N-(trans-1-oxido-3-thietanyl)-benzamide and 4-[(5S)-5-(3,5-dichlorophenyl)-4,5-dihydro-5-(trifluoromethyl)-3-isoxazolyl]-2-methyl-N-(cis-1-oxido-3-thietanyl)benzamide, N-[3-chloro-1-(3-pyridinyl)-1H-pyrazol-4-yl]-N-ethyl-3-[(3,3,3-trifluoropropyl)sulfinyll]-propanamide, (+)-N-[3-chloro-1-(3-pyridinyl)-1H-pyrazol-4-yl]-N-ethyl-3-[(3,3,3-trifluoropropyl)sulfinyl]-propanamide and (−)-N-[3-chloro-1-(3-pyridinyl)-1H-pyrazol-4-yl]-N-ethyl-3-[(3,3,3-trifluoropropyl)sulfinyl]-propanamide, 5-[[(2E)-3-chloro-2-propen-1-yl]amino]-1-[2,6-dichloro-4-(trifluoromethyl)phenyl]-4-[(trifluoromethyl)sulfinyl]-1H-pyrazole-3-carbonitrile, 3-bromo-N-[4-chloro-2-methyl-6-[(methylamino)thioxomethyl]phenyl]-1-(3-chloro-2-pyridinyl)-1H-pyrazole-5-carboxamide; N-[4-chloro-2-[[(1,1-dimethylethyl)amino]carbonyl]-6-methylphenyl]-1-(3-chloro-2-pyridinyl)-3-(fluoromethoxy)-1H-Pyrazole-5-carboxamide, N-[2-(5-amino-1,3,4-thiadiazol-2-yl)-4-chloro-6-methylphenyl]-3-bromo-1-(3-chloro-2-pyridinyl)-1H-pyrazole-5-carboxamide, 4-[3-[2,6-dichloro-4-[(3,3-dichloro-2-propen-1-yl)oxy]phenoxy]propoxy]-2-methoxy-6-(trifluoromethyl)-pyrimidine; (2E)- and 2(Z)-2-[2-(4-cyanophenyl)-1-[3-(trifluoromethyl)phenyl]ethylidene]-N-[4-(difluoromethoxy)phenyl]-hydrazinecarboxamide; 3-(2,2-dichloroethenyl)-2,2-dimethyl-4-(1H-benzimidazol-2-yl)phenyl-cyclopropanecarboxylic acid ester; (4aS)-7-chloro-2,5-dihydro-2-[[(methoxycarbonyl)[4-[(trifluoromethyl)thio]phenyl]amino]carbonyl]-indeno[1,2-e][1,3,4]oxadiazine-4a(3H)-carboxylic acid methyl ester; 6-deoxy-3-O-ethyl-2,4-di-O-methyl-1-[N-[4-[1-[4-(1,1,2,2,2-pentafluoroethoxy)phenyl]-1H-1,2,4-triazol-3-yl]phenyl]carbamate]-α-L-mannopyranose; 8-(2-cyclopropylmethoxy-4-trifluoromethyl-phenoxy)-3-(6-trifluoromethyl-pyridazin-3-yl)-3-aza-bicyclo[3.2.1]octane, (8-anti)-8-(2-cyclopropylmethoxy-4-trifluoromethyl-phenoxy)-3-(6-trifluoromethyl-pyridazin-3-yl)-3-aza-bicyclo[3.2.1]octane, (8-syn)-8-(2-cyclopropylmethoxy-4-trifluoromethyl-phenoxy)-3-(6-trifluoromethyl-pyridazin-3-yl)-3-aza-bicyclo[3.2.1]octane, N-[3-chloro-1-(3-pyridinyl)-1H-pyrazol-4-yl]-N-ethyl-3-[(3,3,3-trifluoropropyl)thio]-propanamide and N-[4-(aminothioxomethyl)-2-methyl-6-[(methylamino)carbonyl]phenyl]-3-bromo-1-(3-chloro-2-pyridinyl)-1H-pyrazole-5-carboxamide, 5-(1,3-dioxan-2-yl)-4-[[4-(trifluoromethyl)phenyl]methoxy]-pyrimidine, 3-(4-chloro-2,6-dimethylphenyl)-8-methoxy-1-methyl-,8-diazaspiro[4.5]decane-2,4-dione, 3-(4-chloro-2,6-dimethylphenyl)-8-methoxy-1-methyl-2-oxo-1,8-diazaspiro[4.5]dec-3-en-4-yl-carbonic acid ethyl ester, and 4-[(5S)-5-(3,5-Dichloro-4-fluorophenyl)-4,5-dihydro-5-(trifluoromethyl)-3-isoxazolyl]-N-[(4R)-2-ethyl-3-oxo-4-isoxazobdinyl]-2-methyl-benzamide, 2-({2-fluoro-4-methyl-5-[(R)-(2,2,2-trifluoroethyl)sulfinyl]phenyl}imino)-3-(2,2,2-trifluoroethyl)-1,3-thiazolodin-4-one,1,4-dimethyl-2-[2-(pyridin-3-yl)-2H-indazol-5-yl]-1,2,4-triazolidine-3,5-dione, a terpene blend including as active ingredients substantially pure α-terpinene, substantially pure p-cymene, and substantially pure limonene in a relative ratio of about 35-45:12-20:10-15, spirobudifen, tiorantraniliprole, trifluenfuronate, indazapyroxamet, fenmezoditiaz, fluchlordiniliprole, spidoxamat, nicofluprole, cyetpyrafen, and flupentiofenox.

Further envisioned are agricultural formulations including one or more CGI factor, CGI factor precursor, CGI factor fragment, or CGI factor motifs and additionally including at least one biological control agent. The term “biological control” refers to the control of a pest (e.g., an insect, acarid, or nematode) or pathogen (e.g., a fungus, oomycete, bacterium, or virus) by the use of an organism such as a microorganism or plant or by the use of a metabolite produced by such a microorganism or plant. In some cases, biological control is achieved by the use of naturally occurring compounds or their derivatives. Biological control agents include isolated, pure cultures of a microorganism having the desired bioactivity, supernatants or extracts of such cultures, and plant material or extracts of plant material. Examples of biological control agents include bacteria (e.g., the group consisting of Bacillus thuringiensis subsp. aizawai, in particular strain ABTS-1857 (SD-1372; e.g., XENTARI® from Valent BioSciences); Bacillus mycoides, isolate J. (e.g., BmJ from Certis USA LLC, a subsidiary of Mitsui & Co.); Bacillus sphaericus, in particular Serotype H5a5b strain 2362 (strain ABTS-1743) (e.g., VECTOLEX® from Valent BioSciences, US); Bacillus thuringiensis subsp. kurstaki strain BMP 123 from Becker Microbial Products, IL; Bacillus thuringiensis subsp. aizawai, in particular serotype H-7 (e.g., FLORBAC® WG from Valent BioSciences, US); Bacillus thuringiensis subsp. kurstaki strain HD-1 (e.g., DIPEL® ES from Valent BioSciences, US); Bacillus thuringiensis subsp. kurstaki strain BMP 123 by Becker Microbial Products, IL; Bacillus thuringiensis israelensis strain BMP 144 (e.g., AQUABAC® by Becker Microbial Products IL); Burkholderia spp., in particular Burkholderia rinojensis strain A396 (also known as Burkholderia rinojensis strain MBI 305) (Accession No. NRRL B-50319; WO 2011/106491 and WO 2013/032693; e.g., MBI-206 TGAI and ZELTO® from Marrone Bio Innovations); Chromobacterium subtsugae, in particular strain PRAA4-1T (MBI-203; e.g., GRANDEVO® from Marrone Bio Innovations); Paenibacillus popilliae (formerly Bacillus popilliae; e g. MILKY SPORE POWDER™ and MILKY SPORE GRANULAR™ from St. Gabriel Laboratories); Bacillus thuringiensis subsp. israelensis (serotype El-14) strain AM65-52 (Accession No. ATCC 1276) (e.g., VECTOBAC® by Valent BioSciences, US); Bacillus thuringiensis var. kurstaki strain EVB-113-19 (e.g., BIOPROTEC® from AEF Global); Bacillus thuringiensis subsp. tenebrionis strain NB 176 (SD-5428; e.g., NOVODOR® FC from BioFa DE); Bacillus thuringiensis var. japonensis strain Buibui; Bacillus thuringiensis subsp. kurstaki strain ABTS 351; Bacillus thuringiensis subsp. kurstaki strain PB 54; Bacillus thuringiensis subsp. kurstaki strain SA 11; Bacillus thuringiensis subsp. kurstaki strain SA 12; Bacillus thuringiensis subsp. kurstaki strain EG 2348; Bacillus thuringiensis var. colmeri (e.g., TIANBAOBTC by Changzhou Jianghai Chemical Factory); Bacillus thuringiensis subsp. aizawai strain GC-91; Serratia entomophila (e.g., INVADE® by Wrightson Seeds); Serratia marcescens, in particular strain SRM (Accession No. MTCC 8708); and Wolbachia pipientis ZAP strain (e.g., ZAP MALES® from MosquitoMate). Other examples of biological control agents include fungi (e.g., Muscodor albus, in particular strain QST 20799 (Accession No. NRRL 30547); Muscodor roseus in particular strain A3-5 (Accession No. NRRL 30548); Beauveria bassiana, in particular strain ATCC 74040 (e.g., Naturalis® from Intrachem Bio Italia); strain GHA (Accession No. ATCC74250; e.g., BotaniGuard Es and Mycotrol-0 from Laverlam International Corporation); strain ATP02 (Accession No. DSM 24665); strain PPRI 5339 (e.g., BroadBand™ from BASF); strain PPRI 7315, strain R444 (e.g., Bb-Protec from Andermatt Biocontrol), strains IL197, IL12, IL236, IL10, IL 131, IL116 (all referenced in laronski, 2007. Use of Entomopathogenic Fungi in Biological Pest Management, 2007: ISBN: 978-81-308-0192-6), strain Bv025 (see e.g., Garcia et al. 2006. Manejo Integrado de Plagas y Agroecologia (Costa Rica) No. 77); strain BaGPK; strain ICPE 279, strain CG 716 (e.g., BoveMax® from Novozymes); Hirsutella citriformis, Hirsutella thompsonii (e.g., Mycohit and ABTEC from Agro Bio-tech Research Centre, IN); Lecanici Ilium lecanii (formerly known as Verticillium lecanii), in particular conidia of strain KVO1 (e.g., Mycotal® and Vertalec® from Koppert/Arysta), strain DAOM198499 or strain DAOM216596; Lecanicillium muscarium (formerly Verticillium lecanii), in particular strain VE 6/CABI(=IMI) 268317/CBS102071/ARSEF5128 (e.g., Mycotal from Koppert); Metarhizium anisopliae var acridum, e.g., ARSEF324 from GreenGuard by Becker Underwood, US or isolate IMI 330189 (ARSEF7486; e.g., Green Muscle by Biological Control Products); Metarhizium brunneum, e.g., strain Cb 15 (e.g., ATTRACAP® from BIOCARE); Metarhizium anisopliae, e.g., strain ESALQ 1037 (e.g., from Metarril® SP Organic), strain E-9 (e.g., from Metarril® SP Organic), strain M206077, strain C4-B (NRRL 30905), strain ESC1, strain 15013-1 (NRRL 67073), strain 3213-1 (NRRL 67074), strain C20091, strain C20092, strain F52 (DSM3884/ATCC 90448; e.g., BIO 1020 by Bayer CropScience and also e.g., Met52 by Novozymes) or strain ICIPE 78; Metarhizium robertsii 23013-3 (NRRL 67075); Nomuraea rileyi; Paecilomyces fumosoroseus (new: Isaria fumosorosea), in particular strains Apopka 97 (available as PreFeRal from Certis, USA), Fe9901 (available as NoFly from Natural industries, USA), ARSEF 3581, ARSEF 3302, ARSEF 2679 (ARS Collection of Entomopathogenic Fungal Cultures, Ithaca, USA), IfBOl (China Center for Type Culture Collection CCTCC M2012400), ESALQ1296, ESALQ1364, ESALQ1409 (ESALQ: University of Sao Paulo (Piracicaba, SP, Brazil)), CG1228 (EMBRAPA Genetic Resources and Biotechnology (Brasilia, DF, Brazil)), KCH J2 (Dymarska et ah, 2017; PLoS one 12(10)): e0184885), HIB-19, HIB-23, HIB-29, HIB-30 (Gandarilla-Pacheco et ak, 2018; Rev Argent Microbiol 50: 81-89), CHE-CNRCB 304, EH-511/3 (Flores-Villegas et ak, 2016; Parasites & Vectors 2016 9:176 doi: 10.1186/s13071-016-1453-1), CHE-CNRCB 303, CHE-CNRCB 305, CHE-CNRCB 307 (Gallou et ak, 2016; fungal biology 120 (2016) 414-423), EH-506/3, EH-503/3, EH-520/3, PFCAM, MBP, PSMB1 (National Center for Biological Control, Mexico; Castellanos-Moguel et ak, 2013; Revista Mexicana De Micologia 38: 23-33, 2013), RCEF3304 (Meng et ak, 2015; Genet Mol Biol. 2015 July-September; 38(3): 381-389), PFO1-N10 (CCTCC No. M207088), CCM 8367 (Czech Collection of Microorganisms, Bmo), SFP-198 (Kim et ak, 2010; Wiley Online: DOI 10.1002/ps.2020), K3 (Y anagawa et ak, 2015; J Chem Ecok 2015; 41(12): 118-1126), CLO 55 (Ansari Ali et ak, 2011; PLoS One. 2011; 6(1): e16108. DOI: 10.1371/joumakpone.0016108), IfTSOl, HTSO2, HTSO7 (Dong et ak 2016/PLoS ONE 11(5): e0156087. doi: 10.1371/joumakpone.0156087), PI (Sun Agro Biotech Research Centre, India), If-02, If-2.3, If-03 (Farooq and Freed, 2016; DOI: 10.1016/j.bjm.2016.06.002), Ifir AsC (Meyer et ak, 2008; J. Invertebr. Pathol. 99:96-102. 10.1016/j jip.2008.03.007), PC-013 (DSMZ 26931), P43A, PCC (Carrillo-Perez et ak, 2012; DOI 10.1007/sl 1274-012-1184-1), Pf04, Pf59, Pfl09 (KimJun et ak, 2013; Mycobiology 2013 December; 41(4): 221-224), FG340 (Han et ak, 2014; DOI: 10.5941/MYCO.2014.42.4.385), Pfirl, Pfr8, Pfr9, PfrlO, Pfirl 1, Pfr12 (Angel-Sahaghn et ak, 2005; Journal of Insect Science), Ifr531 (Daniel and Wyss, 2009; DOI 10.1111/j.1439-0418.2009.01410.x), IF-1106 (Insect Ecology and Biocontrol Laboratory, Shanxi Agricultural University), 19602, 17284 (Hussain et ak 2016, DOI: 10.3390/ijms17091518), 103011 (U.S. Pat. No. 4,618,578), CNRCB1 (Centro Nacional de Referenda de Control Biologico (CNRCB), Colima, Mexico), SCAU-IFCFO1 (Nian et ak, 2015; DOI: 10.1002/ps.3977), PFO1-N4 (Engineering Research Center of Biological Control, SCAU, Guangzhou, P. R. China) Pfr-612 (Institute of Biotechnology (IB—FCB-UANL), Mexico), Pf-Tim, Pf-Tiz, Pf-Hal, Pf-Tic (Chan-Cupul et ak 2013, DOI: 10.5897/AJMR12.493); Aschersonia aleyrodis; Beauveria brongniartii (e.g., Beaupro from Andermatt Biocontrol AG); Conidiobolus obscurus; Entomophthora virulenta (e.g., Vektor from Ecomic); Lagenidium giganteum; Metarhizium flavoviride; Mucor haemelis (e.g., BioAvard from Indore Biotech Inputs & Research); Nosema locustae; Pandora delphacis; Sporothrix insectorum (e.g., Sporothrix Es from Biocerto, BR); and Zoophtora radicans. Other examples of biological control agents include viruses, e.g., Adoxophyes honmai nucleopolyhedrovirus (AdhoNPV), e.g., isolate ADN001; Agrotis ipsilon multiple nucleopolyhedrovirus (AgipNPV), e.g., isolate from Illinois; Anticarsia gemmatalis (Woolly pyrol moth) multiple nucleopolyhedrovirus (AgMNPV) (e.g., products Baculo-soja from Nova Era Biotecnologia Agricola; Baculovirus Nitral from Nitral Urbana; Coopervirus SC from COODETEC), e.g., isolate 2D; Autographa californica (Alfalfa Looper) multiple nucleopolyhedrovirus (AcMNPV) (e.g., product VPN-ULTRA from Agricola El Sol, Loopex from Andermatt Biocontrol, Lepigen from AgBiTech), e.g., isolate C6; Galleria mellonella multiple nucleopolyhedrovirus (GmMNPV); Plutella xylostella multiple nucleopolyhedrovirus, e.g., isolate CL3; Spodoptera exempta multiple nucleopolyhedrovirus (SpexNPV); Trichoplusia ni multiple nucleopolyhedrovirus (TnMNPV); Bombyx mori (silkworm) nucleopolyhedrovirus (BmNPV), e.g., isolate T3; (C3.10) Bombyx mandarina nucleopolyhedrovirus (BomaNPV), e.g., isolate SI; Buzura suppressaria nucleopolyhedrovirus (BuzuNPV), e.g., isolate 513; Choristoneura fumiferana DEF multiple nucleopolyhedrovirus (CfDefNPV); Choristoneura fumiferana multiple nucleopolyhedrovirus (CfMNPV), e.g., isolate from Ireland; Choristoneura rosaceana nucleopolyhedrovirus (ChroNPV); Ecotropis obliqua nucleopolyhedrovirus (EcobNPV), e.g., isolate A1; Epiphyas postvittana nucleopolyhedrovirus (EppoNPV); Heliocoverpa armigera (cotton bollworm) nucleopolyhedrovirus (Hear-NPV) (e.g., Vivus® MAX and Armigen from AgBiTech, Helicovex from Andermatt Biocontrol, Keyun HaNPV), such as isolate Cl (HearNPV-Cl), isolate NNG1 (HearNPV-NNG1), isolate G4 (HearNPV-G4; Helicoverpa zea single nucleopolyhedrovirus (HzSNPV) (e.g., Gemstar from Certis USA, Diplomata from Koppert); Lymantria dispar (gypsy moth) multiple nucleopolyhedrovirus (LdMNPV) (e.g., Lymantria dispar from Andermatt Biocontrol, Gypcheck developed by the US Forestry Service); Mamestra brassicae multiple nucleopolyhedrovirus (MbMNPV), e.g., isolate from Oxford; Mamestra configurata nucleopolyhedrovirus A (MacoNPV-A), e.g., isolate 90/2 or isolate 90/4; Mamestra configurata nucleopolyhedrovirus B (MacoNPV-B), e.g., isolate 96B; Orgyia pseudotsugata (Douglas-fir tussock moth) multiple nucleopolyhedrovirus (OpMNPV) (e.g., Virtuss); Spodoptera exigua (beet armyworm) multiple nucleopolyhedrovirus (SeMNPV) (e.g., Spexit from Andermatt Biocontrol, Spod-X LC from Certis USA, Keyun SeNPV), e.g., isolate from the US; Spodoptera frugiperda (fall armyworm) multiple nucleopolyhedrovirus (SfMNPV) (e.g., Fawligen from AgBiTech), e.g., isolate 3AP2 or isolate 6NR; Spodoptera littoralis (African cotton leafworm) nucleopolyhedrovirus (SpliNPV) (e.g., Littovir from Andermatt Biocontrol), e.g., isolate M2; Spodoptera litura (oriental leafworm moth) nucleopolyhedrovirus (SpltNPV) (e.g., Keyun SpltNPV), e.g., isolate G2; Thysanoplusia orichalcea nucleopolyhedrovirus (ThorNPV), e.g., isolate A28; Trichoplusia ni single nucleopolyhedrovirus (TnSNPV); (C3.30) Wiseana signata nucleopolyhedrovirus (WisiNPV); Adoxophyes orana (summer fruit tortrix) granulovirus (AdorGV) (e.g., Capex from Andermatt Biocontrol); Agrotis segetum nucleopolyhedrovirus A (AgseNPV); Anagrapha falcifera multiple nucleopolyhedrovirus (AnfaNPV); Antheraea pemyi nucleopolyhedrovirus (AnpeNPV); Chrysodeixis chalcites nucleopolyhedrovirus (ChchNPV); Clanis bilineata nucleopolyhedrovirus (ClbiNPV); Euproctis pseudoconspersa nucleopolyhedrovirus (EupsNPV); Hyphantria cunea nucleopolyhedrovirus (HycuNPV); Leucania separata nucleopolyhedrovirus (LeseNPV); Maruca vitrata nucleopolyhedrovirus (MaviNPV); Orgyia leucostigma nucleopolyhedrovirus (OrleNPV); Orgyia pseudotsugata single nucleopolyhedrovirus (OpSNPV); Panolis flammea nucleopolyhedrovirus (PaflNPV); Rachiplusia on multiple nucleopolyhedrovirus (RoMNPV); Erinnyis ello (homworm) GV (ErelGV), e.g., isolate VG010; Artogeia rapae granulovirus (ArGV); Pieris brassicae granulovirus (PbGV), e.g., isolate 384; Choristoneura fumiferana granulovirus (ChfuGV), e.g., isolate Bonaventure; Cryptophlebia leucotreta (false codling moth) granulovirus (CrleGV) (e.g., Cryptex from Andermatt Biocontrol), e.g., isolate CV3; Cydia pomonella (codling moth) granulovirus (CpGV) (e.g., Madex® products from Andermatt Biocontrol, Carpovirus Plus from AgroRoca SA), e.g., isolate M1; Harrisina brillians granulovirus (HabrGV), e.g., isolate M2; Helicoverpa armigera (cotton bollworm) granulovirus (HearGV); Lacanobia oleracea granulovirus (LaolGV), e.g., isolate SI; Phthorimaea operculella (tobacco leaf miner) granulovirus (PhopGV) (e.g., Tutavir from Andermatt Biocontrol, Matapol); Plodia interpunctella granulovirus (PiGV), e.g., isolate B3; Plutella xylostella granulovirus (PlxyGV) (e.g., Plutellavex® from Keyun), e.g., isolate Kl; Pseudalatia unipuncta granulovirus (PsunGV), e.g., Hawaiian isolate; Trichoplusia ni granulovirus (TnGV), e.g., isolate M10-5: Xestia c-nigrum granulovirus (XecnGV), e.g., isolate alpha4; Agrotis segetum granulovirus (AgseGV), e.g., isolate Xinjiang; Choristoneura occidentalis granulovirus (ChocGV); Spodoptera litura (oriental leafworm moth) granulovirus (SpliGV), e.g., isolate Kl; Neodiprion lecontei (red-headed pinesawfly) nucleopolyhedrovirus (NeleNPV) (e.g., Lecontvirus from SYLVAR); Neodiprion sertifer (Pine sawfly) nucleopolyhedrovirus (NeseNPV) (e.g., Neocheck-S developed by the US Forestry Service; Gilpinia hercyniae nucleopolyhedrovirus (GiheNPV), e.g., isolate i7; Neodiprion abietis (balsam-fir sawfly) nucleopolyhedrovirus (NeabNPV) (e.g., ABIETIV from SYLVAR); Culex nigripalpus nucleopolyhedrovirus (CuniNPV), e.g., isolate from Florida (1997); Aedes sollicitans nucleopolyhedrovirus (AesoNPV); Uranotaenia sapphrinia nucleopolyhedrovirus (UrsaNPV); Spodoptera albula (gray-streaked armywom moth) NPV (e.g., VPN-ULTRA from Agricola El Sol); Biston suppressaria (tea looper) NPV; Dendrolimus punctatus (Masson pine moth) CPV; Leucoma salicis (satin moth) NPV; Spodoptera frugiperda granulovirus (SfGV), e.g., isolate ARG; Spodoptera sunia nulear polyhedrosisvirus (e.g., VPN 82 from Agricola El Sol); Pieris rapae (small white) GV (PiraGV); Spodoptera exigua (beet armyworm) nucleopolyhedrovirus (SeNPV) (e.g., Keyun SeNPV) and Zucchini yellow mosaic virus. Biological control agents include nematicidally active biological control agents, which include bacteria (e.g., Bacillus subtilis, in particular strain QST713/AQ713 (having NRRL Accession No. B-21661; available as SERENADE® OPTI or SERENADE® ASO from Bayer CropScience LP, US); Bacillus pumilus, in particular strain QST2808 (having Accession No. NRRL No. B-30087); Bacillus firmus, in particular, strain CNMC 1-1582 (e.g., VOTIVO® from BASF SE); Bacillus amyloliquefaciens, in particular strain FZB42 (e.g., RHIZOVITAL® from ABiTEP, DE); Bacillus amyloliquefaciens strain PTA-4838 (AVEO EZ® from Valent/Sumitomo; VARNIMO® ST from LidoChem); Bacillus cereus, in particular spores of Bacillus cereus strain CNCM 1-1562 (cf. U.S. Pat. No. 6,406,690); Bacillus laterosporus (also known as Brevibacillus laterosporus; e.g., BIO-TODE® from Agro-Organics, ZA); Bacillus megaterium, strain YFM3.25 (e.g., BIOARC® from BioArc); Bacillus mojavensis, strain SRI1 (CECT-7666; by Probelte S. A); Bacillus nematocida B16 (CGMCC Accession No. 1128); a mixture of Bacillus licheniformis FMCHOOl and Bacillus subtilis FMCH002 (available as QUARTZO® (WG), PRESENCE® (WP) from FMC Corporation); Pasteuria nishizawae (e.g., OYACYST® LF/ST from Pasteuria Bioscience; CLARIVA® PN from Syngenta/ChemChina); Burkholderia rinojensis strain A396 (also known as Burkholderia rinojensis strain MBI 305) (Accession No. NRRL B-50319; WO 2011/106491 and WO 2013/032693; MAJESTENE® from Marrone Bio Innovations); Pasteuria penetrans; Pasteuria usgae (e.g., ECONEM™ from Pasteuria Bioscience); Streptomycete sp., such as Streptomyces lydicus strain WYEC108 (also known as Streptomyces lydicus strain WY CD 108US) (ACTINO-IRON® and ACTINOVATE® from Novozymes); Streptomyces saraceticus (e.g., CLANDA® from A & A Group (Agro Chemical Corp.); Bacillus thuringiensis strain CR-371 (Accession No. ATCC 55273); Bacillus cepacia (e.g., DENY® from Stine Microbial Products); Lysobacter enzymogenes, in particular strain C3 (cf. J Nematol. 2006 June, 38(2): 233-239 and Biological Control 2018 February, 117: 158-163)); and fungi (e.g., Muscodor albus, in particular strain QST 20799 (Accession No. NRRL 30547); Muscodor roseus, in particular strain A3-5 (Accession No. NRRL 30548); Purpureocillium lilacinum (previously known as Paecilomyces lilacinus), in particular P. lilacinum strain 251 (AGAL 89/030550; e.g., BioAct from Bayer CropScience Biologies GmbH), strain 580 (BIOSTAT® WP (ATCC No. 38740) by Laverlam), strain in the product BIO-NEMATON® (T. Stanes and Company Ltd.), strain in the product MYSIS® (Varsha Bioscience and Technology India Pvt Ltd.), strain in the product BIOICONEMA® (Nico Orgo Maures, India), strain in the product NEMAT® (Ballagro Agro Tecnologia Ltda, Brazil), and a strain in the product SPECTRUM PAE L® (Promotora Tecnica Industrial, S.A. DE C.V., Mexico); Trichoderma koningii; Harposporium anguillullae; Hirsutella minnesotensis; Monacrosporium cionopagum; Monacrosporium psychrophilum; Myrothecium verrucaria, in particular strain AARC-0255 (e.g., DiTera™ by Valent Biosciences); Paecilomyces variotii, strain Q-09 (e.g., Nemaquim® from Quimia, MX); Stagonospora phaseoli (e.g., from Syngenta); Trichoderma lignorum, in particular strain TL-0601 (e.g., Mycotric from Futureco Bioscience, ES); Fusarium solani, strain Fs5; Hirsutella rhossiliensis; Monacrosporium drechsleri; Monacrosporium gephyropagum; Nematoctonus geogenius; Nematoctonus leiosporus; Neocosmospora vasinfecta; Paraglomus sp, in particular Paraglomus brasilianum; Pochonia chlamydosporia (also known as Vercillium chlamydosporium), in particular var. catenulata (IMI SD 187; e.Fg. KlamiC from The National Center of Animal and Plant Health (CENSA), CU); Stagonospora heteroderae; Meristacrum asterospermum, and Duddingtonia flagrans).

Further envisioned are agricultural formulations including one or more CGI factor, CGI factor precursor, CGI factor fragment, or CGI factor motifs and additionally including at least a second antifungal or fungicidal agent (other than the CGI factor, CGI factor precursor, CGI factor fragment, or CGI factor motifs). Examples of suitable antifungal or fungicidal agents other than the CGI factor, CGI factor precursor, CGI factor fragment, or CGI factor motifs include: (1) Inhibitors of ergosterol biosynthesis, e.g., cyproconazole, difenoconazole, epoxiconazole, fenhexamid, fenpropidin, fenpropimorph, fenpyrazamine, fluquinconazole, flutriafol, imazalil, imazalil sulfate, ipconazole, metconazole, myclobutanil, paclobutrazol, (1.016) prochloraz, propiconazole, prothioconazole, pyrisoxazole, spiroxamine, tebuconazole, tetraconazole, triadimenol, tridemorph, triticonazole, (1R,2S,5S)-5-(4-chlorobenzyl)-2-(chloromethyl)-2-methyl-1-(1H-1,2,4-triazol-1-ylmethyl) cyclopentanol, (1S,2R,5R)-5-(4-chlorobenzyl)-2-(chloromethyl)-2-methyl-1-(1H-1,2,4-triazol-1-ylmethyl)cyclo-pentanol, (2R)-2-(1-chlorocyclopropyl)-4-[(1R)-2,2-dichlorocyclopropyl]-1-(1H-1,2,4-triazol-1-yl)butan-2-ol, (2R)-2-(1-chlorocyclopropyl)-4-[(1S)-2,2-dichlorocyclopropyl]-1-(1H-1,2,4-triazol-1-yl)butan-2-ol, (2R)-2-[4-(4-chlorophenoxy)-2-(trifluoromethyl)phenyl]-1-(1H-1,2,4-triazol-1-yl)propan-2-ol, (2S)-2-(1-chlorocyclopropyl)-4-[(1R)-2,2-dichlorocyclopropyl]-1-(1H-1,2,4-triazol-1-yl)butan-2-ol, (2S)-2-(1-chlorocyclopropyl)-4-[(1S)-2,2-dichlorocyclopropyl]-1-(1H-1,2,4-triazol-1-yl)butan-2-ol, (2S)-2-[4-(4-chlorophenoxy)-2-(trifluoromethyl)phenyl]-1-(1H-1,2,4-triazol-1-yl)propan-2-ol, (R)-[3-(4-chloro-2-fluorophenyl)-5-(2,4-difluorophenyl)-1,2-oxazol-4-yl](pyridin-3-yl)methanol, (S)-[3-(4-chloro-2-fluorophenyl)-5-(2,4-difluorophenyl)-1,2-oxazol-4-yl](pyridin-3-yl)methanol, [3-(4-chloro-2-fluorophenyl)-5-(2,4-difluorophenyl)-1,2-oxazol-4-yl](pyridin-3-yl)methanol, 1-({(2R,4S)-2-[2-chloro-4-(4-chlorophenoxy)phenyl]-4-methyl-1,3-dioxolan-2-yl}methyl)-1H-1,2,4-triazole, 1-({(2S,4S)-2-[2-chloro-4-(4-chlorophenoxy)phenyl]-4-methyl-1,3-dioxolan-2-yl}methyl)-1H-1,2,4-triazole, 1-{[3-(2-chlorophenyl)-2-(2,4-difluorophenyl)oxiran-2-yl]methyl}-1H-1,2,4-triazol-5-yl thiocyanate, 1-{[rel(2R,3R)-3-(2-chlorophenyl)-2-(2,4-difluorophenyl)oxiran-2-yl]methyl}-1H-1,2,4-triazol-5-yl thiocyanate, 1-{[rel(2R,3S)-3-(2-chlorophenyl)-2-(2,4-difluorophenyl)oxiran-2-yl]methyl}-1H-1,2,4-triazol-5-yl thiocyanate, 2-[(2R,4R,5R)-1-(2,4-dichlorophenyl)-5-hydroxy-2,6,6-trimethylheptan-4-yl]-2,4-dihydro-3H-1,2,4-triazole-3-thione, 2-[(2R,4R,5S)-1-(2,4-dichlorophenyl)-5-hydroxy-2,6,6-trimethylheptan-4-yl]-2,4-dihydro-3H-1,2,4-triazole-3-thione, 2-[(2R,4S,5R)-1-(2,4-dichlorophenyl)-5-hydroxy-2,6,6-trimethylheptan-4-yl]-2,4-dihydro-3H-1,2,4-triazole-3-thione, 2-[(2R,4S,5S)-1-(2,4-dichlorophenyl)-5-hydroxy-2,6,6-trimethylheptan-4-yl]-2,4-dihydro-3H-1,2,4-triazole-3-thione, 2-[(2S,4R,5R)-1-(2,4-dichlorophenyl)-5-hydroxy-2,6,6-trimethylheptan-4-yl]-2,4-dihydro-3H-1,2,4-triazole-3-thione, 2-[(2S,4R,5S)-1-(2,4-dichlorophenyl)-5-hydroxy-2,6,6-trimethylheptan-4-yl]-2,4-dihydro-3H-1,2,4-triazole-3-thione, 2-[(2S,4S,5R)-1-(2,4-dichlorophenyl)-5-hydroxy-2,6,6-trimethylheptan-4-yl]-2,4-dihydro-3H-1,2,4-triazole-3-thione, 2-[(2S,4S,5S)-1-(2,4-dichlorophenyl)-5-hydroxy-2,6,6-trimethylheptan-4-yl]-2,4-dihydro-3H-1,2,4-triazole-3-thione, 2-[1-(2,4-dichlorophenyl)-5-hydroxy-2,6,6-trimethylheptan-4-yl]-2,4-dihydro-3H-1,2,4-triazole-3-thione, 2-[2-chloro-4-(2,4-dichlorophenoxy)phenyl]-1-(1H-1,2,4-triazol-1-yl)propan-2-ol, 2-[2-chloro-4-(4-chlorophenoxy)phenyl]-1-(1H-1,2,4-triazol-1-yl)butan-2-ol, 2-[4-(4-chlorophenoxy)-2-(trifluoromethyl)phenyl]-1-(1H-1,2,4-triazol-1-yl)butan-2-ol, 2-[4-(4-chlorophenoxy)-2-(trifluoromethyl)phenyl]-1-(1H-1,2,4-triazol-1-yl)pentan-2-ol, mefentrifluconazole, 2-{[3-(2-chlorophenyl)-2-(2,4-difluorophenyl)oxiran-2-yl]methyl}-2,4-dihydro-3H-1,2,4-triazole-3-thione, 2-{[rel(2R,3R)-3-(2-chlorophenyl)-2-(2,4-difluorophenyl)oxiran-2-yl]methyl}-2,4-dihydro-3H-1,2,4-triazole-3-thione, 2-{[rel(2R,3S)-3-(2-chlorophenyl)-2-(2,4-difluorophenyl)oxiran-2-yl]methyl}-2,4-dihydro-3H-1,2,4-triazole-3-thione, 5-(4-chlorobenzyl)-2-(chloromethyl)-2-methyl-1-(1H-1,2,4-triazol-1-ylmethyl)cyclopentanol, 5-(allylsulfanyl)-1-{[3-(2-chlorophenyl)-2-(2,4-difluorophenyl)oxiran-2-yl]methyl}-1H-1,2,4-triazole, 5-(allylsulfanyl)-1-{[rel(2R,3R)-3-(2-chlorophenyl)-2-(2,4-difluorophenyl)oxiran-2-yl]methyl}-1H-1,2,4-triazole, 5-(allylsulfanyl)-1-{[rel(2R,3S)-3-(2-chlorophenyl)-2-(2,4-difluorophenyl)oxiran-2-yl]methyl}-1H-1,2,4-triazole, N′-(2,5-dimethyl-4-{[3-(1,1,2,2-tetrafluoroethoxy)phenyl]sulfanyl}phenyl)-N-ethyl-N-methylimidoformamide, N′-(2,5-dimethyl-4-{[3-(2,2,2-trifluoroethoxy)phenyl]sulfanyl}phenyl)N-ethyl-N-methylimidoforinamide, N′-(2,5-dimethyl-4-{[3-(2,2,3,3-tetrafluoropropoxy)phenyl]sulfanyl}phenyl)-N-ethyl-N-methylimidoformamide, N′-(2,5-dimethyl-4-{[3-(pentafhioroethoxy)phenyl]sulfanyl}phenyl)-N-ethyl-N-methylimidoformamide, N′-(2,5-dimethyl-4-[3-[(1,1,2,2-tetrafluoroethyl)sulfanyl]-phenoxy}phenyl)-N-ethyl-N-methylimidoformamide, N′-(2,5-dimethyl-4-{3-[(2,2,2-trifluoroethyl)sulfanyl]phenoxy}phenyl)-N-ethyl-N-methylimidoformamide, N′-(2,5-dimethyl-4-{3-[(2,2,3,3-tetrafluoropropyl)sulfanyl]phenoxy phenyl)-N-ethyl-N-methylimidoformamide, N′-(2,5-dimethyl-4-{3-[(pentafluoroethyl)sulfanyl]phenoxy}phenyl)-N-ethyl-N-methylimidoformamide, N′-(2,5-dimethyl-4-phenoxyphenyl)-N-ethyl-N-methylimidoformamide, N′-(4-{[3-(difluoromethoxy)phenyl]sulfanyl}-2,5-dimethylphenyl)-N-ethyl-N-methylimidoformamide, N′-(4-{3-[(difluoromethyl)sulfanyl]phenoxy}-2,5-dimethylphenyl)-N-ethyl-N-methylimidoformamide, N′-[5-bromo-6-(2,3-dihydro-1H-inden-2-yloxy)-2-methylpyridin-3-yl]-N-ethyl-N-methylimidoformamide, N′-{4-[(4,5-dichloro-1,3-thiazol-2-yl)oxy]-2,5-dimethylphenyl}-N-ethyl-N-methylimidoformamide, N′-{5-bromo-6-[(1R)-1-(3,5-difluorophenyl)ethoxy]-2-methylpyridin-3-yl}-N-ethyl-N-methylimidoformamide, N′-{5-bromo-6-[(1S)-1-(3,5-difluorophenyl)ethoxy]-2-methylpyridin-3-yl}-N-ethyl-N-methylimidoformamide, N′-{5-bromo-6-[(cis-4-isopropylcyclohexyl)oxy]-2-methylpyridin-3-yl}-N-ethyl-N-methylimidoformamide, N′-{5-bromo-6-[(trans-4-isopropylcyclohexyl)oxy]-2-methylpyridin-3-yl}-N-ethyl-N-methylimidoformamide, N′-{5-bromo-6-[1-(3,5-difluorophenyl)ethoxy]-2-methylpyridin-3-yl}-N-ethyl-N-methylimidoformamide, ipfentrifluconazole, 2-[4-(4-chlorophenoxy)-2-(trifluoromethyl)phenyl]-1-(1H-1,2,4-triazol-1-yl)propan-2-ol, 2-[6-(4-bromophenoxy)-2-(trifluoromethyl)-3-pyridyl]-1-(1,2,4-triazol 1-yl)propan-2-ol, 2-[6-(4-chlorophenoxy)-2-(trifluoromethyl)-3-pyridyl]-1-(1,2,4-triazol-1-yl)propan-2-ol, 3-[2-(1-chlorocyclopropyl)-3-(3-chloro-2-fluoro-phenyl)-2-hydroxy-propyl]imidazole-4-carbonitrile, 4-[[6-[rac-(2R)-2-(2,4-difluorophenyl)-1,1-difluoro-2-hydroxy-3-(5-thioxo-4H-1,2,4-triazol-1-yl)propyl]-3-pyridyl]oxy]benzonitrile, N-isopropyl-N′-[5-methoxy-2-methyl-4-(2,2,2-trifluoro-1-hydroxy-1-phenyl-ethyl)phenyl]-N-methylimidoformamide, N′-{5-bromo-2-methyl-6-[(1-propoxypropan-2-yl)oxy]pyridin-3-yl}-N-ethyl-N-methylimidoformamide, hexaconazole, penconazole, fenbuconazole, methyl 2-[2-chloro-4-(4-chlorophenoxy)phenyl]-2-hydroxy-3-(1,2,4-triazol-1-yl)propanoate, and 2-[2-chloro-4-(4-chlorophenoxy)phenyl]-2-hydroxy-3-(1,2,4-triazol-1-yl)propanoic acid; (2) Inhibitors of the respiratory chain at complex I or II, e.g., benzovindiflupyr, bixafen, boscalid, carboxin, fluopyram, flutolanil, fluxapyroxad, furametpyr, isofetamid, isopyrazam (anti-epimeric enantiomer 1R,4S,9S), isopyrazam (anti-epimeric enantiomer 1S,4R,9R), isopyrazam (anti-epimeric racemate 1RS,4SR,9SR), isopyrazam (mixture of syn-epimeric racemate 1RS,4SR,9RS and anti-epimeric racemate 1RS,4SR,9SR), isopyrazam (syn-epimeric enantiomer 1R,4S,9R), isopyrazam (syn-epimeric enantiomer 1S,4R,9S), isopyrazam (syn-epimeric racemate 1RS,4SR,9RS), penflufen, penthiopyrad, pydiflumetofen, pyraziflumid, sedaxane, 1,3-dimethyl-N-(1,1,3-trimethyl-2,3-dihydro-1H-inden-4-yl)-1H-pyrazole-4-carboxamide, 1,3-dimethyl-N-[(3R)-1,1,3-trimethyl-2,3-dihydro-1H-inden-4-yl]-1H-pyrazole-4-carboxamide, 1,3-dimethyl-N-[(3S)-1,1,3-trimethyl-2,3-dihydro-1H-inden-4-yl]-1H-pyrazole-4-carboxamide, 1-methyl-3-(trifluoromethyl)-N-[2′-(trifluoromethyl)biphenyl-2-yl]-1H-pyrazole-4-carboxamide, 2-fluoro-6-(trifluoromethyl)-N-(1,1,3-trimethyl-2,3-dihydro-1H-inden-4-yl)benzamide, 3-(difluoromethyl)-1-methyl-N-(1,1,3-trimethyl-2,3-dihydro-1H-inden-4-yl)-1H-pyrazole-4-carboxamide, inpyrfluxam, 3-(difluoromethyl)-1-methyl-N-[(3S)-1,1,3-trimethyl-2,3-dihydro-1H-inden-4-yl]-1H-pyrazole-4-carboxamide, fluindapyr, 3-(difluoromethyl)-N-[(3R)-7-fluoro-1,1,3-trimethyl-2,3-dihydro-1H-inden-4-yl]-1-methyl-1H-pyrazole-4-carboxamide, 3-(difluoromethyl)-N-[(3S)-7-fluoro-1,1,3-trimethyl-2,3-dihydro-1H-inden-4-yl]-1-methyl-1H-pyrazole-4-carboxamide, 5,8-difluoro-N-[2-(2-fluoro-4-{[4-(trifluoromethyl)pyridin-2-yl]oxy}phenyl)ethyl]quinazolin-4-amine, N-(2-cyclopentyl-5-fluorobenzyl)-N-cyclopropyl-3-(difluoromethyl)-5-fluoro-1-methyl-1H-pyrazole-4-carboxamide, N-(2-tert-butyl-5-methylbenzyl)-N-cyclopropyl-3-(difluoromethyl)-5-fluoro-1-methyl-1H-pyrazole-4-carboxamide, N-(2-tert-butylbenzyl)-N-cyclopropyl-3-(difluoromethyl)-5-fluoro-1-methyl-1H-pyrazole-4-carboxamide, N-(5-chloro-2-ethylbenzyl)-N-cyclopropyl-3-(difluoromethyl)-5-fluoro-1-methyl-1H-pyrazole-4-carboxamide, isoflucypram, N-[(1R,4S)-9-(dichloromethylene)-1,2,3,4-tetrahydro-1,4-methanonaphthalen-5-yl]-3-(difluoromethyl)-1-methyl-1H-pyrazole-4-carboxamide, N-[(1S,4R)-9-(dichloromethylene)-1,2,3,4-tetrahydro-1,4-methanonaphthalen-5-yl]-3-(difluoromethyl)-1-methyl-1H-pyrazole-4-carboxamide, N-[1-(2,4-dichlorophenyl)-1-methoxypropan-2-yl]-3-(difluoromethyl)-1-methyl-1H-pyrazole-4-carboxamide, N-[2-chloro-6-(trifluoromethyl)benzyl]-N-cyclopropyl-3-(difluoromethyl)-5-fluoro-1-methyl-1H-pyrazole-4-carboxamide, N-[3-chloro-2-fluoro-6-(trifluoromethyl)benzyl]-N-cyclopropyl-3-(difluoromethyl)-5-fluoro-1-methyl-1H-pyrazole-4-carboxamide, N-[5-chloro-2-(trifluoromethyl)benzyl]-N-cyclopropyl-3-(difluoromethyl)-5-fluoro-1-methyl-1H-pyrazole-4-carboxamide, N-cyclopropyl-3-(difluoromethyl)-5-fluoro-1-methyl-N-[5-methyl-2-(trifluoromethyl)benzyl]-1H-pyrazole-4-carboxamide, N-cyclopropyl-3-(difluoromethyl)-5-fluoro-N-(2-fluoro-6-isopropylbenzyl)-1-methyl-1H-pyrazole-4-carboxamide, N-cyclopropyl-3-(difluoromethyl)-5-fluoro-N-(2-isopropyl-5-methylbenzyl)-1-methyl-1H-pyrazole-4-carboxamide, N-cyclopropyl-3-(difluoromethyl)-5-fluoro-N-(2-isopropylbenzyl)-1-methyl-1H-pyrazole-4-carbothioamide, N-cyclopropyl-3-(difluoromethyl)-5-fluoro-N-(2-isopropylbenzyl)-1-methyl-1H-pyrazole-4-carboxamide, N-cyclopropyl-3-(difluoromethyl)-5-fluoro-N-(5-fluoro-2-isopropylbenzyl)-1-methyl-1H-pyrazole-4-carboxamide, N-cyclopropyl-3-(difluoromethyl)-N-(2-ethyl-4,5-dimethylbenzyl)-5-fluoro-1-methyl-1H-pyrazole-4-carboxamide, N-cyclopropyl-3-(difluoromethyl)-N-(2-ethyl-5-fluorobenzyl)-5-fluoro-1-methyl-1H-pyrazole-4-carboxamide, N-cyclopropyl-3-(difluoromethyl)-N-(2-ethyl-5-methylbenzyl)-5-fluoro-1-methyl-1H-pyrazole-4-carboxamide, N-cyclopropyl-N-(2-cyclopropyl-5-fluorobenzyl)-3-(difluoromethyl)-5-fluoro-1-methyl-1H-pyrazole-4-carboxamide, N-cyclopropyl-N-(2-cyclopropyl-5-methylbenzyl)-3-(difluoromethyl)-5-fluoro-1-methyl-1H-pyrazole-4-carboxamide, N-cyclopropyl-N-(2-cyclopropylbenzyl)-3-(difluoromethyl)-5-fluoro-1-methyl-1H-pyrazole-4-carboxamide, pyrapropoyne, N-[rac-(1S,2S)-2-(2,4-dichlorophenyl)cyclobutyl]-2-(trifluoromethyl)nicotinamide, and N-[(1S,2S)-2-(2,4-dichlorophenyl)cyclobutyl]-2-(trifluoromethyl)nicotinamide; (3) Inhibitors of the respiratory chain at complex III, e.g., ametoctradin, amisulbrom, azoxystrobin, coumethoxystrobin, coumoxystrobin, cyazofamid, dimoxystrobin, enoxastrobin, famoxadone, fenamidone, flufenoxystrobin, fluoxastrobin, kresoxim-methyl, metominostrobin, orysastrobin, picoxystrobin, pyraclostrobin, pyrametostrobin, pyraoxystrobin, trifloxystrobin, (2E)-2-{2-[({[(IE)-1-(3-{[(E)-1-fluoro-2-phenylvinyl]oxy}phenyl)ethylidene]amino}oxy)methyl]phenyl}-2-(methoxyimino)-N-methylacetamide, (2E,3Z)-5-{[1-(4-chlorophenyl)-1H-pyrazol-3-yl]oxy}-2-(methoxyimino)-N,3-dimethylpent-3-enamide, (2R)-2-{2-[(2,5-dimethylphenoxy)methyl]phenyl}-2-methoxy-N-methylacetamide, (2S)-2-{2-[(2,5-dimethylphenoxy)methyl]phenyl}-2-methoxy-N-methylacetamide, fenpicoxamid, mandestrobin, N-(3-ethyl-3,5,5-trimethylcyclohexyl)-3-formamido-2-hydroxybenzamide, (2E,3Z)-5-{[1-(4-chloro-2-fluorophenyl)-1H-pyrazol-3-yl]oxy}-2-(methoxyimino)-N,3-dimethylpent-3-enamide, methyl {5-[3-(2,4-dimethylphenyl)-1H-pyrazoll-yl]-2-methylbenzyl}carbamate, metyltetraprole, and florylpicoxamid; (4) Inhibitors of the mitosis and cell division, e.g., carbendazim, diethofencarb, ethaboxam, fluopicolide, pencycuron, thiabendazole, thiophanate methyl, zoxamide, pyridachlometyl, 3-chloro-5-(4-chlorophenyl)-4-(2,6-difluorophenyl)-6-methylpyridazine, 3-chloro-5-(6-chloropyridin-3-yl)-6-methyl-4-(2,4,6-trifluorophenyl)pyridazine, 4-(2-bromo-4-fluorophenyl)-N-(2,6-difuorophenyl)-1,3-dimethyl 1H-pyrazol-5-amine, 4-(2-bromo-4-fluorophenyl)-N-(2-bromo-6-fluorophenyl)-1,3-dimethyl-1H-pyrazol-5-amine, 4-(2-bromo-4-fluorophenyl)-N-(2-bromophenyl)-1,3-dimethyl-1H-pyrazol-5-amine, 4-(2-bromo-4-fluorophenyl)-N-(2-chloro-6-fluorophenyl)-1,3-dimethyl-1H-pyrazol-5-amine, 4-(2-bromo-4-fluorophenyl)-N-(2-chlorophenyl)-1,3-dimethyl-1H-pyrazol-5-amine, 4-(2-bromo-4-fluorophenyl)-N-(2-fluorophenyl)-1,3-dimethyl-1H-pyrazol-5-amine, 4-(2-chloro-4-fluorophenyl)-N-(2,6-difluorophenyl)-1,3-dimethyl-1H-pyrazol-5-amine, 4-(2-chloro-4-fluorophenyl)-N-(2-chloro-6-fluorophenyl)-1,3-dimethyl-1H-pyrazol-5-amine, 4-(2-chloro-4-fluorophenyl)-N-(2-chlorophenyl)-1,3-dimethyl-1H-pyrazol-5-amine, 4-(2-chloro-4-fluorophenyl)-N-(2-fluorophenyl)-1,3-dimethyl-1H-pyrazol-5-amine, 4-(4-chlorophenyl)-5-(2,6-difluorophenyl)-3,6-dimethylpyridazine, N-(2-bromo-6-fluorophenyl)-4-(2-chloro-4-fluorophenyl)-1,3-dimethyl-1H-pyrazol-5-amine, N-(2-bromophenyl)-4-(2-chloro-4-fluorophenyl)-1,3-dimethyl-1H-pyrazol-5-amine, N-(4-chloro-2,6-difluorophenyl)-4-(2-chloro-4-fluorophenyl)-1,3-dimethyl-1H-pyrazol-5-amine, and fluopimomide; (5) Fungicide with multisite action, e.g., bordeaux mixture, captafol, captan, chlorothalonil, copper hydroxide, copper naphthenate, copper oxide, copper oxychloride, copper(2+) sulfate, dithianon, dodine, folpet, mancozeb, maneb, metiram, metiram zinc, oxinecopper, propineb, sulfur and sulfur preparations including calcium polysulfide, thiram, zineb, ziram, and 6-ethyl-5,7-dioxo-6,7-dihydro-5H-pyrrolo[3′,4′:5,6][1,4]dithiino[2,3-c][1,2]thiazole-3-carbonitrile; (6) Fungicides inducing host defense, e.g., acibenzolar-S-methyl, isotianil, probenazole, and tiadinil; (7) Inhibitors of amino acid and/or protein biosynthesis, e.g., cyprodinil, kasugamycin, kasugamycin hydrochloride hydrate, oxytetracycline, and pyrimethanil, 3-(5-fluoro-3,3,4,4-tetramethyl-3,4-dihydroisoquinolin-1-yl)quinoline; (8) Inhibitors of ATP production, e.g., silthiofam; (9) Inhibitors of cell wall synthesis, e.g., benthiavalicarb, dimethomorph, flumorph, iprovalicarb, mandipropamid, pyrimorph, valifenalate, (2E)-3-(4-tert-butylphenyl)-3-(2-chloropyridin-4-yl)-1-(morpholin-4-yl)prop-2-en-1-one, and (2Z)-3-(4-tert-butylphenyl)-3-(2-chloropyridin-4-yl)-1-(morpholin-4-yl)prop-2-en-1-one; (10) Inhibitors of lipid and/or membrane synthesis, e.g., propamocarb, propamocarb hydrochloride, and tolclofos-methyl; (11) Inhibitors of melanin biosynthesis, e.g., tricyclazole and tolprocarb; (12) Inhibitors of nucleic acid synthesis, e.g., benalaxyl, benalaxyl-M (kiralaxyl), metalaxyl, and metalaxyl-M (mefenoxam); (13) Inhibitors of signal transduction, e.g., fludioxonil, iprodione, procymidone, proquinazid, quinoxyfen, and vinclozolin; (14) Uncouplers, e.g., fluazinam and meptyldinocap; and (15) Miscellaneous fungicidal or antifungal compounds, e.g., abscisic acid, benthiazole, bethoxazin, capsimycin, carvone, chinomethionat, cufraneb, cyflufenamid, cymoxanil, cyprosulfamide, flutianil, fosetylaluminium, fosetyl-calcium, fosetyl-sodium, methyl isothiocyanate, metrafenone, mildiomycin, natamycin, nickel dimethyldithiocarbamate, nitrothal-isopropyl, oxamocarb, oxathiapiprolin, oxyfenthiin, pentachlorophenol and salts, phosphorous acid and its salts, propamocarbfosetylate, pyriofenone (chlazafenone), tebufloquin, tecloftalam, tolnifanide, 1-(4-[4-[(5R)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, 1-(4-[4-[(5S)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, 2-(6-benzylpyridin-2-yl)quinazoline, dipymetitrone, 2-[3,5-bis(difluoromethyl)-1H-pyrazol-1-yl]-1-[4-(4-{5-[2-(prop-2-yn-1-yloxy)phenyl]-4,5-dihydro-1,2-oxazol-3-yl}-1,3-thiazol-2-yl)piperidin-1-yl]ethanone, 2-(3,5-bis(difluoromethyl)-1H-pyrazol-1-yl]-1-[4-(4-{5-[2-chloro-6-(prop-2-yn-1-yloxy)phenyl]-4,5-dihydro-1,2-oxazol-3-yl}-1,3-thiazol-2-yl)piperidin-1-yl]ethanone, 2-[3,5-bis(difluoromethyl)-1H-pyrazol-1-yl]-1-[4-(4-[5-[2-fluoro-6-(prop-2-yn-1-yloxy)phenyl]-4,5-dihydro-1,2-oxazol-3-yl}-1,3-thiazol-2-yl)piperidin-1-yl]ethanone, 2-[6-(3-fluoro-4-methoxyphenyl)-5-methylpyridin-2-yl]quinazoline, 2-{(5R)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenylmethanesulfonate, 2-{(5S)-3-[2-(1-([3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulfonate, ipflufenoquin, 2-{2-fluoro-6-[(8-fluoro-2-methylquinolin-3-yl)oxy]phenyl}propan-2-ol, fluoxapiprolin, 2-{3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl[phenyl methanesulfonate, 2-phenylphenol and salts, 3-(4,4,5-trifluoro-3,3-dimethyl-3,4-dihydroisoquinolin-1-yl)quinoline, quinofumelin, 4-amino-5-fluoropyrimidin-2-ol (tautomeric form: 4-amino-5-fluoropyrimidin-2(1H)-one), 4-oxo-4-[(2-phenylethyl)amino]butanoic acid, 5-amino-1,3,4-thiadiazole-2-thiol, 5-chloro-N′-phenyl-N′-(prop-2-yn-1-yl)thiophene-2-sulfonohydrazide, 5-fluoro-2-[(4-fluorobenzyl)oxy]-pyrimidin-4-amine, 5-fluoro-2-[(4-methylbenzyl)oxy]pyrimidin-4-amine, 9-fluoro-2,2-dimethyl-5-(quinolin-3-yl)-2,3-dihydro-1,4-benzoxazepine, but-3-yn-1-yl {6-[({[(Z)-(1-methyl-1H-tetrazol-5-yl)(phenyl)methylene]amino}oxy)methyl]pyridin-2-yl}carbamate, ethyl (2Z)-3-amino-2-cyano-3-phenylacrylate, phenazine-1-carboxylic acid, propyl 3,4,5-trihydroxybenzoate, quinolin-8-ol, quinolin-8-ol sulfate (2:1), tert-butyl {6-[({[(1-methyl-1H-tetrazol-5-yl)(phenyl)methylene]amino}oxy)methyl]pyridin-2-yl}carbamate, 5-fluoro-4-imino-3-methyl-1-[(4-methylphenyl)sulfonyl]-3,4-dihydropyrimidin-2 (1H)-one, aminopyrifen, (N′-[2-chloro-4-(2-fluorophenoxy)-5-methylphenyl]-N-ethyl-N-methylimidoformamide), (N′-(2-chloro-5-methyl-4-phenoxyphenyl)-N-ethyl-N-methylimidoformamide), (2-{2-[(7,8-difluoro-2-methylquinobn-3-yl)oxy]-6-fluorophenyl}propan-2-ol), (5-bromo-1-(5,6-dimethylpyridin-3-yl)-3,3-dimethyl-3,4-dihydroisoquinobne), (3-(4,4-difluoro-5,5-dimethyl-4,5-dihydrothieno[2,3-c]pyridin-7-yl)quinoline), (1-(4,5-dimethyl-1H-benzimidazol-1-yl)-4,4-difluoro-3,3-dimethyl-3,4-dihydroisoquinoline), 8-fluoro-3-(5-fluoro-3,3-dimethyl-3,4-dihydroisoqmnolin-1-yl)quinolone, 8-fluoro-3-(5-fluoro-3,3,4,4-tetramethyl-3,4-dihydroisoquinolin-1-yl)quinolone, 3-(4,4-difluoro-3,3-dimethyl-3,4-dihydroisoquinolin-1-yl)-8-fluoroquinoline, (N-methyl-N-phenyl-4-[5-(trifluoromethyl)-1,2,4-oxadiazol-3-yl]benzamide), methyl {4-[5-(trifluoromethyl)-1,2,4-oxadiazol-3-yl]phenyl}carbamate, (N-{4-[5-(trifluoromethyl)-1,2,4-oxadiazol-3-yl]benzyl}cyclopropanecarboxamide), N-methyl-4-(5-(trifluoromethyl)-1,2,4-oxadiazol-3-yl]benzamide, N-[(E)-methoxyiminomethyl]-4-[5-(trifluoromethyl)-1,2,4-oxadiazol-3-yl]benzamide, N-[(Z)-methoxyiminomethyl]-4-[5-(trifluoromethyl)-1,2,4-oxadiazol-3-yl]benzamide, N-[4-[5-(trifluoromethyl)-1,2,4-oxadiazol-3-yl]phenyl]cyclopropanecarboxamide, N-(2-fhiorophenyl)-4-[5-(trifluoromethyl)-1,2,4-oxadiazol-3-yl]benzamide, 2,2-difluoro-N-methyl-2-[4-[5-(trifluoromethyl)-1,2,4-oxadiazol-3-yl]phenyl]acetamide, N-allyl-N-[[4-[5-(trifluoromethyl)-1,2,4-oxadiazol-3-yl)phenyl]methyl]acetamide, N-[(E)-N-methoxy-C-methyl-carbonimidoyl]-4-(5-(trifluoromethyl)-1,2,4-oxadiazol-3-yl]benzamide, N-[(Z)—N-methoxy-C-methyl-carbonimidoyl]-4-[5-(trifluoromethyl)-1,2,4-oxadiazol-3-yl]benzamide, N-allyl-N-[[4-[5-(trifluoromethyl)-1,2,4-oxadiazol-3-yl]phenyl]methyl]propanamide, 4,4-dimethyl-1-[[4-[5-(trifluoromethyl)-1,2,4-oxadiazol-3-yl]phenyl]methyl]pyrrolidin-2-one, N-methyl-4-[5-(trifluoromethyl)-1,2,4-oxadiazol-3-yl]benzenecarbothioamide, 5-methyl-1-[[4-[5-(trifluoromethyl)-1,2,4-oxadiazol-3-yl]phenyl]methyl]pyrrolidin-2-one, N-((2,3-difluoro-4-[5-(trifluoromethyl)-1,2,4-oxadiazol-3-yl]phenyl]methyl]-3,3,3-trifluoro-propanamide, 1-methoxy-1-methyl-3-[[4-[5-(trifluoromethyl}-1,2,4-oxadiazol-3-yl]phenyl]methyl]urea, 1,1-diethyl-3-[[4-[5-(trifluoromethyl}-1,2,4-oxadiazol-3-yl]phenyl]methyl]urea, N-[[4-[5-(trifluoromethyl)-1,2,4-oxadiazol-3-yl]phenyl]methyl]propanamide, N-methoxy-N-[[4-[5-(trifluoromethyl)-1,2,4-oxadiazol-3-yl]phenyl]-methyl]cyclopropanecarboxamide, 1-methoxy-3-methyl-1-[[4-[5-(trifluoromethyl)-1,2,4-oxadiazol-3-yl]phenyl]methyl]urea, N-methoxy-N-[[4-[5-(trifluoromethyl)-1,2,4-oxadiazol-3-yl]phenyl]methyl)cyclopropanecarboxamide, N,2-dimethoxy-N-[[4-[5-(trifluoromethyl)-1,2,4-oxadiazol-3-yl]phenyl]methyl]propanamide, N-ethyl-2-methyl-N-[[4-[5-(trifluoromethyl)-1,2,4-oxadiazol-3-yl)phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, 3-ethyl-1-methoxy-1-[[4-[5-(trifluoromethyl)-1,2,4-oxadiazol-3-yl]phenyl]methyl]urea, 1-[[4-[5-(trifluoromethyl)-1,2,4-oxadiazol-3-yl]phenyl]-methyl]piperidin-2-one, 4,4-dimethyl-2-[[4-[5-(trifluoromethyl)-1,2,4-oxadiazol-3-yl]phenyl]-methyl]isooxazolidin-3-one, 5,5-dimethyl-2-[[4-[5-(trifluoromethyl)-1,2,4-oxadiazol-3-yl]phenyl]methyl]isoxazolidin-3-one, 3,3-dimethyl-1-[[4-[5-(trifluoromethyl)-1,2,4-oxadiazol-3-yl]phenyl]methyl]piperidin-2-one, 1-[[3-fluoro-4-(5-(trifluoromethyl)-1,2,4-oxadiazol-3-yl]-phenyl]methyl]azepan-2-one, 4,4-dimethyl-2-[[4-(5-(trifluoromethyl)-1,2,4-oxadiazol-3-yl]-phenyl]methyl]isoxazolidin-3-one, 5,5-dimethyl-2-[[4-[5-(trifluoromethyl)-1,2,4-oxadiazol-3-yl]phenyl]methyl]isoxazolidin-3-one, ethyl 1-{4-[5-(trifluoromethyl)-1,2,4-oxadiazol-3-yl]benzyl}-1H-pyrazole-4-carboxylate, N,N-dimethyl-1-{4-[5-(trifluoromethyl)-1,2,4-oxadiazol-3-yl]benzyl}-1H-1,2,4-triazol-3-amine, N-{2,3-difluoro-4-[5-(trifluoromethyl)-1,2,4-oxadiazol-3-yl]benzyl}butanamide, N-(1-methylcyclopropyl)-4-[5-(trifluoromethyl)-1,2,4-oxadiazol-3-yl]benzamide, N-(2,4-difluorophenyl)-4-[5-(trifluoromethyl)-1,2,4-oxadiazol-3-yl]benzamide, 1-(5,6-dimethylpyridin-3-yl)-4,4-difluoro-3,3-dimethyl-3,4-dihydroisoquinoline, 1-(6-(difluoromethyl)-5-methyl-pyridin-3-yl)-4,4-difluoro-3,3-dimethyl-3,4-dihydroisoquinoline, 1-(5-(fluoromethyl)-6-methyl-pyridin-3-yl)-4,4-difluoro-3,3-dimethyl-3,4-dihydroisoquinoline, 1-(6-(difluoromethyl)-5-methoxy-pyridin-3-yl)-4,4-difluoro-3,3-dimethyl-3,4-dihydroisoquinoline, 4-[5-(trifluoromethyl)-1,2,4-oxadiazol-3-yl]phenyl dimethylcarbamate, N-{4-[5-(trifhioromethyl)-1,2,4-oxadiazol-3-yl]phenyl}propanamide, 3-[2-(1-{[5-methyl-3-(trifluoromethyl)-1H-pyrazol-1-yl]acetyl}piperidin-4-yl)-1,3-thiazol-4-yl]-1,5-dihydro-2,4-benzodioxepin-6-yl methanesulfonate, 9-fluoro-3-[2-(1-{[5-methyl-3-(trifluoromethyl)-1H-pyrazol-1-yl acetyl}piperidin-4-yl)-1,3-thiazol-4-yl]-1,5-dihydro-2,4-benzodioxepin-6-ylmethanesulfonate, 3-[2-(1-{[3,5-bis (difluoromethyl)-1H-pyrazol-1-yl]acetyl}piperidin-4-yl)-1,3-thiazol-4-yl]-1,5-dihydro-2,4-benzodioxepin-6-yl methanesulfonate, 3-[2-(1-{[3,5-bis(difluoromethyl)-1H-pyrazol-1-yl]acetyl}piperidin-4-yl)-1,3-thiazol-4-yl]-9-fluoro-1,5-dihydro-2,4-benzodioxepin-6-yl methanesulfonate, 1-(6,7-dimethylpyrazolo[1,5-a]pyridin-3-yl)-4,4-difluoro-3,3-dimethyl-3,4-dihydroisoquinohne, 8-fluoro-N-(4,4,4-trifluoro-2-methyl-1-phenylbutan-2-yl)quinoline-3-carboxamide, 8-fluoro-N-[(2S)-4,4,4-trifluoro-2-methyl-1-phenylbutan-2-yl]quinoline-3-carboxamide, N-(2,4-dimethyl-1-phenylpentan-2-yl)-8-fluoroquinoline-3-carboxamide and N-[(2S)-2,4-dimethyl-1-phenylpentan-2-yl]-8-fluoroquinoline-3-carboxamide, flufenoxadiazam, flumetylsulforim, fluoxytioconazole, metarylpicoxamid, rimisoxafen, chloroinconazide, cyclobutrifluram, and flubeneteram.

Although the foregoing disclosure describes aspects and embodiments of the invention in some detail by way of illustration and examples, the description and examples should not be construed as limiting the scope of the invention. Further embodiments are disclosed within the claims. The disclosures of all patent and scientific literature cited in this disclosure are expressly incorporated by reference in their entirety herein.

1. A method of decreasing growth or reproduction of a fungus, comprising:
providing a fungus with an antifungal composition that comprises:
(a) an effective amount of at least one conidial germination-inhibiting (CGI) factor, CGI factor precursor, CGI factor fragment, or CGI factor motif, wherein the CGI factor, CGI factor precursor, CGI factor fragment, or CGI factor motif comprises:
(i) an alpha pheromone fused at its C terminus to a polyarginine cell-penetrating peptide, optionally having a sequence selected from the group consisting of WHWLQLKPGQPMYRRRRRRRR (SEQ ID NO:2240 or SEQ ID NO:5752), WHWLQLKPGQPMYRRRRRRRRR (SEQ ID NO:5758), SEQ ID NO:5750, 5751, 5754, 5755, 5756, 5757, 5759, 5760, and 5761; or
(ii) a fusion peptide comprising an alpha pheromone that is fused at its C terminus to a cell-penetrating peptide, wherein the alpha pheromone has a sequence selected from the group consisting of SEQ ID NOs:2182-2186, and wherein the cell-penetrating peptide has a sequence selected from the group consisting of SEQ ID NOs:5736-5749;
(iii) a homodimer of an alpha pheromone, with or without a neutral amino-acid linker, optionally wherein the homodimer is a peptide with a sequence selected from the group consisting of WHWLQLKPGQPMYWHWLQLKPGQPMY (SEQ ID NO:2188), WHWLQLKPGQPMYGGGGWHWLQLKPGQPMY (SEQ ID NO:5837), SEQ ID NOs: 5835-5840, 2189, 5841-5845, and 5846-5911; or
(iv) an amino acid sequence that has at least 80% sequence identity with a sequence selected from the group consisting of SEQ ID NO: 961-1920, SEQ ID NO: 1921-2189, SEQ ID NO: 2194-2210, SEQ ID NO: 2215-2243, SEQ ID NO: 2457-3361, SEQ ID NO: 5707-5731, and SEQ ID NO: 5755-5911; and
wherein the nucleotide sequence encoding the at least one CGI factor, CGI factor precursor, CGI factor fragment, or CGI factor motif does not occur in the genome of the fungus;
(b) an effective amount of at least one fungicide selected from the group consisting of:
(i) a fungicide selected from the group consisting of drosomycin, metconazole, and natamycin;
(ii) a fungicide that interferes with fungal cell wall or fungal cell membranes;
(iii) a fungal signal transduction-inhibiting fungicide;
(iv) a fungal respiration-inhibiting fungicide; and
(v) a fungal amino acid and/or protein synthesis-inhibiting fungicide; and
(c) optionally, an agriculturally or pharmaceutically acceptable carrier;
whereby the growth or reproduction of the fungus is decreased, relative to a control fungus not provided with the antifungal composition.

2. The method of claim 1, wherein the decreased growth or reproduction of the fungus comprises reduced spore germination and/or reduced hyphal growth.

3. A method of preventing or reducing disease caused by a fungus that is a fungal pathogen of a plant, comprising:
contacting one or more cells of the plant with an antifungal composition that comprises
(a) an effective amount of at least one conidial germination-inhibiting (CGI) factor, CGI factor precursor, CGI factor fragment, or CGI factor motif, wherein the CGI factor, CGI factor precursor, CGI factor fragment, or CGI factor motif comprises:
(i) an alpha pheromone fused at its C terminus to a polyarginine cell-penetrating peptide, optionally having a sequence selected from the group consisting of WHWLQLKPGQPMYRRRRRRRR (SEQ ID NO:2240 or SEQ ID NO:5752), WHWLQLKPGQPMYRRRRRRRRR (SEQ ID NO:5758), SEQ ID NO:5750, 5751, 5754, 5755, 5756, 5757, 5759, 5760, and 5761; or
(ii) a fusion peptide comprising an alpha pheromone that is fused at its C terminus to a cell-penetrating peptide, wherein the alpha pheromone has a sequence selected from the group consisting of SEQ ID NOs:2182-2186, and wherein the cell-penetrating peptide has a sequence selected from the group consisting of SEQ ID NOs:5736-5749; or
(iii) an amino acid sequence that has at least 80% sequence identity with a sequence selected from the group consisting of SEQ ID NO: 961-1920, SEQ ID NO: 1921-2189, SEQ ID NO: 2194-2210, SEQ ID NO: 2215-2243, SEQ ID NO: 2457-3361, SEQ ID NO: 5707-5731, and SEQ ID NO: 5755-5911; and
wherein the nucleotide sequence encoding the at least one CGI factor, CGI factor precursor, CGI factor fragment, or CGI factor motif does not occur in the genome of the fungal pathogen;
(b) an effective amount of at least one fungicide selected from the group consisting of:
(i) a fungicide selected from the group consisting of drosomycin, metconazole, and natamycin;
(ii) a fungicide that interferes with fungal cell wall or fungal cell membranes;
(iii) a fungal signal transduction-inhibiting fungicide;
(iv) a fungal respiration-inhibiting fungicide; and
(v) a fungal amino acid and/or protein synthesis-inhibiting fungicide; and
(c) optionally, an agriculturally or pharmaceutically acceptable carrier;
whereby disease caused by the fungal pathogen is prevented or decreased in the plant, relative to a control plant not contacted with the antifungal composition.

4. A method of treating a subject with a disease caused by a fungus, comprising:
administering to the subject an antifungal composition that comprises
(a) an effective amount of at least one conidial germination-inhibiting (CGI) factor, CGI factor precursor, CGI factor fragment, or CGI factor motif, wherein the CGI factor, CGI factor precursor, CGI factor fragment, or CGI factor motif comprises:
(i) an alpha pheromone fused at its C terminus to a polyarginine cell-penetrating peptide, optionally having a sequence selected from the group consisting of WHWLQLKPGQPMYRRRRRRRR (SEQ ID NO:2240 or SEQ ID NO:5752), WHWLQLKPGQPMYRRRRRRRRR (SEQ ID NO:5758), SEQ ID NO:5750, 5751, 5754, 5755, 5756, 5757, 5759, 5760, and 5761; or
(ii) a fusion peptide comprising an alpha pheromone that is fused at its C terminus to a cell-penetrating peptide, wherein the alpha pheromone has a sequence selected from the group consisting of SEQ ID NOs:2182-2186, and wherein the cell-penetrating peptide has a sequence selected from the group consisting of SEQ ID NOs:5736-5749; or
(iii) an amino acid sequence that has at least 80% sequence identity with a sequence selected from the group consisting of SEQ ID NO: 961-1920, SEQ ID NO: 1921-2189, SEQ ID NO: 2194-2210, SEQ ID NO: 2215-2243, SEQ ID NO: 2457-3361, SEQ ID NO: 5707-5731, and SEQ ID NO: 5755-5911; and
wherein the nucleotide sequence encoding the at least one CGI factor, CGI factor precursor, CGI factor fragment, or CGI factor motif does not occur in the genome of the fungus;
(b) an effective amount of at least one fungicide selected from the group consisting of:
(i) a fungicide selected from the group consisting of drosomycin, metconazole, and natamycin;
(ii) a fungicide that interferes with fungal cell wall or fungal cell membranes;
(iii) a fungal signal transduction-inhibiting fungicide;
(iv) a fungal respiration-inhibiting fungicide; and
(v) a fungal amino acid and/or protein synthesis-inhibiting fungicide; and
(c) optionally, an agriculturally or pharmaceutically acceptable carrier;
whereby the disease is prevented or decreased in the subject, relative to a control subject not provided with the antifungal composition.

5. The method of claim 4, wherein the subject is an animal, optionally wherein the animal is a non-human mammal.

6. The method of any of claims 1, 3, or 4, wherein the antifungal composition comprises:
(a) a CGI factor having the sequence of WHWLQLKPGQPMYRRRRRRRR (SEQ ID NO:2240 or SEQ ID NO:5752) or WHWLQLKPGQPMYRRRRRRRRR (SEQ ID NO:5758);
(b) an effective amount of a fungicide selected from the group consisting of drosomycin, metconazole, and natamycin; and
(c) optionally, an agriculturally or pharmaceutically acceptable carrier.

7. The method of any of claims 1, 3, or 4, wherein the fungicide's minimum inhibitory concentration (MIC) is reduced in the presence of the CGI factor relative to the fungicide's MIC in the absence of the CGI factor.

8. The method of any of claims 1, 3, or 4, wherein the fungus is a Fusarium sp. or a Botrytis sp.

9. An antifungal composition comprising:
(a) an effective amount of at least one conidial germination-inhibiting (CGI) factor, CGI factor precursor, CGI factor fragment, or CGI factor motif, wherein the CGI factor, CGI factor precursor, CGI factor fragment, or CGI factor motif comprises:
(i) an alpha pheromone fused at its C terminus to a polyarginine cell-penetrating peptide, optionally having a sequence selected from the group consisting of WHWLQLKPGQPMYRRRRRRRR (SEQ ID NO:2240 or SEQ ID NO:5752), WHWLQLKPGQPMYRRRRRRRRR (SEQ ID NO:5758), SEQ ID NO:5750, 5751, 5754, 5755, 5756, 5757, 5758, 5759, 5760, and 5761; or
(ii) a fusion peptide comprising an alpha pheromone that is fused at its C terminus to a cell-penetrating peptide, wherein the alpha pheromone has a sequence selected from the group consisting of SEQ ID NOs:2182-2186, and wherein the cell-penetrating peptide has a sequence selected from the group consisting of SEQ ID NOs:5736-5749; or
(iii) an amino acid sequence that has at least 80% sequence identity with a sequence selected from the group consisting of SEQ ID NO: 961-1920, SEQ ID NO: 1921-2189, SEQ ID NO: 2194-2210, SEQ ID NO: 2215-2243, SEQ ID NO: 2457-3361, SEQ ID NO: 5707-5731, and SEQ ID NO: 5755-5911; and
wherein the nucleotide sequence encoding the at least one CGI factor, CGI factor precursor, CGI factor fragment, or CGI factor motif does not occur in the genome of the fungus;
(b) an effective amount of at least one fungicide selected from the group consisting of:
(i) a fungicide selected from the group consisting of drosomycin, metconazole, and natamycin;
(ii) a fungicide that interferes with fungal cell wall or fungal cell membranes;
(iii) a fungal signal transduction-inhibiting fungicide;
(iv) a fungal respiration-inhibiting fungicide; and
(v) a fungal amino acid and/or protein synthesis-inhibiting fungicide; and
(c) optionally, an agriculturally or pharmaceutically acceptable carrier.

10. The antifungal composition of claim 9, comprising:
(a) an effective amount of a fusion peptide comprising an alpha pheromone that is fused at its C terminus to a cell-penetrating peptide, wherein the alpha pheromone has a sequence selected from the group consisting of SEQ ID NOs:2182-2186, and wherein the cell-penetrating peptide is a polyarginine cell-penetrating peptide or has a sequence selected from the group consisting of SEQ ID NOs:5736-5749;
(b) an effective amount of a fungicide selected from the group consisting of drosomycin, metconazole, and natamycin; and
(c) optionally, an agriculturally or pharmaceutically acceptable carrier.

11. The antifungal composition of claim 9, comprising:
(a) an effective amount of a CGI factor having the sequence of WHWLQLKPGQPMYRRRRRRRR (SEQ ID NO:2240 or SEQ ID NO:5752) or WHWLQLKPGQPMYRRRRRRRRR (SEQ ID NO:5758);
(b) an effective amount of a fungicide selected from the group consisting of drosomycin, metconazole, and natamycin; and
(c) optionally, an agriculturally or pharmaceutically acceptable carrier.

12. The antifungal composition of claim 9, formulated for agricultural use as a seed treatment, a foliar spray treatment, a foliar drench treatment, an injectable formulation, a Ready-To-Use (RTU) formulation, a produce coating, a suspension concentrate, a tank-mix, an aerosol, a root dip, a drench, a fog, a soil treatment, an irrigation formulation, or a sprinkler formulation, or formulated for pharmaceutical use as a liquid, a gel, an emulsion, a suspension, an encapsulation, a solid, a powder, a coating, a spray, or an injectable.
